SEQUENCE LISTING (4 sequences)

SEQ ID NO: 1 - DNA, 4426 bp, Homo sapiens
  CDS (67)..(2439)
tgccttgaccaggacttgggactttgcgaaaggatcgcggggcccggagaggtgttggagagcacaatggctgaacaagtccttcctcaggctttgtatttgagcaatatgcggaaagctgtgaagatacgggagagaactccagaagacatttttaaacctactaatgggatcattcatcattttaaaaccatgcaccgatacacactggaaatgttcagaacttgccagttttgtcctcagtttcgggagatcatccacaaagccctcatcgacagaaacatccaggccaccctggaaagccagaagaaactcaactggtgtcgagaagtccggaagcttgtggcgctgaaaacgaacggtgacggcaattgcctcatgcatgccacttctcagtacatgtggggcgttcaggacacagacttggtactgaggaaggcgctgttcagcacgctcaaggaaacagacacacgcaactttaaattccgctggcaactggagtctctcaaatctcaggaatttgttgaaacggggctttgctatgatactcggaactggaatgatgaatgggacaatcttatcaaaatggcttccacagacacacccatggcccgaagtggacttcagtacaactcactggaagaaatacacatatttgtcctttgcaacatcctcagaaggccaatcattgtcatttcagacaaaatgctaagaagtttggaatcaggttccaatttcgcccctttgaaagtgggtggaatttacttgcctctccactggcctgcccaggaatgctacagataccccattgttctcggctatgacagccatcattttgtacccttggtgaccctgaaggacagtgggcctgaaatccgagctgttccacttgttaacagagaccggggaagatttgaagacttaaaagttcactttttgacagatcctgaaaatgagatgaaggagaagctcttaaaagagtacttaatggtgatagaaatccccgtccaaggctgggaccatggcacaactcatctcatcaatgccgcaaagttggatgaagctaacttaccaaaagaaatcaatctggtagatgattactttgaacttgttcagcatgagtacaagaaatggcaggaaaacagcgagcaggggaggagagaggggcacgcccagaatcccatggaaccttccgtgccccagctttctctcatggatgtaaaatgtgaaacgcccaactgccccttcttcatgtctgtgaacacccagcctttatgccatgagtgctcagagaggcggcaaaagaatcaaaacaaactcccaaagctgaactccaagccgggccctgaggggctccctggcatggcgctcggggcctctcggggagaagcctatgagcccttggcgtggaaccctgaggagtccactggggggcctcattcggccccaccgacagcacccagcccttttctgttcagtgagaccactgccatgaagtgcaggagccccggctgccccttcacactgaatgtgcagcacaacggattttgtgaacgttgccacaacgcccggcaacttcacgccagccacgccccagaccacacaaggcacttggatcccgggaagtgccaagcctgcctccaggatgttaccaggacatttaatgggatctgcagtacttgcttcaaaaggactacagcagaggcctcctccagcctcagcaccagcctccctccttcctgtcaccagcgttccaagtcagatccctcgcggctcgtccggagcccctccccgcattcttgccacagagctggaaacgacgcccctgctggctgcctgtctcaagctgcacggactcctggggacaggacggggacgagcaagtgcagaaaagccggctgcgtgtattttgggactccagaaaacaagggcttttgcacactgtgtttcatcgagtacagagaaaacaaacattttgctgctgcctcagggaaagtcagtcccacagcgtccaggttccagaacaccattccgtgcctggggagggaatgcggcacccttggaagcaccatgtttgaaggatactgccagaagtgtttcattgaagctcagaatcagagatttcatgaggccaaaaggacagaagagcaactgagatcgagccagcgcagagatgtgcctcgaaccacacaaagcacctcaaggcccaagtgcgcccgggcctcctgcaagaacatcctggcctgccgcagcgaggagctctgcatggagtgtcagcatcccaaccagaggatgggccctggggcccaccggggtgagcctgcccccgaagacccccccaagcagcgttgccgggcccccgcctgtgatcattttggcaatgccaagtgcaacggctactgcaacgaatgctttcagttcaagcagatgtatggctaaccggaaacaggtgggtcacctcctgcaagaagtggggcctcgagctgtcagtcatcatggtgctatcctctgaacccctcagctgccactgcaacagtgggcttaagggtgtctgagcaggagaggaaagataagctcttcgtggtgcccacgatgctcaggtttggtaacccgggagtgttcccaggtggccttagaaagcaaagcttgtaactggcaagggatgatgtcagattcagcccaaggttcctcctctcctaccaagcaggaggccaggaacttctttggacttggaaggtgtgcggggactggccgaggcccctgcaccctgcgcatcaggactgcttcatcgtcttggctgagaaagggaaaagacacacaagtcgcgtgggttggagaagccagagccattccacctcccctcccccagcatctctcagagatgtgaagccagatcctcatggcagcgaggccctctgcaagaagctcaaggaagctcagggaaaatggacgtattcagagagtgtttgtagttcatggtttttccctacctgcccggttcctttcctgaggacccggcagaaatgcagaaccatccatggactgtgattctgaggctgctgagactgaacatgttcacattgacagaaaaacaagctgctctttataatatgcaccttttaaaaaattagaatattttactgggaagacgtgtaactctttgggttattactgtctttacttctaaagaagttagcttgaactgaggagtaaaagtgtgtacatatataatatacccttacattatgtatgagggatttttttaaattatattgaaatgctgccctagaagtacaataggaaggctaaataataataacctgttttctggttgttgttggggcatgagcttgtgtatacactgcttgcataaactcaaccagctgcctttttaaagggagctctagtcctttttgtgtaattcactttatttattttattacaaacttcaagattatttaagtgaagatatttcttcagctctggggaaaatgccacagtgttctcctgagagaacatccttgctttgagtcaggctgtgggcaagttcctgaccacagggagtaaattggcctctttgatacacttttgcttgcctccccaggaaagaaggaattgcatccaaggtatacatacatattcatcgatgtttcgtgcttctccttatgaaactccagctatgtaataaaaaactatactctgtgttctgttaatgcctctgagtgtcctacctccttggagatgagatagggaaggagcagggatgagactggcaatggtcacagggaaagatgtggccttttgtgatggttttattttctgttaacactgtgtcctgggggggctgggaagtcccctgcatcccatggtaccctggtattgggacagcaaaagccagtaaccatgagtatgaggaaatctctttctgttgctggcttacagtttctctgtgtgctttgtggttgctgtcatatttgctctagaagaaaaaaaaaaaaggaggggaaatgcattttccccagagataaaggctgccattttgggggtctgtacttatggcctgaaaatatttgtgatccataactctacacagcctttactcatactattaggcacactttccccttagagccccctaagtttttcccagacgaatctttataatttcctttccaaagataccaaataaacttcagtgttttcatctaattctcttaaagttgatatcttaatattttgtgttgatcattatttccattcttaatgtgaaaaaaagtaattatttatacttattataaaaagtatttgaaatttgcacatttaattgtccctaatagaaagccacctattctttgttggatttcttcaagtttttctaaataaatgtaacttttcacaagagtcaacattaaaaaataaattattt

SEQ ID NO: 2 - protein, 790 aa, Homo sapiens
MAEQVLPQALYLSNMRKAVKIRERTPEDIFKPTNGIIHHFKTMHRYTLEMFRTCQFCPQFREIIHKALIDRNIQATLESQKKLNWCREVRKLVALKTNGDGNCLMHATSQYMWGVQDTDLVLRKALFSTLKETDTRNFKFRWQLESLKSQEFVETGLCYDTRNWNDEWDNLIKMASTDTPMARSGLQYNSLEEIHIFVLCNILRRPIIVISDKMLRSLESGSNFAPLKVGGIYLPLHWPAQECYRYPIVLGYDSHHFVPLVTLKDSGPEIRAVPLVNRDRGRFEDLKVHFLTDPENEMKEKLLKEYLMVIEIPVQGWDHGTTHLINAAKLDEANLPKEINLVDDYFELVQHEYKKWQENSEQGRREGHAQNPMEPSVPQLSLMDVKCETPNCPFFMSVNTQPLCHECSERRQKNQNKLPKLNSKPGPEGLPGMALGASRGEAYEPLAWNPEESTGGPHSAPPTAPSPFLFSETTAMKCRSPGCPFTLNVQHNGFCERCHNARQLHASHAPDHTRHLDPGKCQACLQDVTRTFNGICSTCFKRTTAEASSSLSTSLPPSCHQRSKSDPSRLVRSPSPHSCHRAGNDAPAGCLSQAARTPGDRTGTSKCRKAGCVYFGTPENKGFCTLCFIEYRENKHFAAASGKVSPTASRFQNTIPCLGRECGTLGSTMFEGYCQKCFIEAQNQRFHEAKRTEEQLRSSQRRDVPRTTQSTSRPKCARASCKNILACRSEELCMECQHPNQRMGPGAHRGEPAPEDPPKQRCRAPACDHFGNAKCNGYCNECFQFKQMYG

SEQ ID NO: 3 - DNA, 2328 bp, Mus musculus
  CDS (1)..(2328)
atggctgaacaacttcttcctcaggctttgtatttgagcaatatgcggaaagctgtgaagatacgagagagaaccccagaagacattttcaaacctaccaatgggatcatctatcactttaaaaccatgcaccgatacacgctggagatgttcagaacatgccagttttgcccacagttccgagagatcatccacaaagcacttattgacagaagtgtccaggcttccctggaaagccagaagaagctcaactggtgtcgtgaagtcaggaagctcgtggctctgaaaaccaatggtgatggaaactgcctcatgcatgcagcttgtcagtacatgtggggtgttcaggatactgacctggtcctgaggaaggccctctgcagcacccttaaggagacagacactcggaactttaaattccgctggcagctggaatctctgaaatctcaggaatttgtggaaacaggactttgctacgacactcggaactggaatgacgaatgggacaacttggtcaaaatggcatcagcagacacacctgcagcccgaagtggacttcagtacaattccctggaagaaatccacatatttgtcctcagcaacatcctcagaagacccatcattgtcatttcagacaaaatgctaagaagtttggaatctggttccaattttgctcctttgaaagtgggtgggatttatctgcctcttcactggcctgcccaggagtgttacagatatcccatcgtcctaggctatgacagccagcactttgtacccctggtgaccctgaaggacagtggacctgaacttcgcgctgttccacttgttaacagagaccggggtaggtttgaagacttaaaagttcacttcttgacagatcctgagaatgagatgaaggaaaagcttctaaaggagtacttgatagtgatggagatccctgtgcaaggctgggaccacggcacgactcacctgatcaacgctgcaaaattggatgaagctaacttacccaaagaaataaatttggtagacgattactttgagcttgttcagcacgaatacaagaaatggcaggagaacagcgatcaggccaggagagcggcacatgcgcagaaccccttggagccttccacaccccagctatcactcatggatataaaatgtgagacacccaactgtcctttcttcatgtccgtgaacactcagcctttatgccacgaatgctcagagaggcgccaaaagaatcagagcaagctcccaaagctgaactcgaagctaggccctgaaggactcccaggcgtgggacttggctcctcaaactggagccccgaggaaaccgctggaggacctcattcagccccacccacagcacccagcctttttctcttcagtgagaccactgcaatgaagtgcaggagtcctgggtgcccttttactttgaatgtgcagcataatggattctgtgagcgttgccacgcccggcagattaatgccagccacaccgcagaccctggaaagtgccaagcctgccttcaggatgtcactcggacctttaatggcatctgcagtacctgtttcaaaaggactacagcagagcccagctccagcctcacttccagtatccctgcctcctgtcaccaacgctccaagtctgacccctcacaactcatccaaagtctcactccacactcttgccaccggactggaaatgtctctccttctggctgcctctcccaggctgcacggactccaggagacagagcagggacaagcaagtgcaggaaagctggctgcatgtattttgggactccagaaaacaagggcttttgcactctatgtttcatcgaatacagagaaaataagcagtctgttactgcctctgcgaaagctggttccccggcccccaggttccagaacaatgtcccgtgcctgggcagggagtgcggcacactcggaagcaccatgtttgaagggtactgtcagaagtgtttcatcgaagctcagaaccagagattccatgaagcaagaagaacggaagaacagctgagatcaagccagcatagagacatgcctcgaactacacaggtagcctcaaggctgaaatgtgcccgggcctcctgcaagaacattctggcctgtcgcagtgaggaactctgtatggagtgccagcacctaagccaacgagtaggttctgtggcccaccggggtgagcccacgcctgaagagccccctaaacagcgctgccgggcccctgcttgtgatcactttggcaatgccaagtgtaatggttactgcaatgagtgctaccagttcaagcagatgtatggctaa

SEQ ID NO: 4 - protein, 775 aa, Mus musculus
MAEQLLPQALYLSNMRKAVKIRERTPEDIFKPTNGIIYHFKTMHRYTLEMFRTCQFCPQFREIIHKALIDRSVQASLESQKKLNWCREVRKLVALKTNGDGNCLMHAACQYMWGVQDTDLVLRKALCSTLKETDTRNFKFRWQLESLKSQEFVETGLCYDTRNWNDEWDNLVKMASADTPAARSGLQYNSLEEIHIFVLSNILRRPIIVISDKMLRSLESGSNFAPLKVGGIYLPLHWPAQECYRYPIVLGYDSQHFVPLVTLKDSGPELRAVPLVNRDRGRFEDLKVHFLTDPENEMKEKLLKEYLIVMEIPVQGWDHGTTHLINAAKLDEANLPKEINLVDDYFELVQHEYKKWQENSDQARRAAHAQNPLEPSTPQLSLMDIKCETPNCPFFMSVNTQPLCHECSERRQKNQSKLPKLNSKLGPEGLPGVGLGSSNWSPEETAGGPHSAPPTAPSLFLFSETTAMKCRSPGCPFTLNVQHNGFCERCHARQINASHTADPGKCQACLQDVTRTFNGICSTCFKRTTAEPSSSLTSSIPASCHQRSKSDPSQLIQSLTPHSCHRTGNVSPSGCLSQAARTPGDRAGTSKCRKAGCMYFGTPENKGFCTLCFIEYRENKQSVTASAKAGSPAPRFQNNVPCLGRECGTLGSTMFEGYCQKCFIEAQNQRFHEARRTEEQLRSSQHRDMPRTTQVASRLKCARASCKNILACRSEELCMECQHLSQRVGSVAHRGEPTPEEPPKQRCRAPACDHFGNAKCNGYCNECYQFKQMYG

(19) United States
(12) Patent Application Publication
(10) Pub. No.: US 2003/0171253 A1
(43) Pub. Date: Sep. 11, 2003

(54) Methods and compositions relating to modulation of A20

(76) Inventors: Averil Ma, Chicago, IL (US); David Boone, Chicago, IL (US); Eric Lee, Torrance, CA (US)

Correspondence Address: Robert E. Hanson, Fulbright & Jaworski L.L.P., Suite 2400, 600 Congress Avenue, Austin, TX, 78701, US

(21) Appl. No.: 10/125,770

(22) Filed: Apr. 18, 2002

Related U.S. Application Data
(60) Provisional application No. 60/285,427, filed on Apr. 19, 2001.

(51) Int. Cl. A61K 31/00; G01N 33/574

(52) U.S. Cl. 514/1; 435/7.23

(57) ABSTRACT

The invention provides compositions and methods for treating diseases characterized by aberrant programmed cell death and/or inflammation, comprising mediating A20 function in the subject. Such diseases include Crohn's disease, inflammatory bowel disease, a disease associated with ischemic injury, a toxin-induced liver disease and cancer. The invention further provides methods and compositions for assays for modulators of A20.

This application claims the priority of U.S. Provisional Patent Application Serial No. 60/285,427, filed Apr. 19, 2001, the entire disclosure of which is specifically incorporated herein by reference.

### BACKGROUND OF THE INVENTION

1. Field of the Invention

The present invention relates generally to the field of medicine. More particularly, it concerns compositions and methods for treating disease conditions comprising modulating A20.

2. Description of Related Art

Intestinal inflammatory responses to microbial pathogens must be delicately balanced to effectively eliminate pathogens while preventing autoimmunity. The critical roles of cytokines in maintaining this balance have been demonstrated in both experimental models such as gene targeted transgenic mice and in human IBD patients. (Sadlock et al., 1993; Willerford et al., 1995; Kuhn et al., 1993; Blumberg et al., 1999; Fiocchi, 1999). One of the most important cytokines for regulating intestinal inflammation is tumor necrosis factor (TNF). The critical roles of TNF in this regard are highlighted both by animal studies showing that TNF overexpression by targeted deletion of TNF 3′ untranslated mRNA stability elements leads to ileitis and arthritis (TNF^{ΔARE} mice) (Kontoyiannis et al., 1999), and by human studies and clinical experience showing that IBD patients express high levels of TNF and frequently respond to anti-TNF therapy (Targan et al., 1997; Rutgeerts et al., 1999). These observations indicate that TNF is a critical regulator of immune homeostasis.

TNF is elaborated from macrophages as well as multiple other cell types, in response to stimuli such as IL-1, LPS and TNF itself. TNF binds to two TNF receptors, TNFR1 and TNFR2, stimulating several signaling pathways, including NF-κB, JNK and caspase mediated programmed cell death (PCD) pathways (Chan et al., 2000). Binding of TNF to TNFR1 and TNFR2 activates several proteins, including the RIP kinase, which in turn leads to activation of the inhibitor of kinase kinase (IKK) complex, comprised of IKKα, IKKβ and IKKγ. IKK phosphorylates IκBα, which is then degraded by proteasomes and releases active NF-κB to traverse to the nucleus and mediate transcription of NF-κB target genes (Karin and Ben-Neriah, 2000). Many of these genes are pro-inflammatory genes, such as TNF, IFN-γ, IL-1, IL-6, IL-8, IL-12, MCP-1, P-selectin, E-selectin, and iNOS. The increased expression of these proteins facilitates inflammatory reactions by supporting the activation and differentiation of immune cells, recruiting additional immune cells to sites of inflammation, facilitating the passage of immune cells from the circulation across endothelial cells into inflamed tissues, and stimulating the elaboration of additional proinflammatory factors. Thus, TNF mediates pro-inflammatory cascades via NF-κB activation. The importance of NF-κB activation to human disease is highlighted by observations that NF-κB dependent cytokines are elevated in human inflammatory bowel disease patients (Neurath et al., 1998). Thus, persistent TNF and NF-κB activity are associated with conditions such as bowel inflammation in both experimental models and human disease.

The ability of newly synthesized TNF to further amplify inflammatory responses indicates that the regulation of cellular responses to TNF must be as carefully regulated as the elaboration of TNF to maintain immune homeostasis. Many cellular responses to TNF result from activating the transcription factor NF-κB. Activation of NF-κB dependent transcriptional activity leads to the synthesis of genes encoding IL-1, IL-12, TNF and IFN-γ in macrophages, iNOS in granulocytes, IL-2 receptor alpha (IL-2Rα) in T cells, and P-selectin and E-selectin in endothelial cells. Thus, regulation of TNF induced inflammatory responses is largely dependent upon the regulation of NF-κB activity. While the biochemical steps leading to NF-κB signaling activity have been intensely studied, less is known about how these pathways are terminated or how cellular responses to TNF are down-regulated (Karin, 1999). Termination of NF-κB activity is thought to occur because IκBα, the protein that normally retains NF-κB in an inactive state in the cytoplasm, is itself an NF-κB responsive gene (Sun et al., 1993). NF-κB activity in the nucleus leads to de novo synthesis of IκBα, which then binds to NF-κB and inactivates NF-κB. Indeed, the inability to inhibit NF-κB activity results in spontaneous inflammation in mice that lack the constitutive inhibitor of NF-κB, IκBα (Beg et al., 1995). Thus, IκBα represents a critical terminator of NF-κB activity. However, it is less clear why stimulated cells do not persistently induce NF-κB signals by re-phosphorylating de novo synthesized IκBα and thus re-activating NF-κB. Similarly, it is unclear why cells become refractory to repeated stimulation by TNF.

Activation of immune cells is followed rapidly by cellular proliferation and expansion of these cells. Entry into cell cycle is typically initiated by the activation of c-jun pathway signaling and AP-1 dependent transcription of genes such as c-myc, cyclin D and c-jun itself. In addition to activating NF-κB, TNFR signals activate JNK pathway signals leading directly to c-jun phosphorylation. Thus, TNF can synergistically activate both cellular activation and proliferation. As with NF-κB signaling, many studies have examined the biochemical events involved in activation of JNK signaling, but few studies have examined what events terminate JNK signaling. While dephosphorylation and/or ubiquitin dependent degradation of relevant kinases may be important in inactivating signals to c-jun, the critical steps in termination of JNK signaling are poorly understood. The importance of negative regulating these signals is highlighted by the presence of excessive c-Jun activity, leading to granulocytosis and leukemia in mice lacking the c-jun inhibitor, jun B (Passegue et al., 2001). Recent work has suggested that NF-κB dependent gene products may help ultimately terminate JNK pathway signaling in fibroblast cell lines (Tang et al., 2001; De Smaele et al., 2001). However, it is uncertain which proteins mediate this cross-talk between signaling pathways, and whether such cross-talk occurs in innate immune cells. Given the strong associations of TNF, JNK and NF-κB activity with inflammation, properly regulated termination of these signaling pathways is likely to be critical for limiting inflammation in vivo.

As most cells in the body—including both immune and non-immune cells—express TNFRs, controlling TNF induced NF-κB activity is likely to be important for multiple facets of inflammation. For example, active NF-κB leads to the synthesis of IL-1, IL-12, TNF and IFN-γ in macrophages, IL-2 receptor alpha (IL-2Rα) in T cells, IL-8 in epithelial cells, and P-selectin and E-selectin in endothelial cells (Schmid and Adler, 2000). As many of these proteins mediate intercellular signals, dysregulated NF-κB activity of one cell type may perturb the activity of multiple other cell types. Thus, dissecting the mechanisms by which each cell type regulates TNF and NF-κB activity will be important for fully understanding which cells are pathogenically important for regulating inflammation in a complex tissue such as the intestine.

In addition to stimulating NF-κB activity, TNF binding to TNFR1 can also induce PCD. TNFR engagement can recruit TRADD and FADD proteins to the receptor, ultimately resulting in activation of caspases and death of the cell. This pathway can be unveiled when protein synthesis or NF-κB activity is experimentally blocked. While most cells do not undergo PCD when exposed to TNF alone in vitro (Beg, 1996; Wang et al., 1996), stressful physiological conditions (e.g., inflammation) could theoretically restrict protein synthesis and predispose cells to TNF induced PCD in vivo. Such physiological PCD caused by TNF might facilitate removal of damaged or infected cells during inflammatory responses. In this context, the proper regulation of TNF induced signals may be critical for limiting tissue damage in the environment of an inflammatory reaction.

### SUMMARY OF THE INVENTION

The inventors have investigated the physiological roles of A20, a lymphoid specific molecule which can inhibit TNF induced NF-κB and PCD in cell lines in vitro (Opipari et al., 1990; Tewari et al., 1995; Song et al., 1996). A20 is a cytoplasmic zinc finger protein which is induced, for example, by TNF and can interact with a variety of proteins. By generating and characterizing A20 deficient mice, the inventors have found that A20 is dramatically induced in multiple tissues and is essential for terminating both spontaneous and TNF induced inflammation in vivo. However, the inventors have also shown that A20 has a role in potentiating inflammatory stimuli other than 1F. This finding demonstrates, for example, that inflammation is normally held in check by A20 and that A20 also protects stromal cells against deleterious side effects from stimuli including TNF.

The present invention relates, in some aspects, to methods of treating a subject a subject for a disease characterized by aberrant levels of programmed cell death and/or inflammation, comprising mediating A20 function in the subject. The subject may preferentially be a mammal, for example a rodent, mouse, or human. In many cases, the subject has or is at risk of having a disease. In one embodiment of the invention, the disease is selected from the group consisting of Crohn's disease, heart failure, inflammatory bowel disease, arthritis, diabetes, pulmonary inflammation, nephritis, a vascular disease mediated by endothelial cell dysfunction, a disease associated with ischemic injury, a toxin-induced liver disease and cancer. The disease may also be associated with ischemic injury, including tissue ischemia. Ischemic injury includes, for example, necrosis. The disease may further be septic shock and the toxin-induced liver disease may be cirrhosis of the liver. In certain embodiments of the invention, the disease is cancer, including non-Hodgkins lymphoma. In still further embodiments, the disease is pulmonary inflammation or nephritis

Mediating A20 function can comprise providing an A20 polypeptide or a modulator of A20 activity to the subject. The provision may comprise formulating the A20 polypeptide or modulator of A20 in a pharmaceutical composition, which may then be administered to the subject. Such compositions are described herein below. The administration may be accomplished by any method disclosed herein. In some preferred embodiments, the administration comprises injection of an A20 polypeptide into the subject. The provision of the 20 polypeptide can comprise obtaining an A20 polypeptide and incorporating it into a pharmaceutical carrier. Alternatively, in some examples, providing an A20 polypeptide comprises providing a nucleic acid segment encoding the A20 polypeptide to the subject and obtaining expression of the polypeptide in the subject. The A20 polypeptide may be a modified A20 polypeptide prepared as described elsewhere in the application and known to those of skill in the art.

In other embodiments, mediating A20 function comprises providing a modulator of A20 function to the subject. The modulator of A20 function may be further defined as comprising screening for a modulator of A20 function. After screening, one can determine a modulator of A20 function and providing that modulator to the subject. The modulator can b, for example, is an agonist or antagonist of A20. The modulator may modulate a protease activity of A20. The modulator can be a polypeptide, for example a protease inhibitor. The modulator can be a nucleic acid segment. The nucleic acid segment can encode an A20 polypeptide, a polypeptide modulator of A20 activity. The nucleic acid may be an antisense nucleic acid to a nucleic acid encoding A20. The modulator may be a small molecule, for example, a protease inhibitor or agonist of A20.

Modulating A20 function may comprise modulating A20 concentration in the subject. For example, A20 concentration my be increased or decreased. Modulating A20 concentration may comprise modulating A20 expression in the subject. Modulating A20 expression, in some cases, comprises modulating A20 transcription and/or A20 translation. In some cases, modulating A20 concentration comprises modulating the half-life of A20 in the subject. For example, the half-life of A20 can be increased.

The invention relates, in some embodiments, to methods of modulating a TNF mediated pathway, for example, an NF-κB pathway, a JNK pathway, or a programmed cell death pathway. In certain embodiments of the invention, A20 may be used to regulate inflammatory stimuli including, but not limited to, TNF, IL-1 and LPS. In accordance with the invention, regulation of the response to other stimuli may further be carried out.

The inventors have further noted that A20 has a protease domain that precisely matches a sequence in the HIV Ned protein necessary for viral disease. It is thus noted by the inventors that modulation of A20 activity may find use in the treatment of a subject for HIV infection. Such modulation may be carried out as is described herein below in detail.

In some cases, the invention envisions methods of modulating an immune response. For example, the invention allows one to inhibit or induce an immune response. Inhibition of an immune response may comprise inhibiting TNF activity. Some specific embodiments comprise inhibiting TNF induced NF-κB activity, sometimes also resulting inhibition of TNF induced programmed cell death. Some methods involve inhibiting both NF-κB activity and programmed cell death. The methods of the invention are, in some aspects, useful to prevent or treat a disease state involving an immune response. Such a disease state may result from a disease effecting the digestive tract, such as inflammatory bowel disease or Crohn's disease. It is possible to increase A20 activity, for example, to induce an immune response. As such, the invention relates to methods of preventing or treating a disease state involving a lack of an immune response.

The invention also contemplates methods of modulating programmed cell death, for example, methods of modulating TNF induced programmed cell death. Such methods may result in increasing or decreasing programmed cell death. As such, these methods are useful to the treatment of cancer.

In some specific embodiments, the invention relates to methods of treating or preventing TNF induced inflammation in a subject comprising mediating A20 function in the subject. In other specific embodiments, the invention relates to methods of modulating TNF induced programmed cell death in a subject comprising modulating A20 activity in the subject. In still further embodiments of the invention, methods are provided of treating a subject for Crohn's disease and/or inflammatory bowel disease, comprising increasing A20 function in the subject.

Based on data obtained by the inventors, the invention contemplates methods of modulating A20 activity to modulate TNF mediated activity without modulating IL-1 mediated activity, modulate myeloid cells, and/or modulate granulocytes.

The invention also contemplates a mammal that is homozygous negative for an A20 allele. In some cases, the mammal is further defined as a mouse. The invention further relates to methods of producing a transgenic mouse that is homozygous negative for an A20 allele.

Certain further aspects of the invention provide methods of isolating modulators of A20 function comprising screening with a mammal that is homozygous negative for an A20 allele. In some embodiments, the mammal is a mouse. For example, the invention includes methods of screening for modulators of an A20-mediated process comprising obtaining a candidate substance and contacting a subject that is homozygous for an A20 negative allele with the candidate substance.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. A20 provides negative feedback of TNF induced NF-κB activity.

FIG. 2. Generation of A20 deficient mice. A gene targeting construct is shown which is designed to eliminate the ATG start codon and the first 738 base pairs of the coding sequence of A20.

FIG. 3. Gross appearance of four week old A20^{+/+} and A20^{-/-} mice.

FIG. 4. Gross appearance of A20^{+/+} and A20^{-/-} livers. Note pale acellular regions of A20^{-/-} livers.

FIG. 5. Hematoxylin and eosin (H&E) stained sections from A20^{+/+} and A20^{-/-} livers. Note inflammation and hepatocyte loss in A20^{-/-} livers.

FIG. 6. Gross appearance of A20^{+/+} and A20^{-/-} kidneys. Note atrophied kidney in A20^{-/-} mouse.

FIG. 7. H&E kidney sections. Note interstitial nephritis, glomerular dilatation and cortical tubular atrophy in small A20^{-/-} kidney.

FIG. 8. H&E colonic sections. Note colitis, including lamina propria inflammation, crypt abscess, and branching epithelial crypts in A20^{-/-} colon.

FIG. 9. H&E joint and bone sections. Note bone marrow replacement with inflammatory cells, thinned trabecular bone, and destructive arthritis in A20^{-/-} bone and joint.

FIG. 10. H&E kidney sections from A20^{±} RAG-1^{-/-} (left panel) and A20^{-/-} RAG-1^{-/-} (right panel) mice. Note normal appearance of A20^{±} RAG-1^{-/-} kidney. Note interstitial nephritis, glomerular dilatation and cortical tubular atrophy in A20^{-/-} RAG-1^{-/-} kidney (right panel), comparable to A20^{-/-} RAG-1^{+/+} kidney (FIG. 12 right panel).

FIG. 11. Aberrant dermal differentiation. Note thickened epidermis and dermis, and loss of hair follicles and fat in A20^{-/-} skin.

FIG. 12. Induction of A20 expression in vivo. Northern analysis of A20 mRNA expression in tissues is shown from TNF injected normal mice (LIV=liver; KID=kidney; SPL=spleen; THY=thymus; COL=colon; LN=lymph node). Comparable RNA loading and integrity confirmed by ethidium staining of 28S rRNA.

FIG. 13. Sensitivity to TNF induced PCD in A20^{-/-} thymocytes. Survival is shown of A20^{-/-} (solid bars) and A20^{±} (hatched bars) thymocytes from two to three week old mice five hours after in vitro treatment with the indicated agents. TNF used at 10 ng/ml and cycloheximide (CHX) used at 10 μg/ml in all studies. * indicates p<0.001 by Tukey's test.

FIG. 14 and FIG. 15. Western analyses of IκBα (Santa Cruz Biotechnology, SCB), phospho-SAPK/JNK (New England Biolabs, NEB), SAPK/JNK (NEB), Bcl-x (Transduction Labs) and Bcl-2 (Pharmingen) proteins in lysates from TNF treated thymocytes.

FIG. 16. TNFα induced PCD in A20^{-/-} MEFs for CHX, TNF+CHX and TNF→TNF+CHX.

FIG. 17. Western analyses of TRAF2 (MedBiol Labs), cIAP-1 (Trevigen), phospho-SAPK/JNK and SAPK/JNK proteins in lysates from TNF treated MEFs.

FIG. 18. Critical role for A20 in terminating TNF induced NF-kB signals. Shows EMSA analyses of NF-κB activity, using an NF-κB consensus oligonucleotide (SCB).

FIG. 19. Hypersensitivity of A20^{-/-} intestinal epithelium to TNF. Shows damaged A20^{-/-} intestinal epithelium after TNF injection. Note dramatic loss of epithelial integrity after TNF injection in A20^{-/-} mouse. Mag.=100× or 400×

FIG. 20. Western blot analysis of IκBα expression.

FIG. 21. Northern blot analyses of IκBα and glyceraldehyde phosphate dehydrogenase (GAPDH) mRNA expression in MEFs.

FIG. 22A and FIG. 22B. Western blot analyses of IκBα and phospho-IκBα expression after proteasome inhibition (FIG. 22A). IKK kinase assay of TNF treated MEFs. Total cell lysates from repeatedly TNF treated MEFs were immunoprecipitated with an anti-IKKγ antibody (SCB), and kinase activity was assessed using a GST-IκBα (1-54) substrate (FIG. 22B upper panels). Comparable IKKβγ protein in immunoprecipitated samples confirmed by Western analysis (FIG. 22B lower panels).

FIG. 23. Western analysis of IκBα expression in IL-1βγ treated MEFs.

FIG. 24. Spontaneous colitis in A20^{-/-} RAG-1^{-/-} double mutant mice. Thickened, inflamed A20^{-/-} RAG-1^{-/-} colon (right)with mononuclear infiltrate and distorted crypt morphology, compared with normal colon (left). Indicates that A20 is critical for regulating inflammation even in the complete absence of lymphocytes. Mag.=100× for both specimens.

FIG. 25. Colitis in RAG-1^{-/-} mice reconstituted with A20^{-/-} fetal liver. Thickened, inflamed colon from RAG-1^{-/-} mouse reconstituted with A20^{-/-} (right) but not A20^{+/+} (left) fetal liver hematopoietic cells. Indicates that A20 regulates hematopoietic cells even when endothelial cells and other stromal cells are normal and demonstrates the importance of A20 in a model of a human inflammatory bowel disease in humans (colitis). Mag.=100× for both specimens.

FIG. 26. Increased numbers of activated (CD44^{Hi}) CD4⁺ (right) and CD8⁺ (left) T cells in A20^{-/-} mice.

FIG. 27. Increased numbers of activated Mac-1⁺ Gr-1^{Hi} cells (granulocytes) and Mac-1+Gr-1^{Int} (macrophages) in tissues from A20^{-/-} mice.

FIG. 28. A20 protein expression in purified mature T and B cells (left panels), and thymocytes (right panels). 82 kD band specific for A20 indicated on blots.

FIG. 29. RNAse protection analysis of intestinal tissue from A20^{+/+} RAG-1^{+/+}, A20^{-/-} RAG-1^{+/+} and A20^{-/-} RAG-1^{-/-} mice. Note increased expression of LTβ, TNFα, IFNγ, and TGFβ1 in A20^{-/-} RAG-1^{+/+} mice, while only TNFα is increased in A20^{-/-} RAG-1^{-/-} mice.

FIG. 30. Flow cytometric analysis of donor Ly5. 1⁺ A20^{+/+} (left panels) or A20^{-/-} (right panels) cells from spleen and liver tissues from chimeric Ly5.2 recipient mice.

FIG. 31. Increased numbers of BrdU⁺ macrophages in tissues from chimeric A20^{-/-} mice.

FIG. 32. Increased numbers of donor granulocytes and macrophages in spleens from chimeric mice reconstituted with A20^{-/-} RAG-1^{-/-} (black) compared with A20^{±} RAG-1^{-/-} (hatched) stem cells.

FIG. 33. Elevated and prolonged P-JNK levels after TNF treatment in A20^{-/-} thymocytes.

FIG. 34. Elevated and prolonged c-jun kinase activity after TNF treatment in A20^{-/-} thymocytes (middle panels=KA=kinase assay).

FIG. 35. Prolonged P-Jun levels after TNF treatment in A20^{-/-} (as compared with A20^{+/+}) thymocytes.

FIG. 36. Prolonged c-jun levels after TNF treatment in A20^{-/-} (as compared with A20^{+/+}) thymocytes.

FIG. 37. Western blot analysis of A20^{+/+} (left ten panels) and A20^{-/-} (right two panels) MEFs treated with TNF for various times. Note induction of A20 protein in A20^{+/+} MEFs.

### DESCRIPTION OF ILLUSTRATIVE EMBODIMENTS

The inventors have overcome the limitations of the prior art by elucidating the cellular roles of A20. The inventors have thus identified A20 as a critical molecule in, for example, inhibiting inflammation. The invention further allows modulation of A20 for the treatment of other disease conditions including, for example, diseases associated with ischemic injury, toxin-induced liver disease and cancer. By generating and characterizing A20^{-/-} mice, the inventors have obtained study data strongly suggesting that A20 is essential for protecting mice from both spontaneous and TNF induced inflammation. The importance of A20 to immune homeostasis has never previously been appreciated. The dramatic histologic findings obtained thus far indicate that the regulation of cellular responses to TNFR signals is a critical aspect of immune regulation.

The inventors have also begun molecular investigations indicating that A20 is the first intracellular signaling protein known that inhibits TNF induced NF-κB activity and TNF induced PCD in vivo. These studies will be extended to dissecting the role of A20 in regulating other pro-inflammatory and PCD signals as well. The data have significance to the role of A20 in: (i) regulating TNF responses in different hematopoietic and non-hematopoietic cell types in the intestine; (ii) regulating TNF induced NF-κB versus TNF induced PCD responses in these cells; and (iii) regulating signals from different TNFR receptor family members in vivo. The studies highlight the importance of properly regulating the cellular responses to TNF as well. Given the success of anti-TNF antibody therapies in humans, the studies may lead to novel and synergistic strategies for treating IBD and other conditions, as described herein, in human patients.

During inflammatory responses, TNF and interleukin-1 (IL-1) signals activate NF-κB, which regulates the transcription of other proinflammatory genes. The factors which limit these responses are poorly understood. A20 is a cytoplasmic protein thought to be expressed predominantly in lymphoid tissues, and heterologously expressed A20 can inhibit TNF induced NF-κB and PCD responses in cell lines (Opipari et al, 1992; Cooper et al, 1996; Opipari et al., 1990; Tewari et al., 1995). A20 binding to TNF receptor associated factor-2 (TRAF2), inhibitor of NF-κB kinase gamma (IKKγ), and/or A20 binding inhibitor of NF-κB activation (ABIN) suggest potential mechanisms by which A20 could regulate TNF receptor signals (Song et al., 1996; Zhang et al., 2000; Heyninck et al., 1999), however A20's functions in vivo are unknown. Thus, the inventors generated A20 deficient (A20^{-/-}) mice by gene targeting.

The inventors have found that A20, a previously understudied molecule, is required for terminating TNF induced NF-κB responses, protecting cells from TNF induced PCD, and is absolutely essential for mucosal immune homeostasis. The inventors have shown that A20 is a dynamically regulated and pleiotropically expressed gene which is required for negatively regulating NF-κB responses in vivo. A20 may also regulate TNF induced SAPK/JNK and PCD responses. A20's ability to inhibit TNF but not IL-1β induced NF-κB signals suggests these signals can be differentially regulated in vivo. The rapid expression of A20 is essential for limiting inflammatory responses and the damage those responses cause in multiple tissues.

A20 cDNA codes for a protein of 790 amino acid residues (Opipari et al., 1992). A20 contains seven novel zinc fingers which has been shown to mediate self-association in A20. The zinc fingers also mediate IL-1-induced NF-kappaB activation. Immunologicalization studies have shown A20 to be a cytoplasminc protein. Several isoforms of the 14-3-3 proteins were found to interact with A20 in a yeast two-hybrid screen (Vincenz et al., 1996).

A20, and A20 polypeptides regulate the activation kinetics of the NF-kB pathway. The activity of NF-kB depends on release of the factor from an inhibitory complex in the cytoplasm and translocation to the nucleus. A20 expression results in a marked decrease in the kinase activity of a high molecular weight complex responsible for the phosphorylation of the inhibitory proteins known as the IkBs., which results in either total blockade of the pathway or a more rapid decay of the active complexes in the nucleus. This is in support of a model of A20 action wherein A20 effects on key proteins in the high molecular weight activating complex. (http://www.scripps.-edu/research/sr99/immgen1.html).

### I. A20, TNF and Innate Immune Responses

Tumor necrosis factor (TNF) is the founding member of a highly conserved family of genes that regulates both the expansion and contraction of immune responses (Locksley et al., 2001). During the initial response to pathogens, characterized by cellular activation and proliferation, TNF is elaborated from macrophages and multiple other cell types in response to stimuli such as pathogen associated microbial motifs. TNF binds to two TNF receptors, TNFR1 and TNFR2, that are expressed on a wide array of cell types. Consequently, TNF activates multiple cell types, including innate and adaptive immune cells, endothelial and stromal cells. Activation of innate immune cells such as macrophages, dendritic cells and granulocytes leads to the elaboration of multiple additional pro-inflammatory cytokines and effector molecules (e.g., nitric oxide and IL-12) as well as the expression of surface membrane co-stimulatory molecules (e.g., B7, CD40). In addition, activation of endothelial cells facilitates the passage of immune cells from the circulation across endothelial cells into inflamed tissues, and activation of stromal cells leads to secretion of chemokines, thus recruiting inflammatory cells to the site of pathogens. In these ways, TNF plays pleiotropic roles in mediating inflammation.

The critical roles of TNF are highlighted by studies in experimental models showing that TNF over-expression leads to multi-organ autoimmunity (TNF^{ΔARE} mice) (Kontoyiannis et al., 1999). Given the large number of cell types that respond to TNF, it is not surprising that autoimmunity in such models occurs via both lymphocyte-dependent and lymphocyte-independent mechanisms. In this regard, innate immune cells mediate inflammation by both directly causing inflammation as well as by recruiting adaptive lymphocytes. Accordingly, one important aspect of the current invention concerns use of A20 in regulating TNF responses in innate immune cells.

To better understand how TNF signals are regulated in vivo, the inventors have focused on A20, a cytoplasmic zinc finger protein that is induced by TNF via NF-κB activation and can inhibit both TNF induced NF-κB and JNK activity (Opipari et al., 1990; Jaattela et al., 1996). The results using A20 deficient (A20^{-/-}) mice suggests that A20 is an essential regulator of innate immune cells. Moreover, the data of the inventors suggests that A20 may be the first molecule to be required for the termination of both NF-κB and JNK responses to TNF. The studies further proposed herein will demonstrate the role of A20 in regulating specific innate immune cells and elucidate the molecular mechanisms by which A20 regulates TNF responses.

The findings by the inventors that A20 deficient (A20^{-/-}) mice develop spontaneous inflammation indicate that A20 is essential for preventing uncontrolled inflammation, at least in part by terminating TNF induced NF-κB activity. The role of A20 in regulating innate immune cells is also of interest, including macrophages, granulocytes and dendritic cells in several genetic models of A20 deficiency.

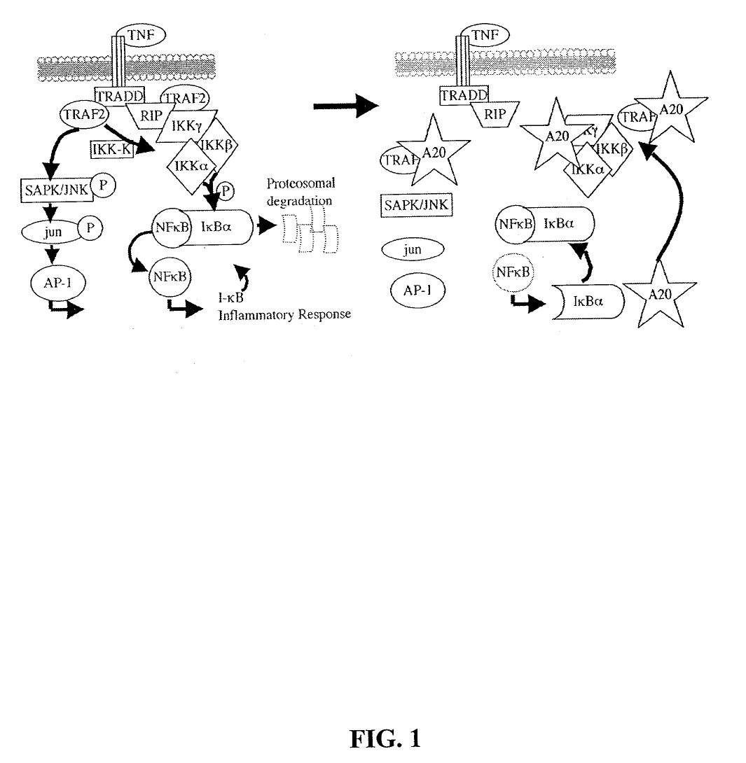

The inventors have found that A20 mRNA is dramatically induced in multiple tissues by TNF, and thus may regulate TNF signals in multiple cell types. As innate immune cells appear to be profoundly affected by A20 deficiency, these cells are central regulators of both innate and adaptive immune responses, and as genetic and cellular dissection of A20^{-/-} mice suggest that innate immune cells display cell-autonomous dependence on A20 function, the inventors have chosen to focus on the roles of A20 in regulating these cells. The inventors have obtained preliminary data suggesting that A20 regulates both the homeostasis of these cells as well as their responses to innate immune stimuli. In preliminary efforts to understand how A20 regulates these cells, the inventors have obtained preliminary data suggesting that A20 is an essential negative regulator of TNF induced NF-κB and possibly JNK responses. These findings suggest that A20 may be the second molecule identified to be essential for terminating NF-κB (after IκBα), the first molecule essential for terminating JNK signaling, and the first molecule required for terminating both NF-κB and JNK signals. Having established the novel model of A20^{-/-} mice, the inventors are currently defining the roles for A20 in regulating the behavior of innate immune cells as well as the molecular mechanisms by which A20 may regulate TNF induced NF-<B and JNK responses (FIG. 1).

### II. Screening For Modulators Of A20 Function

The present invention further comprises methods for identifying modulators of the function or activity of A20 polypeptides. These assays may comprise random screening of large libraries of candidate substances; alternatively, the assays may be used to focus on particular classes of compounds selected with an eye towards structural attributes that are believed to make them more likely to modulate the function of A20 polypeptides.

Modulation and mediation of function are used interchangeable and include the increase or decrease in the ability of an A20 polypeptide to effect the function of TNF, NF-κB or PCD activity. The modulation may similarly up- or down-regulate the JNK pathway, as is described herein Also included in the term “modulate A20 function” are a change in the transcription of A20, a change in the translation of A20, programmed cell death, or increasing the half-life of A20 polypeptide.

To identify an A20 modulator, one generally will determine the function of A20 in the presence and absence of the candidate substance, a modulator defined as any substance that alters function. For example, a method generally comprises: providing a candidate modulator; admixing the candidate modulator with an isolated compound or cell, or a suitable experimental animal; measuring one or more characteristics of the compound, cell or animal; and comparing the characteristic measured with the characteristic of the compound, cell or animal in the absence of said candidate modulator, where a difference between the measured characteristics indicates that said candidate modulator is, indeed, a modulator of the compound, cell or animal.

Assays may be conducted in cell free systems, in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

### A. Modulators

As used herein the term “candidate substance” refers to any molecule that may potentially inhibit or enhance A20 activity. The candidate substance may be a protein or fragment thereof, a small molecule, or even a nucleic acid molecule. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. For example, an antisense molecule that bound to a translational or transcriptional start site, or splice junctions, would be ideal candidate inhibitors of A20 polypeptide function.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one which exerts its inhibitory or activating effect upstream, downstream or directly on A20. Regardless of the type of inhibitor or activator identified by the present screening methods, the effect of the inhibition or activator by such a compound results in A20 as compared to that observed in the absence of the added candidate substance.

### B. In vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

### C. In cyto Assays

The present invention also contemplates the screening of compounds for their ability to modulate A20 in cells. Various cell lines can be utilized for such screening assays, including cells specifically engineered for this purpose. Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

### D. In vivo Assays

In vivo assays involve the use of various animal models, preferably transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies a modulator. The characteristics may be any of those discussed above with regard to the function of a particular compound (e.g., enzyme, receptor, hormone) or cell (e.g., growth, tumorigenicity, survival), or instead a broader indication such as behavior, anemia, immune response, etc.

The present invention provides methods of screening for a candidate substance that A20. In these embodiments, the present invention is directed to a method for determining the ability of a candidate substance to A20, generally including the steps of: administering a candidate substance to the animal; and determining the ability of the candidate substance to reduce one or more characteristics of A20.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

### III. Nucleic Acid Compositions

Certain embodiments of the present invention concern an A20 or A20 modulator nucleic acid. In certain aspects, an A20 nucleic acid comprises a wild-type or a mutant A20 nucleic acid. In particular aspects, an A20 nucleic acid encodes for or comprises a transcribed nucleic acid. In other aspects, an A20 nucleic acid comprises a nucleic acid segment of SEQ ID NO: 1 or SEQ ID NO: 3, or a biologically functional equivalent thereof In particular aspects, an A20 nucleic acid encodes a protein, polypeptide, peptide such as the human and mouse polypeptides found in SEQ ID NO: 2 and SEQ ID NO: 4.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

### A. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moeity. Preferred alkyl (e.g., alkyl, caboxyalkyl, etc.) moeities comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. A table of non-limiting, purine and pyrimidine derivatives and analogs is also provided herein below.

TABLE 1
Purine and Pyrmidine Derivatives or Analogs

| Abbr. | Modified base description |
|---|---|
| ac4c | 4-acetylcytidine |
| Chm5u | 5-(carboxyhydroxylmethyl)uridine |
| Cm | 2′-O-methylcytidine |
| Cmnm5s2u | 5-carboxymethylamino-methyl-2-thioridine |
| Cmnm5u | 5-carboxymethylaminomethyluridine |
| D | Dihydrouridine |
| Fm | 2′-O-methylpseudouridine |
| Gal q | Beta,D-galactosylqueosine |
| Gm | 2′-O-methylguanosine |
| I | Inosine |
| I6a | N6-isopentenyladenosine |
| m1a | 1-methyladenosine |
| m1f | 1-methylpseudouridine |
| m1g | 1-methylguanosine |
| m1I | 1-methylinosine |
| m22g | 2,2-dimethylguanosine |
| m2a | 2-methyladenosine |
| m2g | 2-methylguanosine |
| m3c | 3-methylcytidine |
| m5c | 5-methylcytidine |
| m6a | N6-methyladenosine |
| m7g | 7-methylguanosine |
| Mam5u | 5-methylaminomethyluridine |
| Mam5s2u | 5-methoxyaminomethyl-2-thiouridine |
| Man q | Beta,D-mannosylqueosine |
| Mcm5s2u | 5-methoxycarbonylmethyl-2-thiouridine |
| Mcm5u | 5-methoxycarbonylmethyluridine |
| Mo5u | 5-methoxyuridine |
| Ms2i6a | 2-methylthio-N6-isopentenyladenosine |
| Ms2t6a | N-((9-beta-D-ribofuranosyl-2-methylthiopurine-6-yl) |
| | carbamoyl)threonine |
| Mt6a | N-((9-beta-D-ribofuranosylpurine-6-yl)N-methyl- |
| | carbamoyl)threonine |
| Mv | Uridine-5-oxyacetic acid methylester |
| o5u | Uridine-5-oxyacetic acid (v) |
| Osyw | Wybutoxosine |
| P | Pseudouridine |
| Q | Queosine |
| s2c | 2-thiocytidine |
| s2t | 5-methyl-2-thiouridine |
| s2u | 2-thiouridine |
| s4u | 4-thiouridine |
| T | 5-methyluridine |
| t6a | N-((9-beta-D-ribofuranosylpurine-6-yl) |
| | carbamoyl)threonine |
| Tm | 2′-O-methyl-5-methyluridine |
| Um | 2′-O-methyluridine |
| Yw | Wybutosine |
| X | 3-(3-amino-3-carboxypropyl)uridine, (acp3)u |

A nucleobase may be comprised in a nucleside or nucleotide, using any chemical or natural synthesis method described herein or known to one of ordinary skill in the art.

### B. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992).

### C. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

### D. Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids comprising 5-carbon sugar and/or backbone moiety derivatives or analogs, include those in U.S. Pat. No. 5,681,947 which describes oligonucleotides comprising purine derivatives that form triple helixes with and/or prevent expression of dsDNA; U.S. Pat. Nos. 5,652,099 and 5,763,167 which describe nucleic acids incorporating fluorescent analogs of nucleosides found in DNA or RNA, particularly for use as flourescent nucleic acids probes; U.S. Pat. No. 5,614,617 which describes oligonucleotide analogs with substitutions on pyrimidine rings that possess enhanced nuclease stability; U.S. Pat. Nos. 5,670,663, 5,872,232 and 5,859,221 which describe oligonucleotide analogs with modified 5-carbon sugars (i.e., modified 2′-deoxyfuranosyl moieties) used in nucleic acid detection; U.S. Pat. No. 5,446,137 which describes oligonucleotides comprising at least one 5-carbon sugar moiety substituted at the 4′ position with a substituent other than hydrogen that can be used in hybridization assays; U.S. Pat. No. 5,886,165 which describes oligonucleotides with both deoxyribonucleotides with 3′-5′ internucleotide linkages and ribonucleotides with 2′-5′ internucleotide linkages; U.S. Pat. No. 5,714,606 which describes a modified internucleotide linkage wherein a 3′-position oxygen of the internucleotide linkage is replaced by a carbon to enhance the nuclease resistance of nucleic acids; U.S. Pat. No. 5,672,697 which describes oligonucleotides containing one or more 5′ methylene phosphonate internucleotide linkages that enhance nuclease resistance; U.S. Pat. Nos. 5,466,786 and 5,792,847 which describe the linkage of a substituent moeity which may comprise a drug or label to the 2′ carbon of an oligonucleotide to provide enhanced nuclease stability and ability to deliver drugs or detection moieties; U.S. Pat. No. 5,223,618 which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967 which describes oligonucleotides comprising at least one sulfamate or sulfamide internucleotide linkage that are useful as nucleic acid hybridization probe; U.S. Pat. No. 5,378,825, 5,777,092, 5,623,070, 5,610,289 and 5,602,240 which describe oligonucleotides with three or four atom linker moeity replacing phosphodiester backbone moeity used for improved nuclease resistance, cellular uptake and regulating RNA expression; U.S. Pat. No. 5,858,988 which describes hydrophobic carrier agent attached to the 2′-O position of oligonuceotides to enhanced their membrane permeability and stability; U.S. Pat. No. 5,214,136 which describes olignucleotides conjugaged to anthraquinone at the 5′ terminus that possess enhanced hybridization to DNA or RNA; enhanced stability to nucleases; U.S. Pat. No. 5,700,922 which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154 which describes RNA linked to a DNA to form a DNA-RNA hybrid.

### E. Polyether and Peptide Nucleic Acids

In certain embodiments, it is contemplated that a nucleic acid comprising a derivative or analog of a nucleoside or nucleotide may be used in the methods and compositions of the invention. A non-limiting example is a “polyether nucleic acid”, described in U.S. Pat. No. 5,908,845, incorporated herein by reference. In a polyether nucleic acid, one or more nucleobases are linked to chiral carbon atoms in a polyether backbone.

Another non-limiting example is a “peptide nucleic acid”, also known as a “PNA”, “peptide-based nucleic acid analog” or “PENAM”, described in U.S. Pat. Nos. 5,786,461, 5,891,625, 5,773,571, 5,766,855, 5,736,336, 5,719,262, 5,714,331, 5,539,082, and WO 92/20702, each of which is incorporated herein by reference. Peptide nucleic acids generally have enhanced sequence specificity, binding properties, and resistance to enzymatic degradation in comparison to molecules such as DNA and RNA (Egholm et al., 1993; PCT/EP/01219). A peptide nucleic acid generally comprises one or more nucleotides or nucleosides that comprise a nucleobase moiety, a nucleobase linker moeity that is not a 5-carbon sugar, and/or a backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

In certain embodiments, a nucleic acid analogue such as a peptide nucleic acid may be used to inhibit nucleic acid amplification, such as in PCR, to reduce false positives and discriminate between single base mutants, as described in U.S. Pat. No. 5,891,625. Other modifications and uses of nucleic acid analogs are known in the art, and are encompassed herein. In a non-limiting example, U.S. Pat. No. 5,786,461 describes PNAs with amino acid side chains attached to the PNA backbone to enhance solubility of the molecule. In another example, the cellular uptake property of PNAs is increased by attachment of a lipophilic group. U.S. application Ser. No. 117,363 describes several alkylamino moeities used to enhance cellular uptake of a PNA. Another example is described in U.S. Pat. Nos. 5,766,855, 5,719,262, 5,714,331 and 5,736,336, which describe PNAs comprising naturally and non-naturally occurring nucleobases and alkylamine side chains that provide improvements in sequence specificity, solubility and/or binding affinity relative to a naturally occurring nucleic acid.

### F. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosponate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 1989, incorporated herein by reference).

### G. Purification of Nucleic Acids

A nucleic acid may be purified on polyacrylamide gels, cesium chloride centrifugation gradients, or by any other means known to one of ordinary skill in the art (see for example, Sambrook et al., 1989, incorporated herein by reference).

In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

### H. Nucleic Acid Segments

In certain embodiments, the nucleic acid is a nucleic acid segment. As used herein, the term “nucleic acid segment,” are smaller fragments of a nucleic acid, such as for non-limiting example, those that encode only part of the peptide or polypeptide sequence. Thus, a “nucleic acid segment” may comprise any part of a gene sequence, of from about 2 nucleotides to the full length of the peptide or polypeptide encoding region.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

### I. Nucleic Acid Complements

The present invention also encompasses a nucleic acid that is complementary to an A20 nucleic acid. In particular embodiments the invention encompasses a nucleic acid or a nucleic acid segment complementary to the sequence set forth in SEQ ID NO: 1 or SEQ ID NO: 3. A nucleic acid is “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule.

As used herein, the term “complementary” or “complement(s)” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

### J. Hybridization

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

As used herein “wild-type” refers to the naturally occurring sequence of a nucleic acid at a genetic locus in the genome of an organism, or a sequence transcribed or translated from such a nucleic acid. Thus, the term “wild-type” also may refer to an amino acid sequence encoded by a nucleic acid. As a genetic locus may have more than one sequence or alleles in a population of individuals, the term “wild-type” encompasses all such naturally occurring allele(s). As used herein the term “polymorphic” means that variation exists (i.e., two or more alleles exist) at a genetic locus in the individuals of a population. As used herein “mutant” refers to a change in the sequence of a nucleic acid or its encoded protein, polypeptide or peptide that is the result of the hand of man.

The present invention also concerns the isolation or creation of a recombinant construct or a recombinant host cell through the application of recombinant nucleic acid technology known to those of skill in the art or as described herein. A recombinant construct or host cell may comprise an A20 nucleic acid, and may express an A20 protein, peptide or peptide, or at least one biologically functional equivalent thereof.

Herein certain embodiments, a “gene” refers to a nucleic acid that is transcribed. In certain aspects, the gene includes regulatory sequences involved in transcription, or message production or composition. In particular embodiments, the gene comprises transcribed sequences that encode for a protein, polypeptide or peptide. As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like.

“Isolated substantially away from other coding sequences” means that the gene of interest forms the significant part of the coding region of the nucleic acid, or that the nucleic acid does not contain large portions of naturally-occurring coding nucleic acids, such as large chromosomal fragments, other functional genes, RNA or cDNA coding regions. Of course, this refers to the nucleic acid as originally isolated, and does not exclude genes or coding regions later added to the nucleic acid by the hand of man.

The nucleic acid(s) of the present invention, regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). As used herein, a “nucleic acid construct” is a nucleic acid engineered or altered by the hand of man, and generally comprises one or more nucleic acid sequences organized by the hand of man.

In a non-limiting example, one or more nucleic acid constructs may be prepared that include a contiguous stretch of nucleotides such as those identical to or complementary to SEQ ID NO: 1 or SEQ ID NO: 3. A nucleic acid construct may be about 3, about 5, about 8, about 10 to about 14, or about 15, about 20, about 30, about 40, about 50, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges”, as used herein, means any length or range including or between the quoted values (i.e., all integers including and between such values). Non-limiting examples of intermediate lengths include about 11, about 12, about 13, about 16, about 17, about 18, about 19, etc.; about 21, about 22, about 23, etc.; about 31, about 32, etc.; about 51, about 52, about 53, etc.; about 101, about 102, about 103, etc.; about 151, about 152, about 153, etc.; about 1,001, about 1002, etc,; about 50,001, about 50,002, etc; about 750,001, about 750,002, etc.; about 1,000,001, about 1,000,002, etc. Non-limiting examples of intermediate ranges include about 3 to about 32, about 150 to about 500,001, about 3,032 to about 7,145, about 5,000 to about 15,000, about 20,007 to about 1,000,003, etc.

In particular embodiments, the invention concerns one or more recombinant vector(s) comprising nucleic acid sequences that encode an A20 protein, polypeptide or peptide that includes within its amino acid sequence a contiguous amino acid sequence in accordance with, or essentially as set forth in, SEQ ID NO: 2, corresponding to human A20. In other embodiments, the invention concerns recombinant vector(s) comprising nucleic acid sequences that encode a mouse A20 protein, polypeptide or peptide that includes within its amino acid sequence a contiguous amino acid sequence in accordance with, or essentially as set forth in SEQ ID NO: 4. In particular aspects, the recombinant vectors are DNA vectors.

The term “a sequence essentially as set forth in SEQ ID NO: 2” or “a sequence essentially as set forth in SEQ ID NO: 2” means that the sequence substantially corresponds to a portion of SEQ ID NO: 2 and has relatively few amino acids that are not identical to, or a biologically functional equivalent of, the amino acids of SEQ ID NO: 2. Thus, “a sequence essentially as set forth in SEQ ID NO: 1” or “a sequence essentially as set forth in SEQ ID NO: 1 ” encompasses nucleic acids, nucleic acid segments, and genes that comprise part or all of the nucleic acid sequences as set forth in SEQ ID NO: 1.

The term “biologically functional equivalent” is well understood in the art and is further defined in detail herein. Accordingly, a sequence that has between about 70% and about 80%; or more preferably, between about 81% and about 90%; or even more preferably, between about 91% and about 99% of amino acids that are identical or functionally equivalent to the amino acids as set forth as the sequence of an A20 protein, provided the biological activity of the protein, polypeptide or peptide is maintained.

In certain other embodiments, the invention concerns at least one recombinant vector that include within its sequence a nucleic acid sequence essentially as set forth in SEQ ID NO: 1 or SEQ ID NO: 3. In particular embodiments, the recombinant vector comprises DNA sequences that encode protein(s), polypeptide(s) or peptide(s) exhibiting the ability to inhibit TNF function.

The term “functionally equivalent codon” is used herein to refer to codons that encode the same amino acid, such as the six codons for arginine and serine, and also refers to codons that encode biologically equivalent amino acids. For optimization of expression of A20 in human cells, the codons are shown in Table 2 in preference of use from left to right. Thus, the most preferred codon for alanine is thus “GCC”, and the least is “GCG” (see Table 2, below). Codon usage for various organisms and organelles can be found at the website http://www.kazusa.or.jp/codon/, incorporated herein by reference, allowing one of skill in the art to optimize codon usage for expression in various organisms using the disclosures herein. Thus, it is contemplated that codon usage may be optimized for other animals, as well as other organisms such as a prokaryote (e.g., an eubacteria, an archaea), an eukaryote (e.g., a protist, a plant, a fungi, an animal), a virus and the like, as well as organelles that contain nucleic acids, such as mitochondria, chloroplasts and the like, based on the preferred codon usage as would be known to those of ordinary skill in the art.

TABLE 2

Preferred Human DNA Codons

| | |
|---|---|
| Amino Acids | Codons |

| | | | |
|---|---|---|---|
| Alanine | Ala | A | GCC GCT GCA GCG |
| Cysteine | Cys | C | TGC TGT |
| Aspartic acid | Asp | D | GAC GAT |
| Glutamic acid | Glu | B | GAG GAA |
| Phenylalanine | Phe | F | TTC TTT |
| Glycine | Gly | G | GGC GGG GGA GGT |
| Histidine | His | H | CAC CAT |
| Isoleucine | Ile | I | ATC ATT ATA |
| Lysine | Lys | K | AAG AAA |
| Leucine | Leu | L | CTG CTC TTG CTT CTA TTA |
| Methionine | Met | M | ATG |
| Asparagine | Asn | N | AAC AAT |
| Proline | Pro | P | CCC CCT CCA CCG |
| Glutamine | Gln | Q | CAG CAA |
| Arginine | Mg | R | CGC AGG CGG AGA CGA CGT |
| Serine | Ser | S | AGC TCC TCT AGT TCA TCG |
| Threonine | Thr | T | ACC ACA ACT ACG |
| Valine | Val | V | GTG GTC GTT GTA |
| Tryptophan | Trp | W | TGG |
| Tyrosine | Tyr | Y | TAC TAT |

It will also be understood that amino acid sequences or nucleic acid sequences may include additional residues, such as additional N- or C-terminal amino acids or 5′ or 3′ sequences, or various combinations thereof, and yet still be essentially as set forth in one of the sequences disclosed herein, so long as the sequence meets the criteria set forth above, including the maintenance of biological protein, polypeptide or peptide activity where expression of a proteinaceous composition is concerned. The addition of terminal sequences particularly applies to nucleic acid sequences that may, for example, include various non-coding sequences flanking either of the 5′ and/or 3′ portions of the coding region or may include various internal sequences, i.e., introns, which are known to occur within genes.

It will also be understood that this invention is not limited to the particular nucleic acid or amino acid sequences of SEQ ID NOS: 1-4. Recombinant vectors and isolated nucleic acid segments may therefore variously include these coding regions themselves, coding regions bearing selected alterations or modifications in the basic coding region, and they may encode larger polypeptides or peptides that nevertheless include such coding regions or may encode biologically functional equivalent proteins, polypeptide or peptides that have variant amino acids sequences.

The nucleic acids of the present invention encompass biologically functional equivalent A20 proteins, polypeptides, or peptides. Such sequences may arise as a consequence of codon redundancy or functional equivalency that are known to occur naturally within nucleic acid sequences or the proteins, polypeptides or peptides thus encoded. Alternatively, functionally equivalent proteins, polypeptides or peptides may be created via the application of recombinant DNA technology, in which changes in the protein, polypeptide or peptide structure may be engineered, based on considerations of the properties of the amino acids being exchanged. Changes designed by man may be introduced, for example, through the application of site-directed mutagenesis techniques as discussed herein below, e.g., to introduce improvements or alterations to the antigenicity of the protein, polypeptide or peptide, or to test mutants in order to examine A20 protein, polypeptide or peptide activity at the molecular level.

Fusion proteins, polypeptides or peptides may be prepared, e.g., where the A20 coding regions are aligned within the same expression unit with other proteins, polypeptides or peptides having desired functions. Non-limiting examples of such desired functions of expression sequences include purification or immunodetection purposes for the added expression sequences, e.g., proteinaceous compositions that may be purified by affinity chromatography or the enzyme labeling of coding regions, respectively.

Encompassed by the invention are nucleic acid sequences encoding relatively small peptides or fusion peptides, such as, for example, peptides of from about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, to about 100 amino acids in length, or more preferably, of from about 15 to about 30 amino acids in length; as set forth in SEQ ID NO: 2 or SEQ ID NO: 4 and also larger polypeptides up to and including proteins corresponding to the full-length sequences set forth in SEQ ID NO: 2 and/or SEQ ID NO: 4 or homologs thereof.

As used herein an “organism” may be a prokaryote, eukaryote, virus and the like. As used herein the term “sequence” encompasses both the terms “nucleic acid” and “proteancecous” or “proteanaceous composition.” As used herein, the term “proteinaceous composition” encompasses the terms “protein”, “polypeptide” and “peptide.” As used herein “artificial sequence” refers to a sequence of a nucleic acid not derived from sequence naturally occurring at a genetic locus, as well as the sequence of any proteins, polypeptides or peptides encoded by such a nucleic acid. A “synthetic sequence”, refers to a nucleic acid or proteinaceous composition produced by chemical synthesis in vitro, rather than enzymatic production in vitro (i.e., an “enzymatically produced” sequence) or biological production in vivo (i.e., a “biologically produced” sequence).

### IV. Proteinaceous Compositions

In certain embodiments, the present invention concerns novel compositions comprising at least one proteinaceous molecule. The proteinaceous molecule may be used as a candidate substance to be screened as a modulator of A20 function. The proteinaceous molecule may also be used, for example, in a pharmaceutical composition for the delivery of a therapeutic agent or as part of a screening assay in the determination of A20 polypeptide activity. As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments the size of the at least one proteinaceous molecule may comprise, but is not limited to, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino molecule residues, and any range derivable therein.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid, including but not limited to those shown on Table 3 below.

TABLE 3
Modified and Unusual Amino Acids

| Abbr. | Amino Acid |
|---|---|
| Aad | 2-Aminoadipic acid |
| Baad | 3-Aminoadipic acid |
| Bala | β-alanine, β-Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| Baib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| Ahyl | allo-Hydroxylysine |
| 3Hyp | 3-Hydroxyproline |
| 4Hyp | 4-Hydroxyproline |
| Ide | Isodesmosine |
| AIle | allo-Isoleucine |
| MeGly | N-Methylglycine, |
| | sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Organisms include, but are not limited to, Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments, the proteinaceous composition may comprise at least one antibody. It is contemplated that antibodies to specific tissues may bind the tissue(s) and foster tighter adhesion of the glue to the tissues after welding. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

Proteins and peptides suitable for use in this invention may be autologous proteins or peptides, although the invention is clearly not limited to the use of such autologous proteins. As used herein, the term “autologous protein, polypeptide or peptide” refers to a protein, polypeptide or peptide which is derived or obtained from an organism. Organisms that may be used include, but are not limited to, a bovine, a reptilian, an amphibian, a piscine, a rodent, an avian, a canine, a feline, a fungal, a plant, or a prokaryotic organism, with a selected animal or human subject being preferred. The “autologous protein, polypeptide or peptide” may then be used as a component of a composition intended for application to the selected animal or human subject. In certain aspects, the autologous proteins or peptides are prepared, for example from whole plasma of the selected donor. The plasma is placed in tubes and placed in a freezer at about −80° C. for at least about 12 hours and then centrifuged at about 12,000 times g for about 15 minutes to obtain the precipitate. The precipitate, such as fibrinogen may be stored for up to about one year (Oz, 1990).

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

To prepare a composition comprising the candidate substance or A20 polypeptide, it may be desirable to purify the components or variants thereof According to one embodiment of the present invention, purification of a peptide comprising the candidate substance or A20 polypeptide can be utilized ultimately to operatively link this domain with a selective agent. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide, such as an A20 polypeptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition, such as the A20 polypeptide, that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (e.g., alter pH, ionic strength, and temperature.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand also should provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

The present invention also describes an A20 polypeptide, including an fusion protein, for use in various embodiments of the present invention. The peptides of the invention can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Peptides with at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 50, 55, 60, 65, 70, 75, 80, 85, 90, 95 or up to about 100 amino acid residues are contemplated by the present invention.

The compositions of the invention may include a peptide comprising an A20 polypeptide that has been modified to enhance its activity or to render it biologically protected. Biologically protected peptides have certain advantages over unprotected peptides when administered to human subjects and, as disclosed in U.S. Pat. No. 5,028,592, incorporated herein by reference, protected peptides often exhibit increased pharmacological activity.

Compositions for use in the present invention may also comprise peptides that include all L-amino acids, all D-amino acids, or a mixture thereof The use of D-amino acids may confer additional resistance to proteases naturally found within the human body and are less immunogenic and can therefore be expected to have longer biological half lives.

### V. Mutagenesis

Where employed, mutagenesis will be accomplished by a variety of standard, mutagenic procedures. Mutation is the process whereby changes occur in the quantity or structure of an organism. Mutation can involve modification of the nucleotide sequence of a single gene, blocks of genes or whole chromosome. Changes in single genes may be the consequence of point mutations which involve the removal, addition or substitution of a single nucleotide base within a DNA sequence, or they may be the consequence of changes involving the insertion or deletion of large numbers of nucleotides.

Mutations can arise spontaneously as a result of events such as errors in the fidelity of DNA replication or the movement of transposable genetic elements (transposons) within the genome. They also are induced following exposure to chemical or physical mutagens. Such mutation-inducing agents include ionizing radiations, ultraviolet light and a diverse array of chemical such as alkylating agents and polycyclic aromatic hydrocarbons all of which are capable of interacting either directly or indirectly (generally following some metabolic biotransformations) with nucleic acids. The DNA lesions induced by such environmental agents may lead to modifications of base sequence when the affected DNA is replicated or repaired and thus to a mutation. Mutation also can be site-directed through the use of particular targeting methods.

### A. Random Mutagenesis

### 1) Insertional Mutagenesis

Insertional mutagenesis is based on the inactivation of a gene via insertion of a known DNA fragment. Because it involves the insertion of some type of DNA fragment, the mutations generated are generally loss-of-function, rather than gain-of-function mutations. However, there are several examples of insertions generating gain-of-function mutations (Oppenheimer et al. 1991). Insertion mutagenesis has been very successful in bacteria and Drosophila (Cooley et al. 1988) and recently has become a powerful tool in corn (Schmidt et al. 1987); Arabidopsis; (Marks et al., 1991; Koncz et al. 1990); and Antirrhinum (Sommer et al. 1990).

Transposable genetic elements are DNA sequences that can move (transpose) from one place to another in the genome of a cell. The first transposable elements to be recognized were the Activator/Dissociation elements of Zea mays (NcClintock, 1957). Since then, they have been identified in a wide range of organisms, both prokaryotic and eukaryotic.

Transposable elements in the genome are characterized by being flanked by direct repeats of a short sequence of DNA that has been duplicated during transposition and is called a target site duplication. Virtually all transposable elements whatever their type, and mechanism of transposition, make such duplications at the site of their insertion. In some cases the number of bases duplicated is constant, in other cases it may vary with each transposition event. Most transposable elements have inverted repeat sequences at their termini, these terminal inverted repeats may be anything from a few bases to a few hundred bases long and in many cases they are known to be necessary for transposition.

Prokaryotic transposable elements have been most studied in E. coli and Gram negative bacteria, but also are present in Gram positive bacteria. They are generally termed insertion sequences if they are less than about 2 kB long, or transposons if they are longer. Bacteriophages such as mu and D108, which replicate by transposition, make up a third type of transposable element. elements of each type encode at least one polypeptide a transposase, required for their own transposition. Transposons often further include genes coding for function unrelated to transposition, for example, antibiotic resistance genes.

Transposons can be divided into two classes according to their structure. First, compound or composite transposons have copies of an insertion sequence element at each end, usually in an inverted orientation. These transposons require transposases encoded by one of their terminal IS elements. The second class of transposon have terminal repeats of about 30 base pairs and do not contain sequences from IS elements.

Transposition usually is either conservative or replicative, although in some cases it can be both. In replicative transposition, one copy of the transposing element remains at the donor site, and another is inserted at the target site. In conservative transposition, the transposing element is excised from one site and inserted at another.

Eukaryotic elements also can be classified according to their structure and mechanism of transportation. The primary distinction is between elements that transpose via an RNA intermediate, and elements that transpose directly from DNA to DNA.

Elements that transpose via an RNA intermediate often are referred to as retrotransposons, and their most characteristic feature is that they encode polypeptides that are believed to have reverse transcriptionase activity. There are two types of retrotransposon. Some resemble the integrated proviral DNA of a retrovirus in that they have long direct repeat sequences, long terminal repeats (LTRs), at each end. The similarity between these retrotransposons and proviruses extends to their coding capacity. They contain sequences related to the gag and pol genes of a retrovirus, suggesting that they transpose by a mechanism related to a retroviral life cycle. Retrotransposons of the second type have no terminal repeats. They also code for gag- and pol-like polypeptides and transpose by reverse transcription of RNA intermediates, but do so by a mechanism that differs from that or retrovirus-like elements. Transposition by reverse transcription is a replicative process and does not require excision of an element from a donor site.

Transposable elements are an important source of spontaneous mutations, and have influenced the ways in which genes and genomes have evolved. They can inactivate genes by inserting within them, and can cause gross chromosomal rearrangements either directly, through the activity of their transposases, or indirectly, as a result of recombination between copies of an element scattered around the genome. Transposable elements that excise often do so imprecisely and may produce alleles coding for altered gene products if the number of bases added or deleted is a multiple of three.

Transposable elements themselves may evolve in unusual ways. If they were inherited like other DNA sequences, then copies of an element in one species would be more like copies in closely related species than copies in more distant species. This is not always the case, suggesting that transposable elements are occasionally transmitted horizontally from one species to another.

### 2) Chemical Mutagenesis

Chemical mutagenesis offers certain advantages, such as the ability to find a fill range of mutant alleles with degrees of phenotypic severity, and is facile and inexpensive to perform. The majority of chemical carcinogens produce mutations in DNA. Benzo[a]pyrene, N-acetoxy-2-acetyl aminofluorene and aflotoxin B1 cause GC to TA transversions in bacteria and mammalian cells. Benzo[a]pyrene also can produce base substitutions such as AT to TA. N-nitroso compounds produce GC to AT transitions. Alkylation of the 04 position of thymine induced by exposure to n-nitrosoureas results in TA to CG transitions.

A high correlation between mutagenicity and carcinogenity is the underlying assumption behind the Ames test (McCann et al., 1975) which speedily assays for mutants in a bacterial system, together with an added rat liver homogenate, which contains the microsomal cytochrome P450, to provide the metabolic activation of the mutagens where needed.

In vertebrates, several carcinogens have been found to produce mutation in the ras proto-oncogene. N-nitroso-N-methyl urea induces mammary, prostate and other carcinomas in rats with the majority of the tumors showing a G to A transition at the second position in codon 12 of the Ha-ras oncogene. Benzo[a]pyrene-induced skin tumors contain A to T transformation in the second codon of the Ha-ras gene.

### 3) Radiation Mutagenesis

The integrity of biological molecules is degraded by the ionizing radiation. Adsorption of the incident energy leads to the formation of ions and free radicals, and breakage of some covalent bonds. Susceptibility to radiation damage appears quite variable between molecules, and between different crystalline forms of the same molecule. It depends on the total accumulated dose, and also on the dose rate (as once free radicals are present, the molecular damage they cause depends on their natural diffusion rate and thus upon real time). Damage is reduced and controlled by making the sample as cold as possible.

Ionizing radiation causes DNA damage and cell killing, generally proportional to the dose rate. Ionizing radiation has been postulated to induce multiple biological effects by direct interaction with DNA, or through the formation of free radical species leading to DNA damage (Hall, 1988). These effects include gene mutations, malignant transformation, and cell killing. Although ionizing radiation has been demonstrated to induce expression of certain DNA repair genes in some prokaryotic and lower eukaryotic cells, little is known about the effects of ionizing radiation on the regulation of mammalian gene expression (Borek, 1985). Several studies have described changes in the pattern of protein synthesis observed after irradiation of mammalian cells. For example, ionizing radiation treatment of human malignant melanoma cells is associated with induction of several unidentified proteins (Boothman et al., 1989). Synthesis of cyclin and co-regulated polypeptides is suppressed by ionizing radiation in rat REF52 cells, but not in oncogene-transformed REF52 cell lines (Lambert and Borek, 1988). Other studies have demonstrated that certain growth factors or cytokines may be involved in x-ray-induced DNA damage. In this regard, platelet-derived growth factor is released from endothelial cells after irradiation (Witte, et al., 1989).

In the present invention, the term “ionizing radiation” means radiation comprising particles or photons that have sufficient energy or can produce sufficient energy via nuclear interactions to produce ionization (gain or loss of electrons). An exemplary and preferred ionizing radiation is an x-radiation. The amount of ionizing radiation needed in a given cell generally depends upon the nature of that cell. Typically, an effective expression-inducing dose is less than a dose of ionizing radiation that causes cell damage or death directly. Means for determining an effective amount of radiation are well known in the art.

In a certain embodiments, an effective expression inducing amount is from about 2 to about 30 Gray (Gy) administered at a rate of from about 0.5 to about 2 Gy/minute. Even more preferably, an effective expression inducing amount of ionizing radiation is from about 5 to about 15 Gy. In other embodiments, doses of 2-9 Gy are used in single doses. An effective dose of ionizing radiation may be from 10 to 100 Gy, with 15 to 75 Gy being preferred, and 20 to 50 Gy being more preferred.

Any suitable means for delivering radiation to a tissue may be employed in the present invention in addition to external means. For example, radiation may be delivered by first providing a radiolabeled antibody that immunoreacts with an antigen of the tumor, followed by delivering an effective amount of the radiolabeled antibody to the tumor. In addition, radioisotopes may be used to deliver ionizing radiation to a tissue or cell.

### 4) In Vitro Scanning Mutagenesis

Random mutagenesis also may be introduced using error prone PCR (Cadwell and Joyce, 1992). The rate of mutagenesis may be increased by performing PCR in multiple tubes with dilutions of templates.

One particularly useful mutagenesis technique is alanine scanning mutagenesis in which a number of residues are substituted individually with the amino acid alanine so that the effects of losing side-chain interactions can be determined, while minimizing the risk of large-scale perturbations in protein conformation (Cunningham et al., 1989).

In recent years, techniques for estimating the equilibrium constant for ligand binding using minuscule amounts of protein have been developed (Blackburn et al., 1991; U.S. Pat. Nos. 5,221,605 and 5,238,808). The ability to perform functional assays with small amounts of material can be exploited to develop highly efficient, in vitro methodologies for the saturation mutagenesis of antibodies. The inventors bypassed cloning steps by combining PCR mutagenesis with coupled in vitro transcription/translation for the high throughput generation of protein mutants. Here, the PCR products are used directly as the template for the in vitro transcription/translation of the mutant single chain antibodies. Because of the high efficiency with which all 19 amino acid substitutions can be generated and analyzed in this way, it is now possible to perform saturation mutagenesis on numerous residues of interest, a process that can be described as in vitro scanning saturation mutagenesis (Burks et al., 1997).

In vitro scanning saturation mutagenesis provides a rapid method for obtaining a large amount of structure-function information including: (i) identification of residues that modulate ligand binding specificity, (ii) a better understanding of ligand binding based on the identification of those amino acids that retain activity and those that abolish activity at a given location, (iii) an evaluation of the overall plasticity of an active site or protein subdomain, (iv) identification of amino acid substitutions that result in increased binding.

### 5) Random Mutagenesis by Fragmentation and Reassmbly

A method for generating libraries of displayed polypeptides is described in U.S. Pat. No. 5,380,721. The method comprises obtaining polynucleotide library members, pooling and fragmenting the polynucleotides, and reforming fragments therefrom, performing PCR amplification, thereby homologously recombining the fragments to form a shuffled pool of recombined polynucleotides.

### B. Site-Directed Mutagenesis

Structure-guided site-specific mutagenesis represents a powerful tool for the dissection and engineering of protein-ligand interactions (Wells, 1996, Braisted et al., 1996). The technique provides for the preparation and testing of sequence variants by introducing one or more nucleotide sequence changes into a selected DNA.

Site-specific mutagenesis uses specific oligonucleotide sequences which encode the DNA sequence of the desired mutation, as well as a sufficient number of adjacent, unmodified nucleotides. In this way, a primer sequence is provided with sufficient size and complexity to form a stable duplex on both sides of the deletion junction being traversed. A primer of about 17 to 25 nucleotides in length is preferred, with about 5 to 10 residues on both sides of the junction of the sequence being altered.

The technique typically employs a bacteriophage vector that exists in both a single-stranded and double-stranded form. Vectors useful in site-directed mutagenesis include vectors such as the M13 phage. These phage vectors are commercially available and their use is generally well known to those skilled in the art. Double-stranded plasmids are also routinely employed in site-directed mutagenesis, which eliminates the step of transferring the gene of interest from a phage to a plasmid.

In general, one first obtains a single-stranded vector, or melts two strands of a double-stranded vector, which includes within its sequence a DNA sequence encoding the desired protein or genetic element. An oligonucleotide primer bearing the desired mutated sequence, synthetically prepared, is then annealed with the single-stranded DNA preparation, taking into account the degree of mismatch when selecting hybridization conditions. The hybridized product is subjected to DNA polymerizing enzymes such as E. coli polymerase I (Klenow fragment) in order to complete the synthesis of the mutation-bearing strand. Thus, a heteroduplex is formed, wherein one strand encodes the original non-mutated sequence, and the second strand bears the desired mutation. This heteroduplex vector is then used to transform appropriate host cells, such as E. coli cells, and clones are selected that include recombinant vectors bearing the mutated sequence arrangement.

Comprehensive information on the functional significance and information content of a given residue of protein can best be obtained by saturation mutagenesis in which all 19 amino acid substitutions are examined. The shortcoming of this approach is that the logistics of multiresidue saturation mutagenesis are daunting (Warren et al., 1996, Brown et al., 1996; Zeng et at., 1996; Burton and Barbas, 1994; Yelton et al., 1995; Jackson et al., 1995; Short et at, 1995; Wong et al., 1996; Hilton et al., 1996). Hundreds, and possibly even thousands, of site specific mutants must be studied. However, improved techniques make production and rapid screening of mutants much more straightforward. See also, U.S. Pat. Nos. 5,798,208 and 5,830,650, for a description of “walk-through” mutagenesis.

Other methods of site-directed mutagenesis are disclosed in U.S. Pat. Nos. 5,220,007; 5,284,760; 5,354,670; 5,366,878; 5,389,514; 5,635,377; and 5,789,166.

### VI. Pharmaceutical Preparations

Pharmaceutical compositions of the present invention comprise an effective amount of one or more active compound and optionally an additional agent dissolved or dispersed in a pharmaceutical composition. The phrases “pharmaceutical composition” refers to molecular entities and compositions that do not produce an adverse, allergic or other untoward reaction when administered to an animal, such as, for example, a human, as appropriate. The preparation of an pharmaceutical composition that contains at least one active compound or a candidate substance and optionally an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutical composition” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The active compound may comprise different types of carriers depending on whether it is to be administered in solid, liquid or aerosol form, and whether it need to be sterile for such routes of administration as injection. The active compound can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g.. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference). Administration by injection is preferred.

The actual dosage amount of a composition of the active compound administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof

The active compound or candidate substance may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments, the active compound is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof

As used herein the specification, “a” or “an” may mean one or more. As used herein in the claim(s), when used in conjunction with the word “comprising”, the words “a” or “an” may mean one or more than one. As used herein “another” may mean at least a second or more.

### VI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### A20 is a Novel Molecule Which Regulates TNF Responses

To further evaluate the role of macrophage derived cytokines in stimulating intestinal immune responses, the role of the pro-inflammatory cytokine TNF was investigated. TNF is released predominantly from myeloid cells such as macrophages in response to bacterial cell wall polysaccharides such as lipopolysaccharide (LPS), abundant in the microbe-rich intestinal milieu. As described below, dysregulated TNF expression causes intestinal inflammation (Kontoyiannis et al., 1999). It was hypothesized that cellular responses to TNF should also be properly regulated to maintain mucosal immune homeostasis.

As various lines of evidence suggested that TNF induced signals regulate intestinal inflammation, it was sought to understand the molecular mechanisms by which these signals are regulated. TNF signals, largely mediated through TNFR1, lead to the activation NF-κB and JNK pathways, as well as caspase mediated PCD pathways (Chan et al., 2000). NF-κB activation leads to the transcription of multiple pro-inflammatory as well as anti-apoptotic genes in diverse cell types (Schmid and Adler, 2000). In searching for molecules that might regulate cellular responses to TNF, A20, a TNF induced molecule thought to regulate both TNF induced NF-κB signaling and TNF induced PCD responses in cell lines, was identified (Opipari et al., 1990; Tewari et al, 1995). To determine the in vivo functions of A20, the inventors targeted the A20 gene by homologous recombination in embryonic stem (ES) cells in order to create A20 deficient (A20^{-/-}) mice (Lee et al., 2000).

To examine how TNF responses are normally held in check, A20 was tested. A20 is a molecule thought to be selectively expressed in lymphocytes and to regulate NF-κB responses to TNF (Opipari et al., 1990; Tewari et al., 1995). As described is below, it was found that A20 is a critical regulator of TNF induced NF-κB activation in virtually all tissues (not only lymphoid tissues), and that the failure to regulate NF-κB activity in A20 deficient mice leads to profound intestinal inflammation. Thus, A20 is critical for terminating TNF signals and restricting intestinal inflammation in vivo.

Therefore, it was shown that TNF induced responses are regulated by a novel protein called A20. A20 terminates TNF induced NF-κB signals and protects cells from TNF induced PCD. The failure of A20 to terminate TNF induced responses leads to profound intestinal inflammation and damage, demonstrating A20's critical role in regulating mucosal immune responses. These exciting findings provide unique opportunities to interrogate the mechanisms by which TNF mediated inflammatory responses are regulated in vivo.

### Example 2

### Generation and Histology of A20^{-/-} mice

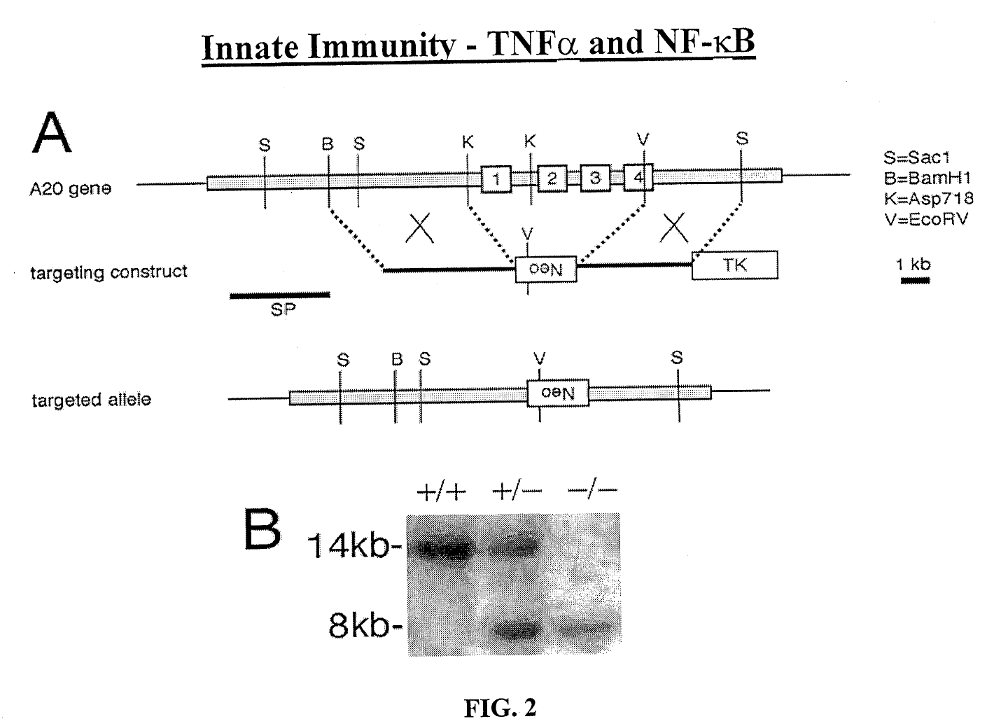

To disrupt the A20 gene in mice, the inventors cloned and mapped a 14 kb genomic clone encompassing the murine A20 gene. A gene targeting construct designed to eliminate the ATG start codon and the first 738 base pairs of the coding sequence (corresponding to residues 1-246) was transfected into ES cells (FIG. 2). Correctly targeted ES clones were identified by Southern blotting (FIG. 2) and injected into C57B1/6 blastocysts, after which male chimeric mice were bred to C57B1/6 females to obtain germline transmission of the A20 mutant allele.

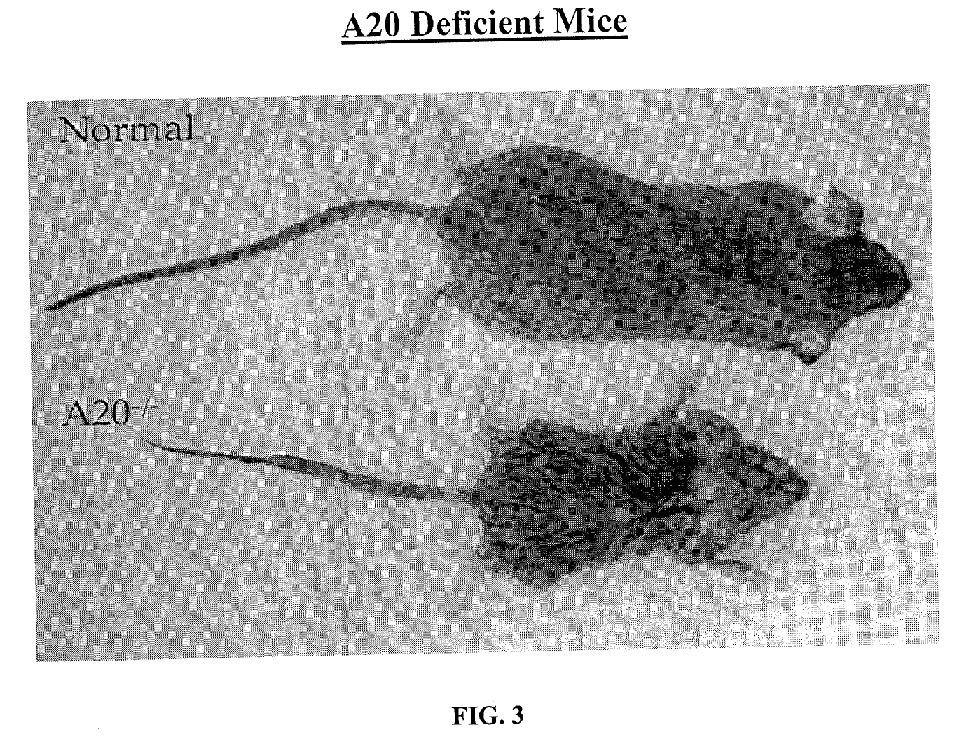

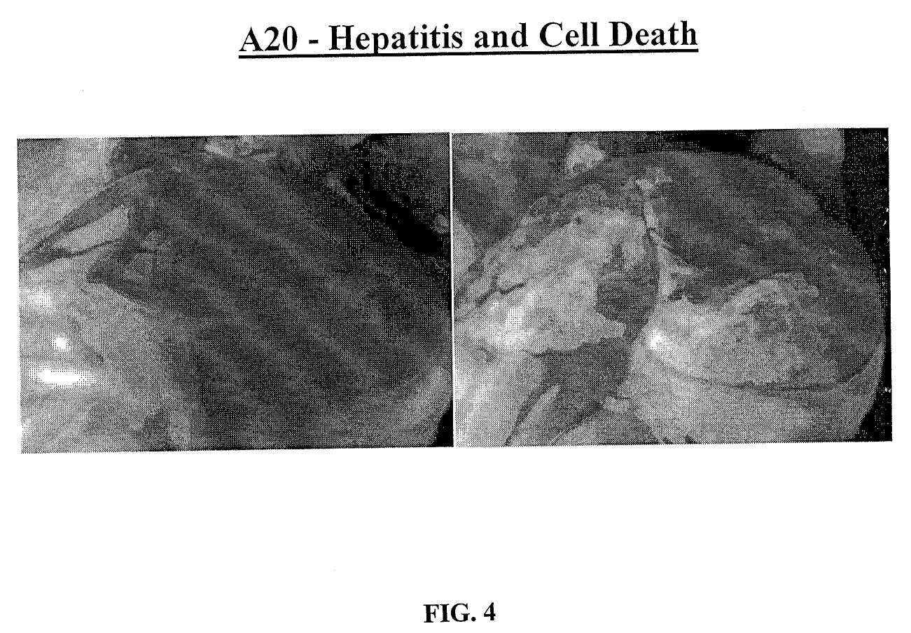

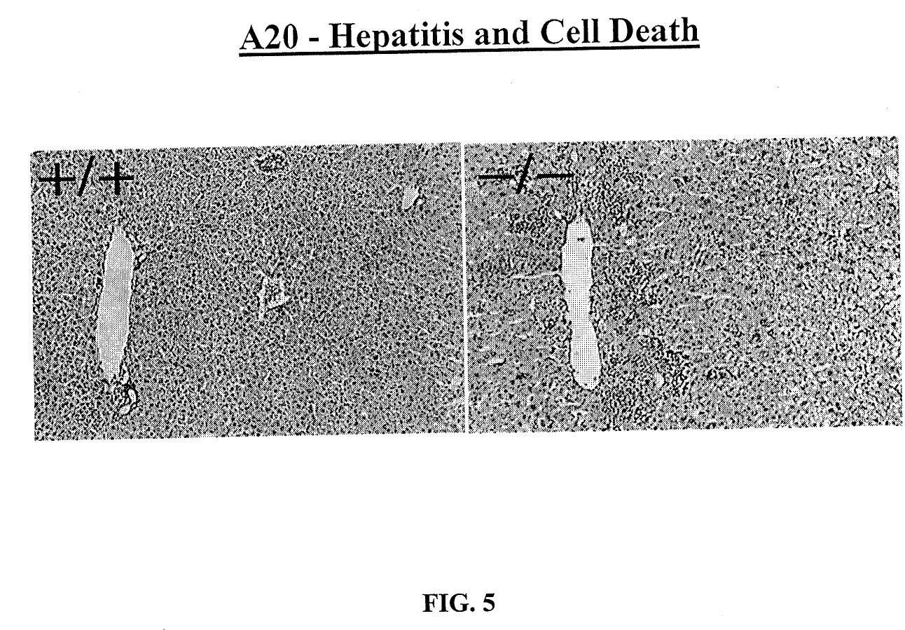

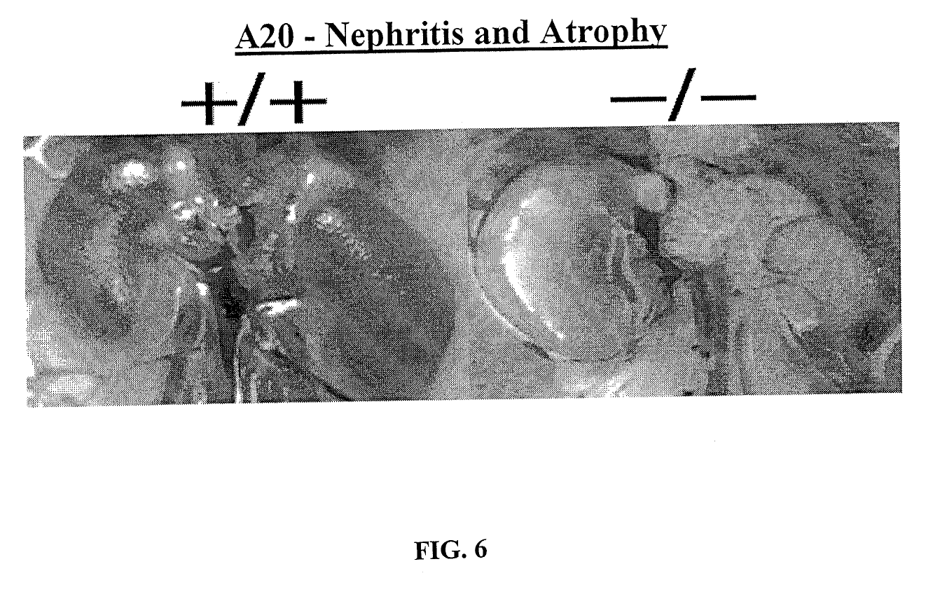

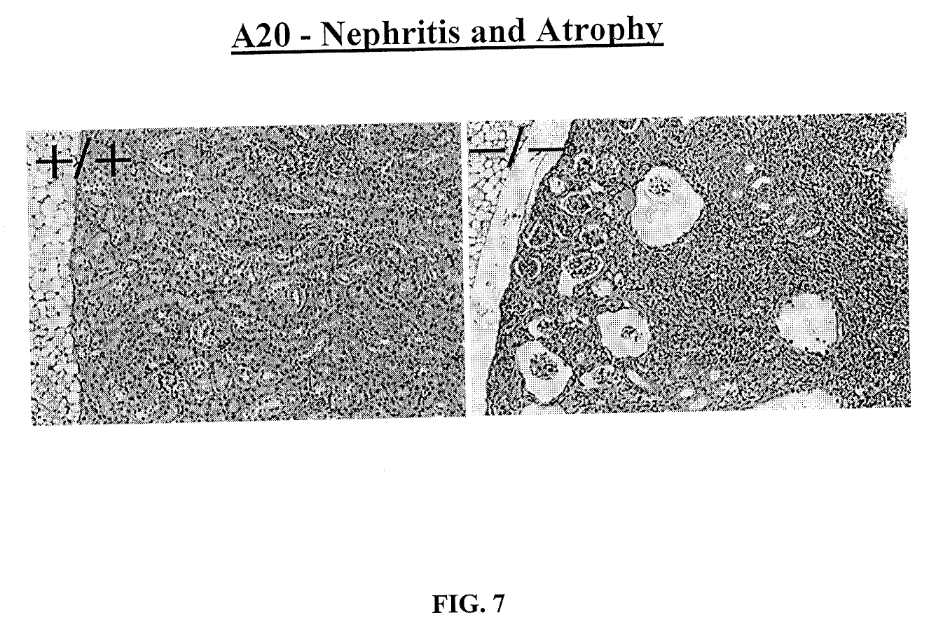

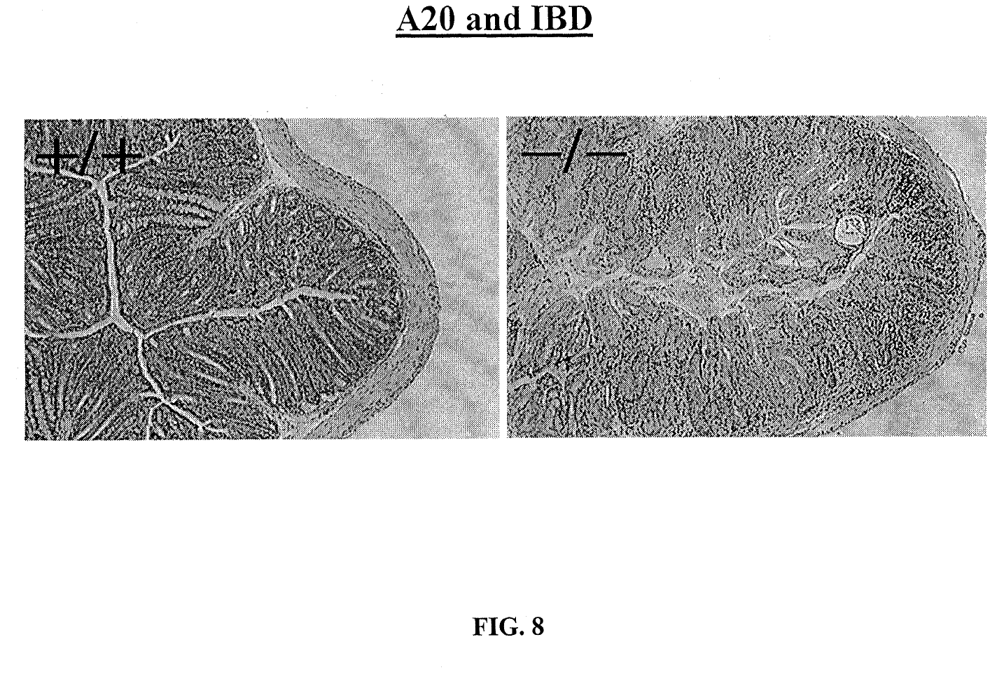

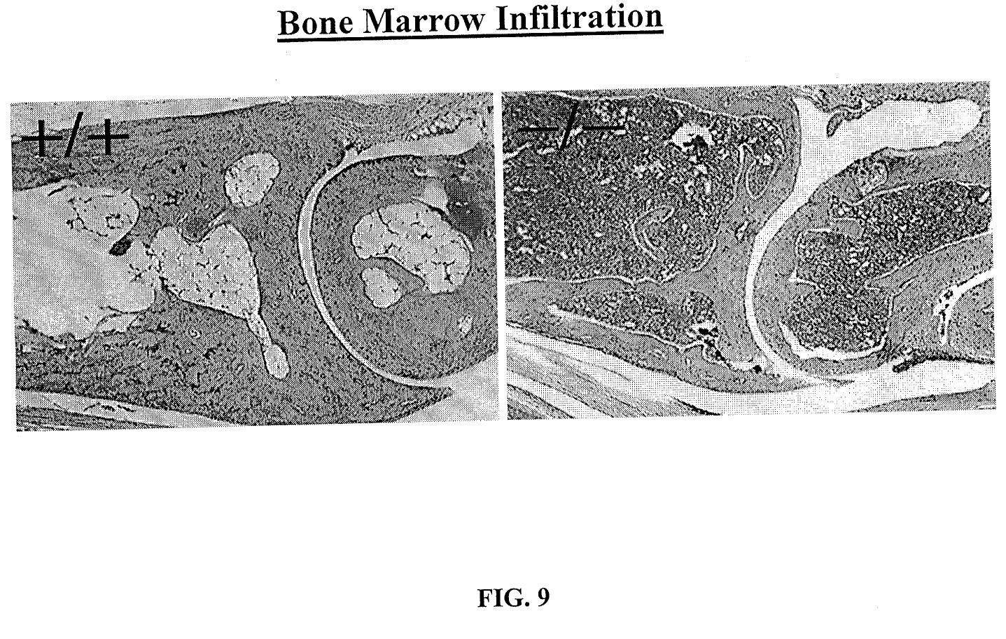

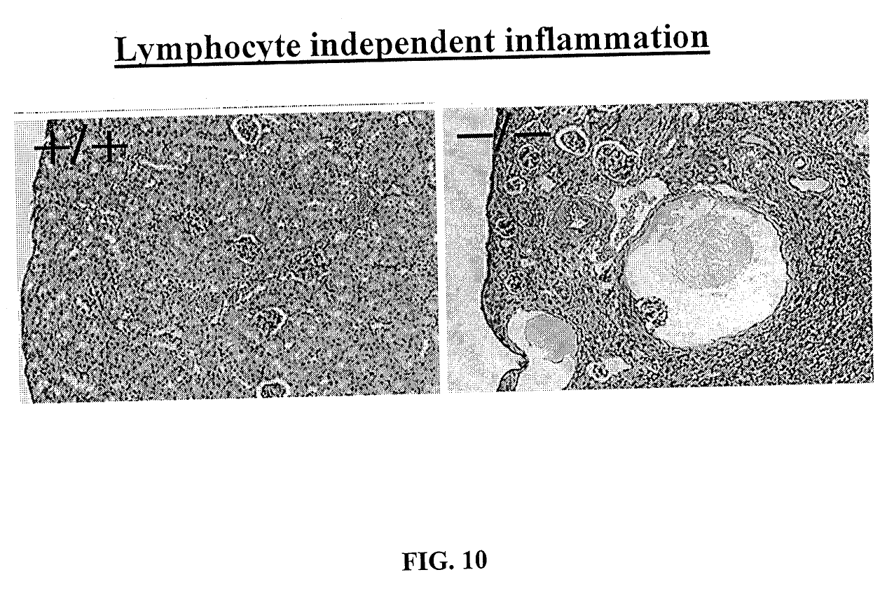

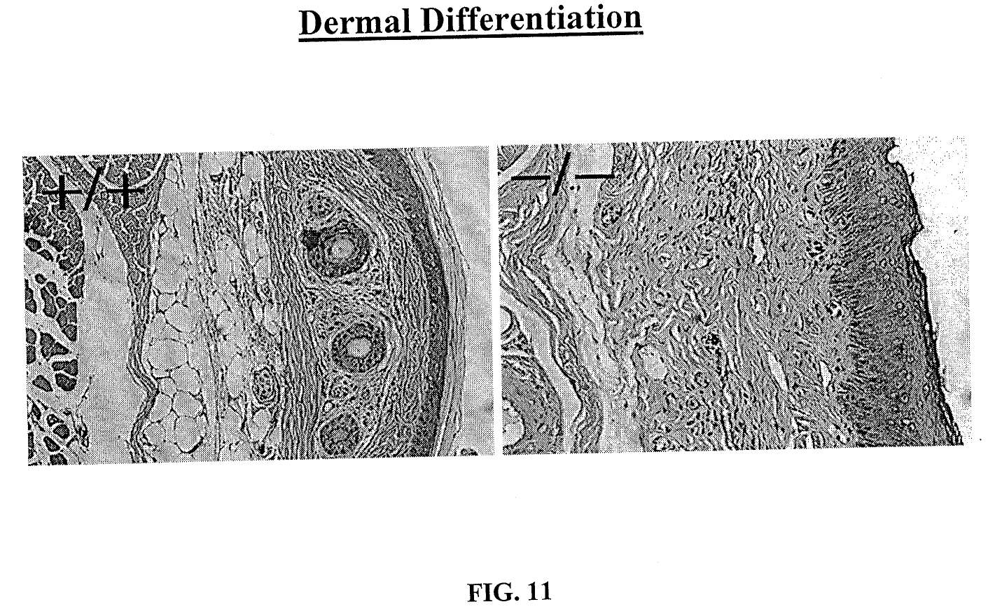

A20^{±} mice appeared normal without evidence of pathology. A20^{-/-} mice were born from interbred A20^{±} mice in Mendelian ratios, demonstrating that A20 is not required for embryonic survival. A20^{-/-} pups were runted as early as one week of age and began to die shortly thereafter (FIG. 3). Gross and histological examination of three to six week old A20^{-/-} mice revealed severe inflammation and tissue damage in multiple organs, including livers (FIG. 4, FIG. 5), kidneys (FIG. 6, FIG. 7), intestines (FIG. 8), joints and bone marrow (FIG. 9). Flow cytometric analysis of A20^{-/-} spleens and livers revealed increased numbers of activated lymphocytes (CD3⁺ CD44⁺), granulocytes (CD3⁻ Gr-1⁺ Mac-1⁺) and macrophages (CD3⁻ Mac-1⁺) (www.sciencemag.org/). Double mutant A20^{-/-} recombinase activating gene-1 deficient (RG-1^{-/-}) mice developed granulocytic infiltration, cachexia and premature death at a similar frequency and severity to A20^{-/-} RAG-1^{±} littermates (FIG. 7, FIG. 10), indicating that lymphocytes are not required for the inflammation seen in A20^{-/-} mice. Finally, skin sections revealed thickened epidermal and dermal layers without inflammation (FIG. 11). Thus, A20 is essential for preventing spontaneous innate immune cell mediated inflammation and tissue destruction, as well as regulating skin differentiation.

### Example 3

### Sensitivity of A20^{-/-} Thymocytes and MEFs to TNF Mediated PCD

The role of A20 in regulating inflammation was further evaluated by examining the sensitivity of A20^{-/-} mice to LPS. All A20^{-/-} mice died within two hours of injection of 5 mg/kg LPS, while A20^{+/+} and A20^{±} mice given 5, 12, or 25 mg/kg LPS survived without significant morbidity (Table 4). This hypersensitivity to LPS was correlated with increased numbers of A20^{-/-} splenocytes expressing TNF after LPS stimulation. In addition, A20^{-/-} mice were highly susceptible to low doses of TNF, as all A20^{-/-} mice died within two hours of injection of 0.1 mg/kg TNF, while A20^{+/+} and A20^{±} mice given 0.1, 0.2, or 0.4 mg/kg TNF survived (Table 4).

TABLE 4
A20^{-/-} mice succumb to sub-lethal doses of LPS and TNF*.

| | LPS mg/kg | | | TNF mg/kg | | |
|---|---|---|---|---|---|---|
| | 5 | 12.5 | 25 | 0.1 | 0.2 | 0.4 |
| +/+, +/− | 4/4 | 4/4 | 4/4 | 5/5 | 2/2 | 2/2 |
| −/− | 0/4 | — | — | 0/4 | — | 1 |

*17-20 day old A20^{+/+} and A20^{-/-} littermates were given the indicated doses of LPS or TNF. Numbers of mice surviving at two hours are indicated over the numbers of mice injected. Normal mice were observed for six hours further to rule out delayed effects of LPS or TNF.

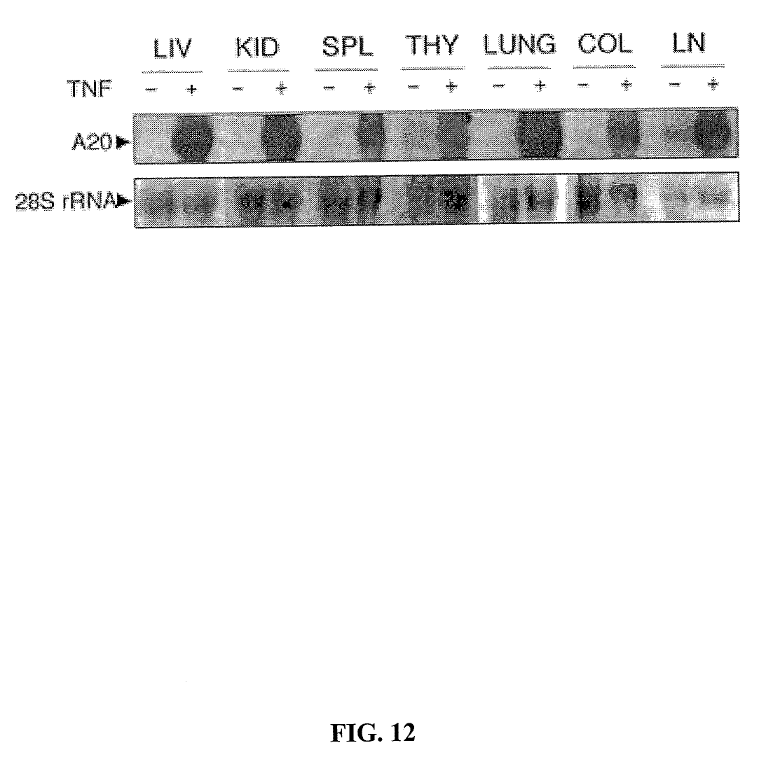

Consistent with the marked susceptibility of A20^{-/-} mice to TNF, A20 mRNA expression was dramatically increased by TNF in all tissues examined from normal mice (FIG. 12). Thus, A20 may protect mice from inflammatory mediators by regulating TNF responses in multiple cell types.

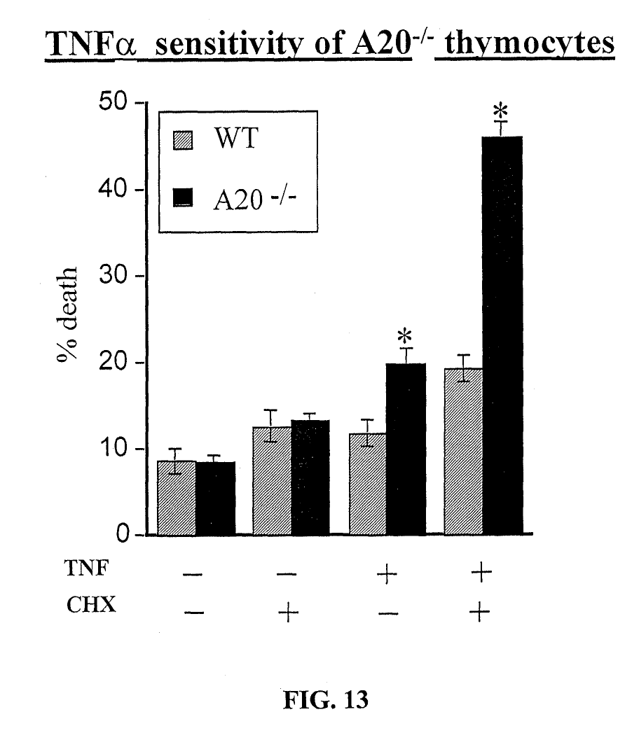

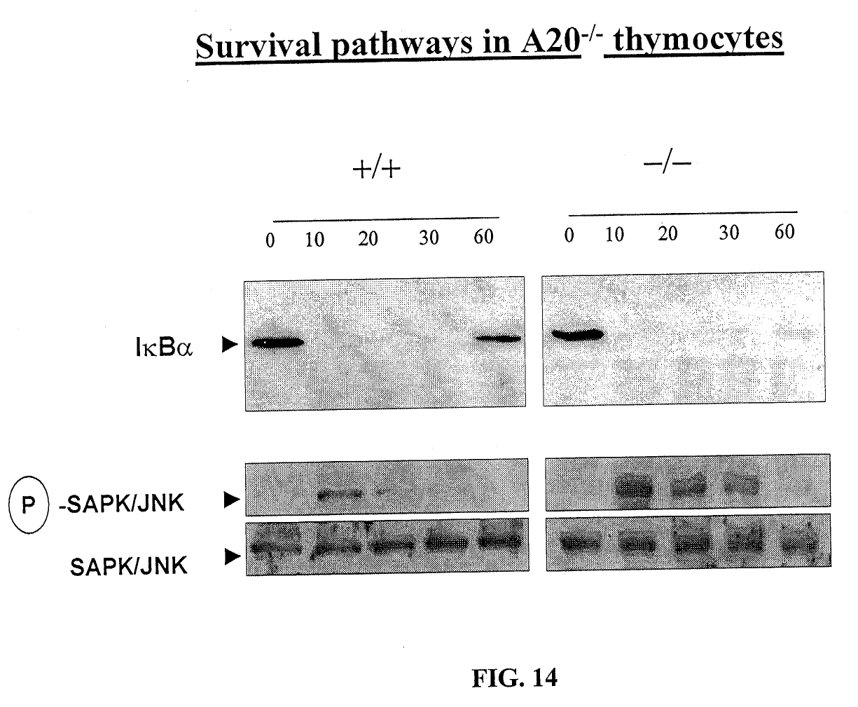

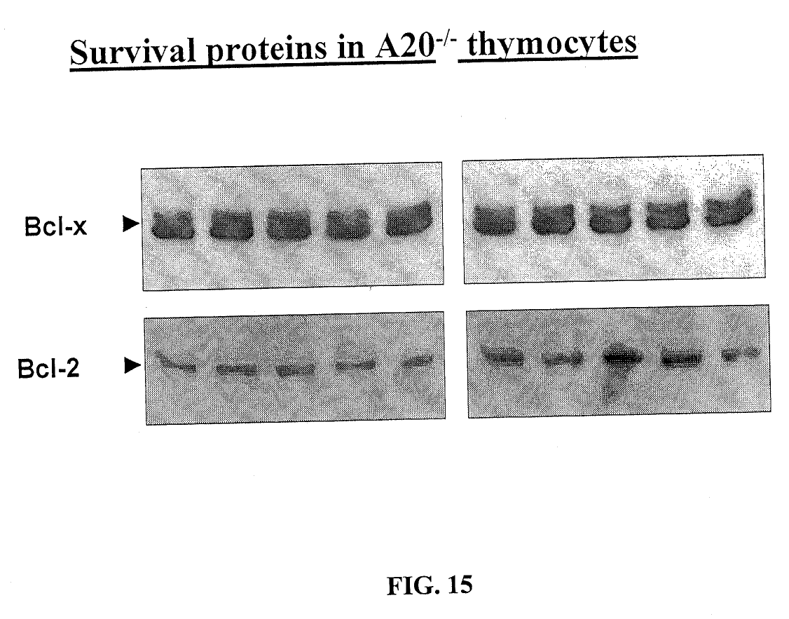

The hypersensitivity of A20^{-/-} mice to TNF may be due in part to A20's capacity to regulate PCD (Opipari et al., Tewari et al., 1995). Thymocytes constitutively express both TNF (Giroir et al., 1992) and A20 mRNA (Tewari et al., 1995). While corticosteroids, γ-irradiation, and Fas receptor ligation killed comparable numbers of A20^{±} and A20^{-/-} thymocytes, A20^{-/-} thymocytes were more sensitive to TNF, both in the presence and absence of cycloheximide (FIG. 13). TNF mediated PCD was blocked by the caspase inhibitor ZVAD-fmk, confirming that caspase dependent pathways kill these cells. Levels of the survival proteins Bcl-2 and Bcl-x were comparable in A20^{-/-} and A20^{+/+} thymocytes (FIG. 14). Both stress activated protein kinase (SAPK) (or c-Jun N-terminal kinase, JNK) phosphorylation and inhibitor of κB alpha (IκBα) degradation were seen in TNF treated A20^{-/-} thymocytes (FIG. 15), suggesting that the synthesis of survival proteins by SAPK/JNK and NF-κB dependent pathways was intact (Beg, 1996; Wang et al., 1996; van Antwerp et al., 1996; Liu et al., 1996). Thus, A20 protects thymocytes from TNF mediated PCD independently of protein synthesis or other known thymocyte survival factors.

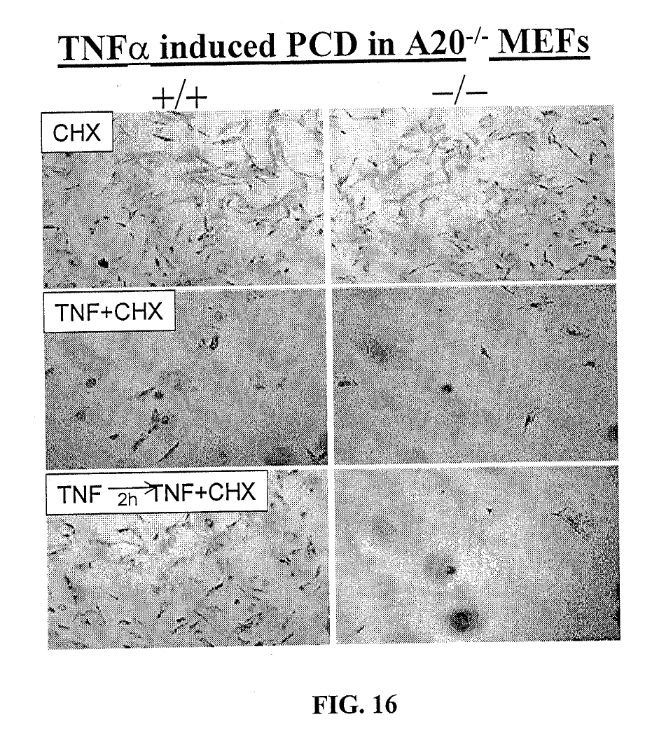

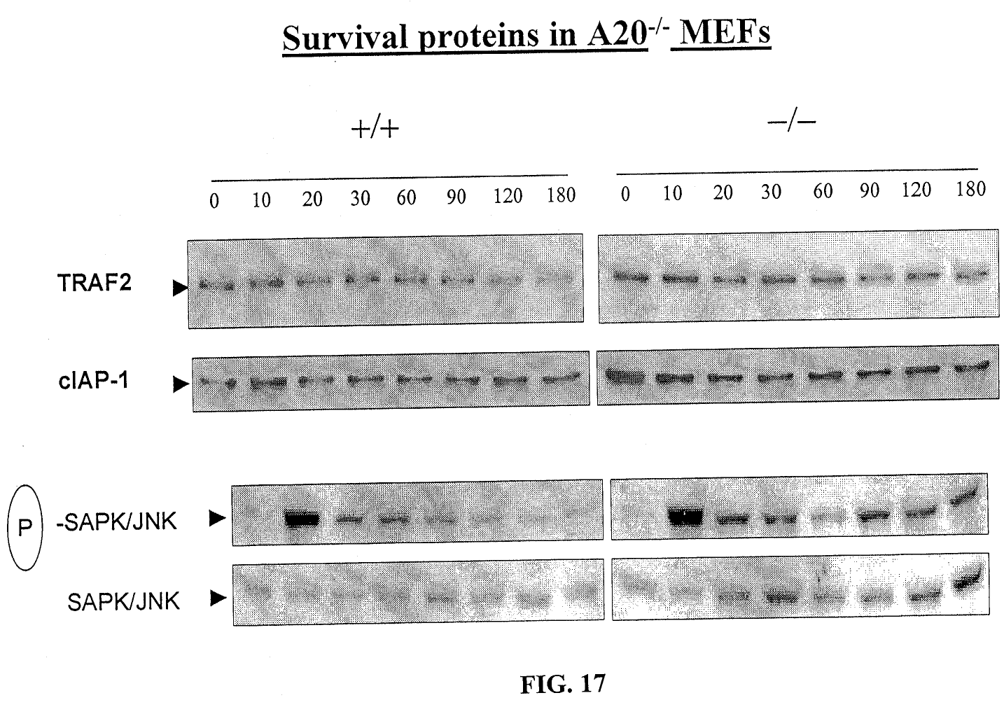

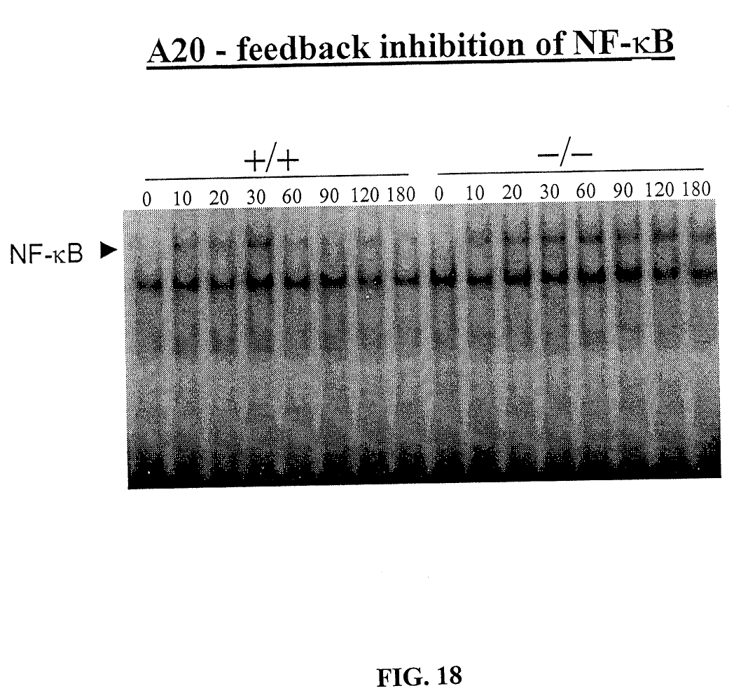

A20's ability to regulate TNF responses was further examined in embryonic fibroblasts (MEFs), which express negligible A20 mRNA at rest and dramatically increase levels of A20 mRNA expression after TNF treatment. While pretreatment of normal cells with TNF leads to the synthesis of survival proteins which protect these cells from subsequent TNF plus cycloheximide (Wong and Goeddel, 1994), A20^{-/-} MEFs universally died despite TNF pretreatment (FIG. 16). Activation of both SAPK/JNK and NF-κB pathways and similar levels of the survival proteins cellular inhibitor of apoptosis-1 (c-IAP1) and TRAF2 were seen in TNF treated A20^{+/+} and A20^{-/-} MEFs (FIG. 17 and FIG. 18). Thus, TNF mediated synthesis of presumably all NF-κB and SAPK/JNK dependent survival proteins (Wang et al., 1998) except A20 was insufficient to protect A20^{-/-} MEFs from TNF plus cycloheximide mediated PCD.

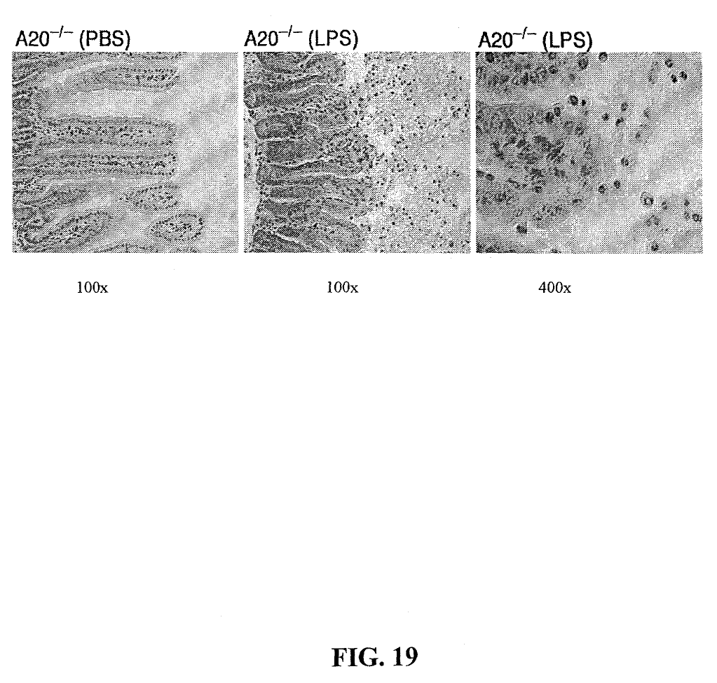

Histological analyses of intestines from these mice revealed profound inflammation, necrosis, hemorrhage, and epithelial cell sloughing in A20^{-/-} mice (FIG. 19). These dramatic results demonstrate that A20 restrains immune responses mediated by TNF, and prevents tissue damage normally induced by such stimuli. Moreover, the profound damage seen in A20^{-/-} intestines is qualitatively distinct and consistently more severe histologically than what was observed in other A20^{-/-} tissues, or in unperturbed A20^{-/-} mice. Hence, A20 appears to play a particularly critical role in regulating TNF responses in the intestine.

### Example 4

### Prolonged NF-κB Responses to TNF in A20^{-/-} MEFs

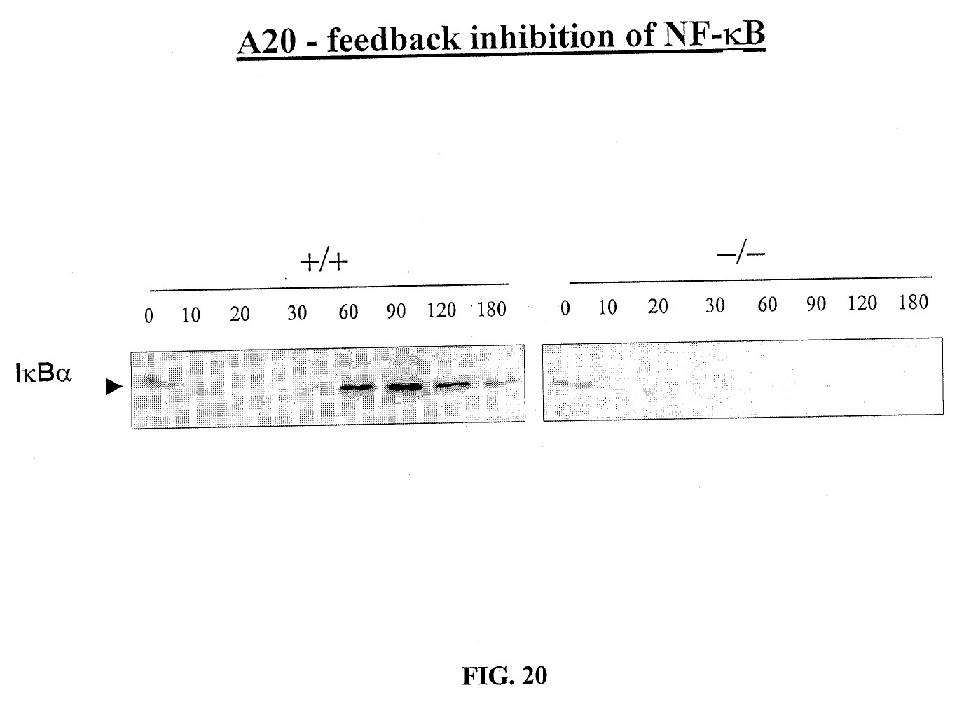

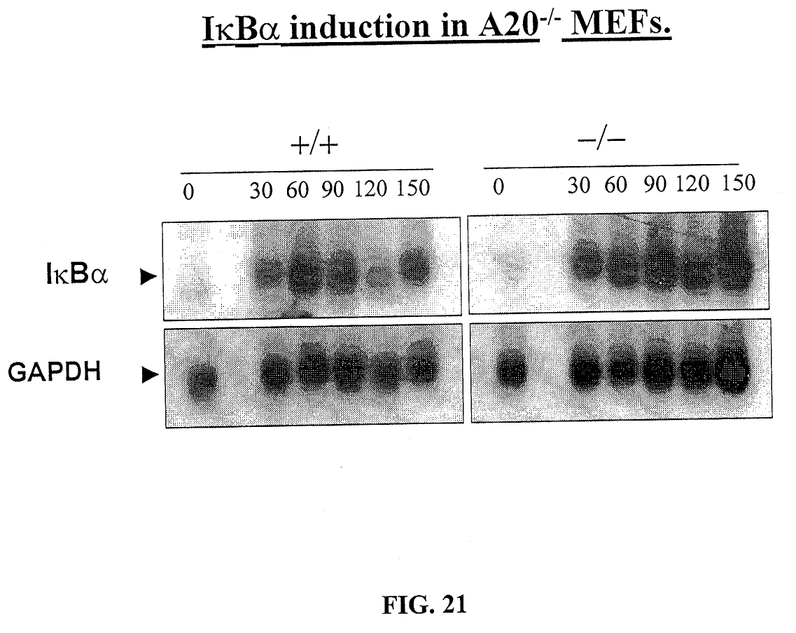

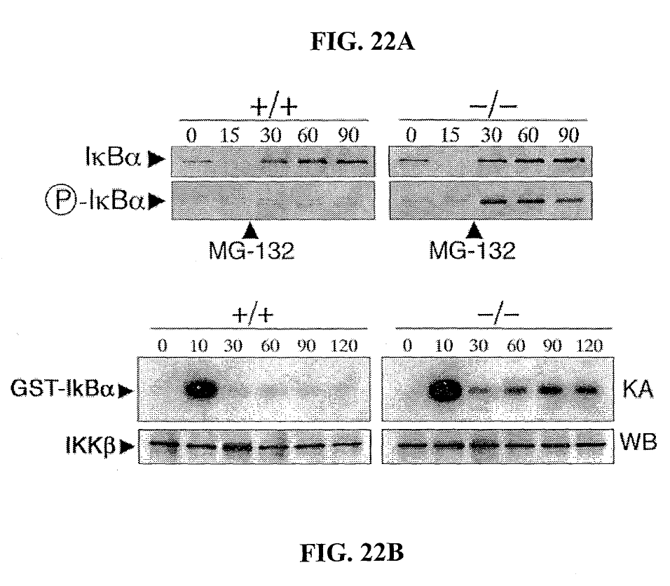

A20 inhibits NF-κB activation (Cooper t al., 1996), and dysregulated NF-κB activity leads to inflammation and premature death in IκBα^{-/-} mice (Beg et al., 1995). Moreover, the perturbed skin differentiation seen in A20^{-/-} mice resembles the skin of IκBα^{-/-} mice (Beg et al., 1996). Thus, the pathogenesis of A20^{-/-} mice may be due in part to dysregulated NF-κB activity. Repeated TNF treatment of normal MEFs caused IκBα degradation and NF-κB binding to DNA, followed by down-regulation of NF-κB binding and re-accumulation of IκBα protein by 60 min (FIG. 18 and FIG. 20). In contrast, NF-κB binding to DNA persisted and IκBα protein was not detected in A20^{-/-} MEFs from 60-180 min of TNF treatment (FIG. 18 and FIG. 20). IκBα mRNA levels, transcriptionally enhanced by NF-κB (Sun et al., 1993), increased in response to TNF in both A20^{+/+} and A20^{-/-} MEFs, indicating that the failure of A20^{-/-} MEFs to re-accumulate IκBα protein was not due to a failure to express IκBα mRNA (FIG. 21). Addition of the proteasome inhibitor MG-132 to MEFs 15 min after TNF treatment caused A20^{-/-} MEFs to regain normal levels of IκBα protein (FIG. 22A top panels), suggesting that the lack of IκBα protein re-accumulation in TNF treated A20^{-/-} MEFs was due to rapid degradation of newly synthesized IκBα protein, rather than the failure of these cells to translate IκBα mRNA. IκBα protein which re-accumulated in MG-132 treated A20^{-/-} but not A20^{+/+} MEFs was phosphorylated (FIG. 22A bottom panels), suggesting that persistent IKK (a multimeric complex comprised of IKKα, IKKβ, and IKKγ) activity caused rapid phosphorylation of newly synthesized IκBα protein in TNF treated A20^{-/-} MEFs. Direct measurement of IKK activity in lysates from TNF treated MEFs confirmed this suggestion (FIG. 22B). Therefore, synthesis of IκBα mRNA and IκBα protein is insufficient to terminate NF-κB signals in the absence of A20.

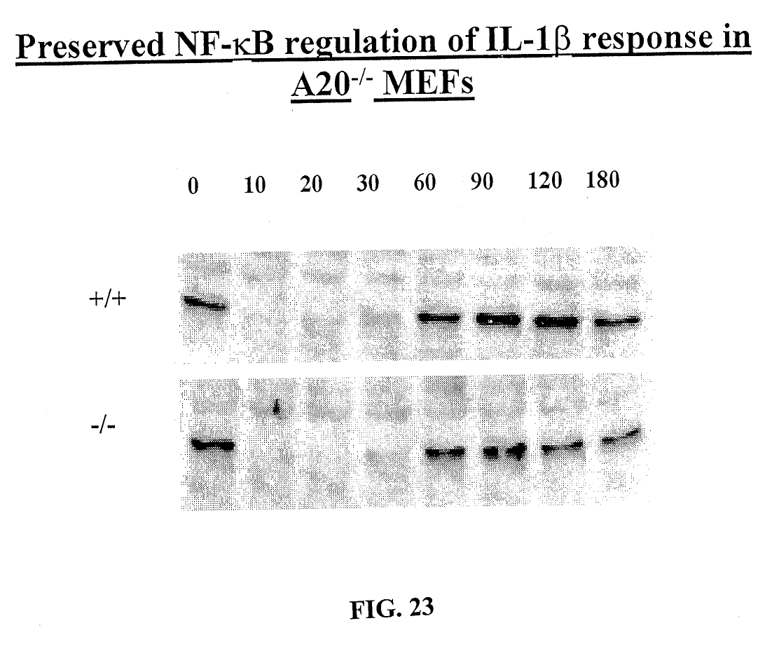

Finally, the role of A20 in regulating NF-κB responses to IL-1β was examined. NF-κB activity increased and decreased normally and IκBα protein re-accumulated normally in IL-1β treated A20^{-/-}MEFs (FIG. 23). Thus, while prior studies suggested that heterologous A20 can inhibit IL-1β induced NF-κB responses (Song et al., 1996; Jaattelaq et al., 1996), A20 is not essential for terminating these responses. Moreover, it is likely that A20 inhibits TNF activation of the NF-κB pathway upstream of IKKγ, since IKKγ is required for both IL-1β and TNF induced NF-κB activation (Rudolph et al., 2000).

### Example 5

### A20 Deficient Mice Develop Inflammation in the Absence of Lymphocytes

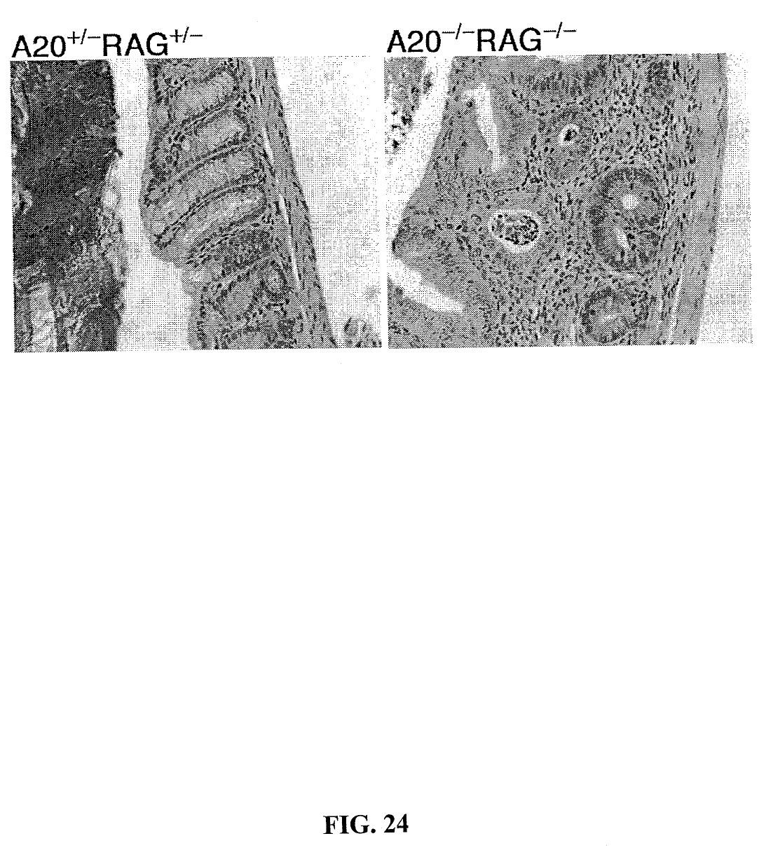

As A20 is constitutively expressed in lymphoid tissues of non-perturbed mice, it is possible that the profound inflammatory disease seen in A20^{-/-} mice may be due to dysregulated activity of lymphocytes or other immune cells. Indeed, most studies in experimental models of bowel inflammation to date have focused upon the role of T lymphocytes. To interrogate the role of A20^{-/-} lymphocytes in mediating inflammatory disease in these mice, A20^{-/-} mice were interbred with RAG-1^{-/-} mice which lack both T and B cells. Similar analyses of other colitis prone models have demonstrated a critical pathogenic role for T lymphocytes even when the targeted gene is expressed in non-lymphoid cells, such as IL-10^{-/-} mice (Davidson et al., 1996). By contrast, analyses of A20^{-/-} RAG-1^{-/-} double mutant mice along with A20^{-/-} RAG-1^{±} littermates reveals comparable morbidity and mortality, and colitis in both strains (FIG. 24).

Thus, despite the fact that A20 is selectively expressed in lymphoid tissues, bowel inflammation in A20^{-/-} mice can occur independently of lymphocytes. This finding distinguishes A20^{-/-} mice from prior models of immune mediated colitis (Kontoyiannis et al., 1999, Ma et al., 1995; Davidson et al., 1996). It also highlights the potential importance of A20 in regulating the activity of non-lymphoid immune cells (such as macrophages, dendritic cells, or granulocytes) and/or non-hematopoietic cells (such as intestinal epithelial cells).

### Example 6

### Adoptively Transferred A20^{-/-} Fetal Liver Stem Cells Cause Colitis in RAG-1^{-/-} Recipients

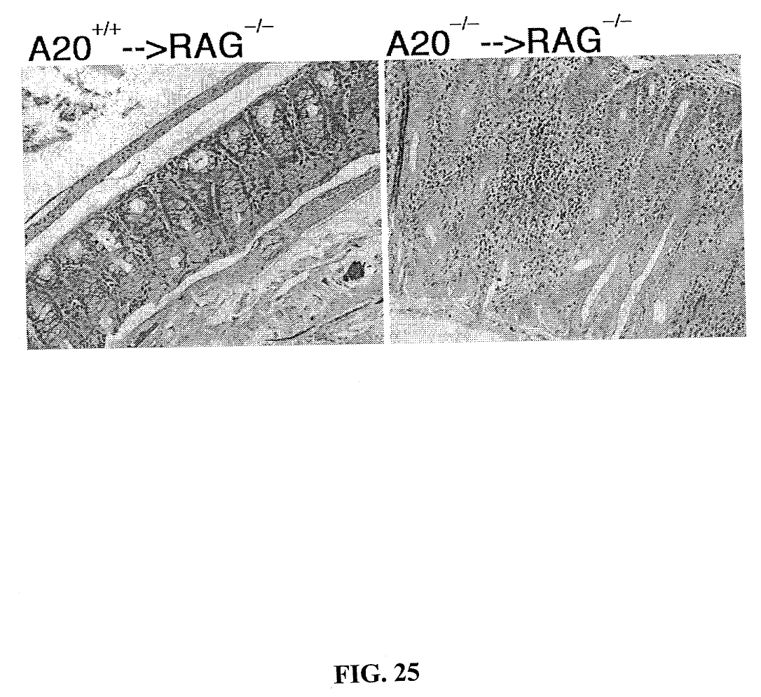

While the occurrence of inflammatory disease in A20^{-/-} RAG-1^{-/-} double mutant mice clearly demonstrates that lymphocytes are not required for the development of bowel inflammation, lymphocytes may nevertheless contribute to the perturbed immune homeostasis seen in A20^{-/-} mice. To interrogate the behavior of A20^{-/-} immune cells separately from diseased tissues comprised of non-hematopoietic A20^{-/-} cells, fetal liver cells containing hematopoietic cells were isolated from A20^{-/-} and A20^{-/-} E15.5 embryos and transferred into sublethally irradiated RAG-1^{-/-} mice. All lymphocytes from such reconstituted mice must be derived from the transferred fetal liver cells. The transfer of fetal liver cells guarantees that all transferred cells are naive when first transferred, and that allogeneic graft versus host immune responses can not occur, since adoptively transferred cells differentiate into mature lymphocytes entirely within lymphoid organs of the recipient RAG-1^{-/-} mice. Analyses of several of these chimeric mice reveals severe colitis in mice reconstituted with A20^{-/-} fetal liver stem cells, but not in those reconstituted with A20^{+/+} cells (FIG. 25). Thus, A20^{-/-} hematopoietic cells can cause colitis.

### Example 7

### A20 Protects Cells Against TNF Induced PCD

A20 protects some cell lines against TNF induced PCD in vitro (Tewari et al., 1995). Thus, it is possible that A20 may protect non-lymphoid cells against TNF secreted by inflammatory cells in vivo. Indeed, the widespread necrosis seen in tissues of A20^{-/-} mice could be partly due to the inability of those tissues to protect themselves from TNF. This possibility is also supported by the observation that A20 is dramatically induced by TNF in all non-lymphoid tissues tested (FIG. 12).

To interrogate the role of A20 in protecting cells against PCD, thymocytes were isolated from young, relatively healthy A20^{-/-} mice. Thymocytes constitutively express both TNF and A20 mRNA. A20^{-/-} thymocytes are comparably sensitive to normal thymocytes in response to glucocorticoids, Fas ligation, and γ-irradiation. However, A20^{-/-} thymocytes are more sensitive to TNF, with or without cycloheximide (Lee et al., 2000). Thus, A20 is essential for protecting thymocytes from TNF induced PCD, but not other forms of PCD induction.

As TNF induces A20 mRNA in non-lymphoid tissues, it was sought to investigate the role of A20 in protecting cells against PCD in non-lymphoid cells. For this purpose, murine embryonic fibroblasts (MEFs) were generated from E15.5 A20^{-/-} embryos. Like many other cells, MEFs are susceptible to TNF induced PCD in the presence of cycloheximide (CHX), or when NF-κB activation is blocked (Beg, 1996; Wang et al., 1996). Pretreatment of cells with TNF protects them from subsequent exposure to TNF plus cycloheximide because TNF pre-treatment is thought to lead to the synthesis of anti-apoptotic proteins via NF-κB dependent pathways. Accordingly, normal and A20^{-/-} MEFs were pretreated with TNF, and then treated them with TNF plus cycloheximide. While normal MEFs were protected by TNF pretreatment, all A20^{-/-} MEFs died (FIG. 16).

Thus, A20 is essential for protecting MEFs from TNF induced PCD despite the induction of other NF-κB dependent proteins, such as TNF receptor associated factor 1 (TRAF1), TRAF-2, and cellular inhibitor of apoptosis proteins (c-iaps) (Wang et al., 1998). Given the observation that A20 mRNA is dramatically induced by TNF in intestinal tissue, this study suggests that A20 may be induced to protect epithelial cells, endothelial cells, and/or other stromal cells in the intestine from TNF induced PCD. This protection may partly explain why intestines from A20^{-/-} mice display profound damage after in vivo exposure to TNF (FIG. 19).

### Example 8

### A20 Regulates TNF Induced NF-κB Responses

In addition to protecting cells against TNF induced PCD, A20 may inhibit TNF induced NF-κB activation (Song et al., 1996). NF-κB activates pro-inflammatory genes and may play multiple critical roles in different cell types which regulate intestinal homeostasis (Neish et al., 2000; Jobin and Sartor, 2000). Thus, the severe inflammation and tissue damage seen in A20^{-/-} mice may be partly due to unchecked NF-κB driven inflammatory responses to TNF. To investigate this possibility, the NF-κB responses were analyzed in two homogeneous cell populations: (i) thymocytes, which constitutively express A20 mRNA; and (ii) MEFs, which induce A20 mRNA after exposure to TNF. Similar findings were observed in both cell types, so MEFs were used for detailed kinetic studies to avoid any confounding issues of PCD in thymocytes (MEFs do not undergo PCD during the three hour time course of these studies). NF-κB activity was directly assessed by measuring nuclear protein binding activity to a conserved NF-κB DNA binding sequence in an electrophoretic mobility shift assay (EMSA). Such assays revealed induction of NF-κB DNA binding activity approximately 10 minutes after TNF treatment, followed by termination of this activity by 60 min (FIG. 14). The termination of NF-κB DNA binding activity in normal MEFs occurred despite repeated treatment with fresh TNF. By contrast, NF-κB DNA binding activity persisted in A20^{-/-} MEFs (FIG. 18). Thus, A20 appears to be essential for terminating TNF induced NF-κB activity.

While the mechanisms by which NF-κB DNA binding activity is normally terminated are incompletely understood, prior work has indicated that IκBα mRNA is transcriptionally activated by NF-κB (Sun et al., 1993). IκBα protein is then synthesized, binds to NF-κB, and inactivates NF-κB. The inventors therefore investigated the transcription and translation of IκBα. While IκBα mRNA is transcribed readily, IκBα protein does not reaccumulate in A20^{-/-} MEFs (FIG. 21 and FIG. 23).

This finding suggests that A20 may either regulate translation of IκBα mRNA, or prevent degradation of newly synthesized IκBα protein. A20 might perform the latter function by inhibiting the phosphorylation activity of the enzyme complex inhibitor of kinase kinase (IKK), or a more proximate step between TNFR and IKK. To distinguish between these potential functions for A20, TNF treated MEFs were treated with the proteosome inhibitor MG-132 fifteen minutes after TNF treatment. This dual treatment led to the reaccumulation of IκBα protein in A20^{-/-} MEFs, suggesting that IκBα protein is synthesized normally in the absence of A20, but is rapidly degraded by proteosome dependent pathways (FIG. 22A). As the rapid degradation of IκBα protein could be due to rapid phosphorylation of IκBα by IKK, IKK activity was interrogated in TNF treated MEFs by immunoprecipitating the IKK complex with an anti-IKKγ antibody and then measuring the capacity of this complex to phosphorylate a recombinant GST-IκBα (residue #1-54) substrate. This kinase assay demonstrates that IKK activity is indeed prolonged in A₂₀^{-/-} MEFs, compared to normal cells (FIG. 22B). Thus A20, itself induced by TNF, terminates TNF induced NF-κB signals by inhibiting IKK phosphorylation of IκBα. A20 is absolutely essential for this function, and is thus a critical regulator of inflammatory gene expression in vivo.

In view of the above, it was shown that A20 is an essential regulator of TNF induced NF-κB responses and cell survival in multiple cell types. The inventors have in fact discovered the first molecule that specifically down-regulates TNF induced NF-κB responses in vivo. The constant exposure of intestinal epithelium and immune cells to microbial organisms and the activation of innate immune cells by such agents likely leads to frequent, if not constitutive TNF expression. As TNF serum levels and baseline expression of TNF by myeloid cells appears normal in A20^{-/-} mice, the study suggests that the proper regulation of cellular responses to TNF is as important to mucosal immune homeostasis as regulation of TNF levels themselves. Moreover, NF-κB activity is critical to mucosal immune homeostasis (Jobin and Sartor, 2000; Sun et al., 1993), and elevated NF-κB dependent cytokine gene transcription is associated with human IBD as well (Neurath et al., 1998). Thus, the essential functions of A20 in terminating TNF induced NF-κB activation suggests that A20 is critical for regulating the expression of NF-κB dependent pro-inflammatory genes.

Having established the novel model of A20^{-/-} mice, it was made possible to determine A20 regulates the behavior of different cells in vivo, and examine how they each contribute to the regulation of mucosal immunity. The following Prophetic Examples detail studies that enable those of skill in the are to further understand the roles of A20 in regulating inflammation in the intestine. The creation of a novel strain of mice lacking the A20 gene, A20^{-/-} mice, provides compelling studies that A20 indeed serves these functions in vivo, and will allow those of skill to proceed in several ways. These studies will allow those of skill to confirm and extend the findings above in regard to knowledge as to the incidence and severity of bowel inflammation in A20^{-/-} mice and determine which cells and signals are dependent upon A20 for regulating intestinal inflammation.

### Example 9

### Evaluation of Spontaneous and TNF Induced Bowel Inflammation in A20^{-/-} Mice

A20 restricts cellular responses to TNF, and dysregulated TNF expression induces intestinal inflammation. The studies detailed above show that A20 is indispensable for regulating inflammation in vivo. Thus, it is possible to: a) determine whether intestines from unperturbed A20^{-/-} mice display histological, immunohistochemical and flow cytometric evidence of spontaneous inflammation; and b) determine whether intestines from A20^{-/-} mice display histological, immunohistochemical and flow cytometric evidence of inflammation after exposure to TNF.

### A. Determination as to Whether Intestines from Unperturbed A20^{-/-} Mice Display Histological, Immunohistochemical and Flow Cytometric Evidence of Spontaneous Inflammation

Dysregulated expression of TNF can induce intestinal inflammation, and A20 may restrict cellular responses to TNF. The studies further suggests that A20 is indispensable for regulating inflammation in vivo due to its ability to regulate TNF induced PCD and TNF induced NF-κB activity. Thus, to examine the potential role of A20 in regulating intestinal inflammation, A20^{-/-} mice were generated. The histological analyses reveal inflammatory lesions within both large and small intestines of multiple mice, so one will first systematically characterize the incidence and onset of inflammation in these mice. One will examine the correlation of intestinal inflammation with age of mice and with the presence of inflammatory indicators in other non-lymphoid (e.g., liver, kidney) and lymphoid tissues (e.g., spleen, peripheral lymph nodes) to determine if intestinal inflammation precedes or follows inflammation in other tissues. These studies will form the bases upon which subsequent cellular and genetic experimentation can be planned. These studies are done using standard histopathological as well as flow cytometric analyses. Formalin fixed sections of intestines from A20^{-/-} and A20^{+/+} mice are stained with hematoxylin and eosin (H&E) and examined for the presence of inflammatory cells as well as epithelial disturbance. A histological scoring system, based on previously described protocols (Corazza et al., 1999), is utilized to grade the severity of bowel inflammation: (a) leukocytic infiltration of the colon (0 to 3); (b) mucin depletion (0 to 2); (c) crypt abscesses (0 to 2); (d) epithelial erosion (0 to 2); (e) hyperemia (0 to 2); and (i) mucosal thickness (1 to 3). Summation of these scores will provide an index of the severity of disease along the length of the colon ranging from “no disease”=1 to “severe disease”=15. All specimens from a group will be collected, denoted with a code and examined by the same person on one day to prevent observer biases. This scoring system will provide a quantitative and objective means of comparing disease in different mice. To define the nature of infiltrating inflammatory cells, immunohistochemical studies will be conducted upon frozen sections of A20^{-/-} and A20^{+/+} intestines, using antibodies against CD3 (specific for T cells) and B220 (B cells). These immunohistochemical studies will be conducted primarily to confirm the qualitative involvement of immune cell subsets, rather than to quantitate subsets or activation status.

To quantitate the intestinal inflammatory infiltrates from A20^{-/-} mice, lamina propria monocytes will be extracted and assessed by flow cytometric analyses of surface markers including subset and activation antigens. Standard lymphoid and myeloid markers including CD3, CD4, CD8, B220 (T cell subsets and B cell marker), CD69, CD44 (activation markers), Mac-1 and Gr-1 (myeloid markers) will be used for these studies. Both lamina propria lymphocyte extractions and flow cytometric analyses are routinely conducted in the laboratory. To examine the functional status of infiltrating immune cells in A20^{-/-} mice, one will combine surface staining of extracted lamina propria lymphocytes with intracytoplasmic staining for cytokine expression. One will analyze these cells for the expression of cytokines IL-2, IL-5, IL-10, IL-12, TNF, and IFN-γ. Cytoplasmic staining for cytokine expression is also a well established technique in the laboratory.

These analyses will be important for two broad reasons. First, expression of these cytokines, combined with surface stains for CD4 and CD8 antigens, serves as a reflection of the differentiation of T cells towards TH1 versus TH2 type pathways. Biased differentiation of CD4⁺ T cells towards the expression of TH1 type cytokines has been associated with the predisposition of T cells to mediate bowel inflammation (Blumberg et al., 1999; Fiocchi, 1999). Secondly, as A20 appears to inhibit NF-κB activity in response to TNF signals, the selective expression of NF-κB dependent cytokine genes may indicate whether A20^{-/-} T cells display exaggerated NF-κB activity. Importantly, many TH1 type cytokines (IL-2Rα, IL-12, TNF, IFN-γ), and fewer TH2 type cytokines (IL-4) are known to be NF-κB dependent.

Intestinal inflammation is associated with perturbations in epithelial cells, including aberrant proliferation and cell death. These responses may reflect proximate effects of immune cell derived cytokines binding to receptors upon epithelial cells, direct interactions of immune cells upon epithelial cells, or aberrant responses of epithelial cells to cytokines. To determine whether epithelial cells are affected by inflammation in intestines from A20^{-/-} mice, one will examine H&E stained intestinal sections for signs of crypt hyperplasia and branching. To directly examine the proliferation index of epithelial cells, one will perform immunohistochemical BrdU labeling studies. In these studies, the inventors inject 1.5 mg of BrdU via intraperitoneal injection into A20^{-/-} and A20^{+/+} mice one hour prior to euthanizing mice for tissue harvest. Intestinal tissues are then fixed and incubated with anti-BrdU antibodies, which are subsequently developed with alkaline phosphate or peroxidase based techniques. Measurement of the number of BrdU⁺ cells per crypt or villus in comparable intestinal tissue sections will provide relative proliferation indices in A20^{-/-} and A20^{+/+} mice. The inventors have utilized this technique extensively to study proliferation of lymphocytes by flow cytometry, and have recently established this technology for studying proliferation in tissues as well.

To determine whether A20 protects epithelial cells from PCD in vivo, one will examine H&E histological sections for the presence of apoptotic bodies. One will then perform TUNEL stains on frozen sections of A20^{-/-} and A20^{+/+} mouse intestines. TUNEL stains involve the labeling of free DNA ends with fluorescent or biotin labeled dUTP molecules by terminal deoxynucleotidyl transferase (TdT), and the inventors routinely perform these stains using standard protocols with commercially available kits (e.g., see FIG. 3). The inventors can confirm whether TUNEL⁺ cells are immune versus non-immune cells by examining counterstained serial sections, and by performing dual color immunohistochemistry with antibodies directed against lymphoid specific surface markers (e.g., TCR, B220). By combining these histological, immunohistochemical and flow cytometric approaches, these studies should provide a thorough assessment of the spontaneous bowel inflammation in A20^{-/-} mice, as well as suggesting which cell types are pathogenic.

### B. Determination of the Response of Intestines from A20^{-/-} Mice to TNF

While the spontaneous development of bowel inflammation in A20^{-/-} mice suggests that A20 may mediate tonic inhibition of mucosal inflammation, the studies have also suggested that A20 is critically important in regulating inflammatory responses to TNF. First, A20 mRNA expression rises dramatically in intestinal tissues from normal mice injected with TNF. Secondly, intestines from the few TNF injected A20^{-/-} mice examined thus far display profound intestinal inflammation and epithelial damage. Thus, TNF binding to TNFR induces A20 synthesis, which may provide critical feedback inhibition of TNFR signals. In this scenario, the importance of A20 function would be most apparent after stimuli causing TNF secretion. Accordingly, one will examine the intestines of normal and A20^{-/-} mice after TNF injection. This model provides an additional and unique model for interrogating the roles of A20 in regulating intestinal TNF responses.

To examine the responses of A20^{-/-} mice to TNF, one will inject A20^{-/-} and A20^{+/+} mice with 0.1 mg/kg of TNF and histologically assess intestinal damage and PCD, as well as the expression of NF-κB dependent cytokines. This dose has been previously determined to be lethal for A20^{-/-} mice, while causing minimal morbidity in A20^{+/+} mice (Table 4). Since TNF injection causes dramatic induction of A20 mRNA in intestinal tissue within 30 minutes, one will euthanatize mice 30 min and one hour after injection to determine the consequences of A20's absence at time points when A20 is present at high levels. The importance of A20 expression at these time points is highlighted by the severe morbidity and mortality of A20^{-/-} mice soon after (two hours post TNF injection). Moreover, as A20^{-/-} mice survive through 90 minutes, these time points will allow harvesting of both control and A20^{-/-} animals at each time point. Yet another advantage of these studies will be that the acuity of these responses will greatly facilitate the ability to interpret the consequences of TNF exposure, i.e., significant cellular proliferation or differentiation should not occur in this time interval. Thus, the analyses the inventors perform are more likely to reflect direct actions of TNF upon cells the inventors are analyzing.

As the prior histological analyses of TNF injected mice reveal dramatic epithelial damage and inflammation in A20^{-/-} but not A20^{+/+} mice, one will examine these tissues using the same histological, immunohistochemical and flow cytometric assays described for studies of spontaneously diseased A20^{-/-} mice above. These studies will again address: the character and severity of the inflammatory immune cell infiltrate, as determined by flow cytometric analysis of extracted monocytes; the functional status of infiltrating immune cells as determined by cytoplasmic staining for cytokine expression; and the proliferative and survival responses of epithelial and other non-immune stromal cells in the intestinal milieu. Since non-immune stromal cells and epithelial cells appear to be selectively compromised in the first TNF injected A20^{-/-} mice the inventors have evaluated, one will pay particular attention to the integrity of these cells, and will evaluate PCD by TUNEL staining systematically in both small and large intestinal sections. Evidence of widespread stromal and epithelial cell PCD would suggest that these cells require A20 to protect them from TNF induced PCD. This would be a remarkable finding since virtually all cells are resistant to TNF induced PCD unless NF-κB pathways or protein synthesis are blocked (Beg, 1996; Wang et al., 1996).

The analysis of intestines from TNF injected A20^{-/-} mice directly interrogates the roles of A20 in regulating responses to TNF, and is an important complementary approach to the study of spontaneous inflammation in these mice. The one will use the results of baseline studies of spontaneous bowel inflammation described above to plan the age of mice used for these studies. The one will likely use 2-4 wk old mice. A potential confounding factor may be the spontaneous bowel inflammation which occurs in A20^{-/-} mice. This inflammation appears to be variable in severity, and appears to be histologically distinct, i.e., involve greater inflammatory infiltrate and less epithelial damage than intestinal damage seen in TNF injected mice. Thus, one will usually include PBS injected A20^{-/-} mice as controls in these studies. Finally, the technical aspects of evaluating epithelial damage in the intestine are well established in the laboratory, as described above.

### Example 10

### Interrogation of the Role of Lymphocytes in Bowel Inflammation in A20^{-/-} Mice

A20 is constitutively expressed in lymphoid tissues. Thus, A20^{-/-} lymphocytes may behave aberrantly, express excessive amounts of NF-□B dependent genes, and mediate inflammation in A20^{-/-} mice. To interrogate the role of lymphocytes in causing bowel inflammation in these mice, one may: 1) determine the incidence and severity of bowel inflammation in A20^{-/-} mice interbred with RAG-1 deficient (RAG-1^{-/-}) mice and 2) Determine the capacity of A20^{-/-} lymphocytes to mediate bowel inflammation after adoptive transfer into RAG-1^{-/-} mice.

A20 mRNA is constitutively expressed in lymphoid tissues, including intestinal lymphocytes in mesenteric lymph nodes. Hence, the absence of A20 may cause A20^{-/-} lymphocytes to respond aberrantly to TNF signals in vivo and contribute to autoimmune bowel inflammation. One will interrogate the potential roles of A20^{-/-} lymphocytes in mediating bowel inflammation in A20^{-/-} mice using two complementary approaches. First, one will analyze intestines from A20^{-/-} RAG-1^{-/-} double mutant mice; and secondly, one will adoptively transfer A20^{-/-} fetal liver hematopoietic cells into RAG-1^{-/-} mice.

### A. Determination of the Incidence and Severity of Disease in A20^{-/-} Mice Interbred with RAG-1 Deficient (RAG1^{-/-}) Mice

Elimination of adaptive lymphocytes (T and B lymphocytes) by interbreeding A20^{-/-} mice with RAG-1^{-/-} or RAG-2^{-/-} mice prevents the development of bowel inflammation in IL-2^{-/-}, IL-10^{-/-} and TNF^{ΔARE} mice (Kontoyiannis et al., 1999; Ma et al., 1995; Davidson et al., 1996). These findings are most likely due to aberrant activity of T cells in these models. As some consequences of dysregulated TNF expression are dependent upon adaptive lymphocytes (i.e., bowel inflammation) while others are not (i.e., arthritis) (Kontoyiannis et al., 1999), it will be important to directly examine the intestines of A20^{-/-} RAG-1^{-/-} double mutant mice to determine whether lymphocytes are required for the spontaneous colitis that occurs in A20^{-/-} mice. Accordingly, the inventors are interbreeding A20^{±} RAG-1^{±} with A20^{±} RAG-1^{-/-} mice, and one will analyze intestines from A20^{-/-} RAG-1^{-/-} double mutant offspring alongside A20^{-/-} RAG-1^{±} A20^{±} RAG-1^{-/-}, and A20^{±} RAG-1^{±} littermates. These analyses will involve similar histological, immunohistochemical and flow cytometric analyses as described above, except that one will focus on signs of non-lymphoid (i.e., myeloid) inflammatory cells and stromal or epithelial cell damage. Provocatively, the studies suggest that A20^{-/-} RAG-1^{-/-} double mutant mice display runting and die prematurely.

One will perform lamina propria monocyte extractions to determine the quantity and activation state of myeloid cells infiltrating the intestine, as the presence of these cells should not be affected by RAG-1 deficiency. One will examine the NK1.1 (natural killer), Mac-1⁺ (macrophage or dendritic cell), Gr-1⁺ (granulocyte) and Mac-1⁺ Gr-1⁺ (activated granulocyte) populations of cells in intestinal tissues from A20^{-/-} RAG-1^{-/-} double mutant and control mice and analyze these cells using cytoplasmic stains for pro-inflammatory cytokines such as TNF and IFN-γ. As discussed previously, TNF may be instrumental to the pathogenesis of bowel inflammation, and all these cell types are capable of producing and responding to TNF. If the inventors do not observe signs of intestinal inflammation in A20^{-/-} RAG-1^{-/-} double mutant mice, then adaptive lymphocytes—most likely T cells—are probably required for at least one step in the intestinal inflammatory cascade in A20^{-/-} mice.

T cells can mediate inflammation in several ways. First, they may induce B lymphocytes to secrete autoantibodies that cause inflammatory damage. Accordingly, one will interbreed A20^{-/-} mice with JH deficient (B cell deficient) mice to formally interrogate this possibility if A20^{-/-} RAG-1^{-/-} double mutant mice are disease free. Secondly, T cells may directly mediate tissue damage by elaborating cytotoxic molecules such as granzyme B and perforin. Thirdly, they may recruit innate immune cells by elaborating cytokines such as IL-12, IFN-γ and TNF. These stereotypic TH1 type cytokines appear to be pathogenic in several models of experimental colitis (Blumberg et al., 1999; Fiocchi, 1999). Finally, they may further contribute to dendritic cell activation via direct cell-cell interactions via molecules such as CD40. For example, when CD40 receptor on the surface of dendritic cells binds to CD40 ligand on the surface of activated T cells, both cells receive activation signals. All four of these processes are NF-κB dependent, since MHC class II molecules (which present antigenic peptides to B cells during B cell activation), perforin, IL-12, IFN-γ, TNF and CD40 are all NF-κB dependent genes. As the studies suggest that A20 terminates NF-κB responses, A20^{-/-} T cells may contribute to bowel inflammation by expressing supranormal levels of these genes. Indeed, the constitutive expression of A20 mRNA in lymphocytes suggests that A20 may serve to tonically inhibit lymphocyte expression of NF-κB dependent genes. Accordingly, one will directly interrogate the expression of these gene products on (and within) A20^{-/-} T cells, as well as A20^{-/-} myeloid cells. By combining histologic and flow cytometric studies of A20^{-/-} RAG-1^{-/-} double mutant mice with cytometric studies of A20^{-/-} T cells, the inventors should be able to elucidate potential pathogenic mechanisms by which A20^{-/-} T cells contribute to bowel inflammation. These studies will be complemented with further functional studies of A20^{-/-} T cells described below.

If the inventors observe signs of myeloid cell infiltration and activation in A20^{-/-} RAG-1^{-/-} double mutant mice that are comparable to those observed in A20^{-/-} RAG-1^{±} mice, then A20^{-/-} myeloid cells may become activated by innate immune stimuli and infiltrate tissues without assistance from T lymphocytes. These myeloid cells could behave aberrantly due to their inability to properly regulate responses to TNF. They may thus secrete supranormal amounts of IL-12, IFN-γ, and TNF, which would in turn recruit other inflammatory cells and cause tissue damage. Further studies to evaluate the pathogenic roles of A20^{-/-} myeloid cells are described.

A second important aspect of this analysis will be to examine the intestinal epithelial and stromal cell compartments of A20^{-/-} RAG-1^{-/-} double mutant mice for signs of hyperplasia or damage. During inflammation, epithelial cells may proliferate, become susceptible to programmed cell death stimuli, and/or undergo programmed cell death in response to stimuli such as TNF. Accordingly, as described, one will evaluate the integrity, proliferative response, and cell death of epithelial cells by examining H&E sections, and by performing immunohistochemical BrdU and TUNEL analyses. In all these studies, one will include A20^{-/-} RAG-1^{±} and A20^{±} RAG-1^{-/-} control littermates, which should be available from the same breedings which generate the A20^{-/-} RAG-1^{-/-} double mutant mice.

Importantly, the presence of comparable inflammatory infiltrates and tissue damage in intestines from A20^{-/-} RAG-1^{-/-} and A20^{-/-} RAG-1^{±} mice would provide evidence for a novel model of lymphocyte independent spontaneous colitis. Such inflammation might be mediated by myeloid cells such as macrophages, dendritic cells and/or granulocytes. This colitis would thus be distinct from the lymphocyte dependent colitis seen in IL-2^{-/-}, IL-10^{-/-}, and TNF^{ΔARE} mice (Kontoyiannis et al., 1999; Ma et al., 1995; Davidson et al., 1996). Indeed, the distinction from TNF^{ΔARE} mice may suggest that A20 regulates signals in addition to TNFR. Further dissection of the contribution of A20^{-/-} myeloid cells and A20^{-/-} non-immune stromal and epithelial cells to the pathogenesis of bowel inflammation in these mice will also be addressed by adoptive transfer studies.

The evaluation of spontaneous bowel inflammation in A20^{-/-} RAG-1^{-/-} double mutant mice will be conducted similarly to as described above. The histochemical and flow cytometric analyses of these mice should also be technically straightforward. The biological importance of these studies is quite significant, since positive evidence for lymphocyte independent bowel inflammation in these mice would make them quite distinct from existing immune models. As discussed below, further investigation of the roles of A20 in regulating T lymphocytes, myeloid cells, and intestinal epithelial stromal cells will be investigated in complementary investigations of purified lymphocytes and myeloid cells, and in adoptive transfer studies.

### B. Determine the Capacity of A20^{-/-} Hematopoietic Stem Cells to Mediate Bowel Inflammation after Adoptive Transfer into RAG-1^{-/-} Mice

Interbreeding A20^{-/-} and RAG-1^{-/-} mice will directly interrogate whether adaptive lymphocytes (T and B cells) are required for the development of bowel inflammation in A20^{-/-} mice. By contrast, the adoptive transfer of A20^{-/-} hematopoietic stem cells into A20 competent mice asks the question whether A20^{-/-} immune cells can contribute to this process. If A20^{-/-} RAG-1^{-/-} mice do not develop bowel inflammation spontaneously or in response to TNF, then lymphocytes may be critical for the pathogenesis of bowel inflammation in A20^{-/-} mice. In this case, further investigation of the pathogenic functions of lymphocytes will be performed to determine why they cause bowel inflammation (see below). If A20^{-/-} RAG-1^{-/-} mice do develop bowel inflammation, then lymphocytes are not required for the development of bowel inflammation. However, they may nevertheless contribute to this process. Moreover, this approach will help distinguish whether A20^{-/-} lymphocytes versus A20^{-/-} epithelial cells contribute more to bowel inflammation. Thus, regardless of the results of the analyses of A20^{-/-} RAG-1^{-/-} mice, one will adoptively transfer A20^{-/-} hematopoietic cells into A20 competent RAG-1^{-/-} mice to interrogate the capacity of A20^{-/-} lymphocytes to cause bowel inflammation. The recipient RAG-1^{-/-} mice used for these studies will bear the Ly5.2 congenic marker to allow distinction between donor (Ly5.1) and recipient (Ly5.2) cells. Since RAG-1^{-/-} mice are essentially normal mice with the sole exception that they lack B and T lymphocytes, reconstitution of these mice with A20^{-/-} hematopoietic cells will result in chimeric mice whose lymphocytes are entirely A20 deficient. Thus, these studies will provide a complementary approach towards understanding the potential role of lymphoid cells in contributing to bowel inflammation in A20^{-/-} mice.

To determine whether A20^{-/-} immune cells can mediate intestinal inflammation in an otherwise A20 competent intestine, one will perform two kinds of transfers of A20^{-/-} immune cells into RAG-1^{-/-} mice. The first approach is based on the observation that transfer of mature TH1 biased CD4⁺ T cells (e.g., CD45Rb^{Hi} CD4⁺ T cells) into syngeneic RAG-1^{-/-} or SCID mice causes colitis in recipient mice (Corazza et al., 1999). These studies will be conducted by purifying CD4⁺ T cells from A20^{-/-} and A20^{±} littermates (which the inventors routinely perform by negative selection using magnetic beads) and transferring them into RAG-1^{-/-} mice. Analysis of CD45Rb surface expression and cytoplasmic cytokine expression will be performed on cells prior to transfer. Immunohistochemical and flow cytometric analyses will be performed on intestinal tissues from recipient mice 6-12 weeks after transfer, as described. These studies require sufficiently inbred mice to avoid graft vs host reactions, which can also cause intestinal inflammation and thus confuse the interpretations of results from these studies. Thus, they will be performed in approximately one year after the A20^{-/-} mice have been backcrossed to C57B1/6 mice for a total of eight generations. (They are currently backcrossed two generations).

The second approach the inventors are taking is to transfer fetal liver cells−containing hematopoietic stem cells−from A20^{-/-} E16.5 embryos into RAG-1^{-/-} mice bearing the Ly5.2 congenic surface marker, and evaluate whether and to what degree intestinal inflammation occurs in recipient mice after a period of 6-12 wks. There are several important advantages for using fetal liver derived A20^{-/-} stem cells as donors for these studies. First, the possibility of graft versus host reactions are eliminated entirely, because no mature lymphocytes exist in E16.5 fetal livers (as opposed to bone marrows), and the development of A20^{-/-} stem cells from the C57B1/6×129 background within RAG-1^{-/-} mice on the C57B1/6 background causes all mature A20^{-/-} immune cells to recognize the recipient RAG-1^{-/-} cells as “self.” Meanwhile, the RAG-1^{-/-} recipient mice have no lymphocytes to mount an allogeneic host versus graft reaction. Secondly, this developmental tolerance and absence of graft versus host reactions allow performing these adoptive transfers with existing mice in the colony, rather than having to wait for syngeneic mice. Thirdly, the bone marrows of A20^{-/-} mice become infiltrated with inflammatory cells at a young age (as early as one week of age in some mice), so these bone marrows may not be a reliable source of stem cells as donors for such studies (Lodolce et al., 1998). Fourthly, the use of fetal liver stem cells—which contain no mature immune cells and are thus naive—avoids the possibility that donor cells are already activated prior to transfer.

To perform these studies, the inventors interbreed heterozygous male and female A20^{±} mice, check for vaginal plugs on the female mice on a daily basis, and thus obtain timed matings for dated embryos. Embryos are harvested on E16.5 (counting 0.5 day as the day of observation of the copulation plug), and individual fetal livers are dissected separately. While single cell suspensions of fetal liver are incubated at 37C, a fraction of each liver is lysed with proteinase K and subjected to PCR analysis using oligonucleotide primers the inventors have designed to allow rapid genotyping of A20^{-/-} mice. After genotyping each embryo, 2×10⁶ fetal liver cells from A20^{+/+} and A20^{-/-} embryos are injected into RAG-1^{-/-} mice. To facilitate engraftment of the donor fetal liver cells, all RAG-1^{-/-} mice receive 400 rads of gamma irradiation, a sublethal dose which the inventors have titrated for the RAG-1^{-/-} mice on a C57B1/6 background. Mice are then maintained in specific pathogen free microisolator cages and observed daily for the development of diarrhea or loose stools for six to twelve weeks. Any significantly wasted or moribund animals are sacrificed promptly. All other animals, including irradiated and non-reconstituted RAG-1^{-/-} control mice are sacrificed eight weeks after fetal liver transfer, and their intestines analyzed for the presence of immune cell infiltration and epithelial perturbation. As described above, histological and flow cytometric studies will be used for understanding the degree and type of inflammatory damage that occurs. An important feature of these analyses will be surface staining for the congenic Ly5.1/5.2 surface marker so that the inventors can distinguish Ly5.1 donor myeloid cells (which are A20^{-/-}) from Ly5.2 recipient myeloid cells (which are A20^{+/+}). This analysis will be combined with the cytoplasmic stains for IL-4, IL-10, IL-12, IFN-γ, and TNF cytokines.

Studies of this nature reveal that profound colitis occurs in RAG-1^{-/-} mice reconstituted with fetal liver cells from A20^{-/-} embryos (FIG. 11). The inventors thus anticipate that repeat studies will confirm this finding, and warrant further investigation of both phenotypic and functional studies of A20^{-/-} T and B cells. As described above, T cells mediate immune responses via multiple NF-κB dependent genes, including co-stimulatory membrane receptors, cytotoxic molecules, MHC proteins, cytokines and cytokine receptors. In addition, B cells activate NF-κB in response to signals from CD40, another TNF receptor family member. Since A20 interacts physically with TRAF2, which mediates signals from other TNF receptor family members, it is possible that A20 may regulate NF-κB responses to these other TNF receptor family members in addition to TNFR1 and TNFR2.

As the constitutive expression of A20 mRNA in normal lymphocytes may serve to restrict expression of NF-κB driven genes, one will directly examine the expression of these NF-κB dependent proteins on T and B cells freshly purified from A20^{-/-} and A20^{±} mice. Moreover, to further dissect the roles of A20 in regulating lymphocyte function, one will perform a series of in vitro activation studies with these cells. Specifically, one will stimulate T cells from lymph nodes from young (1-2 week old) A20^{-/-} and A20^{±} mice with anti-CD3 (as T cell receptor ligation may activate NF-κB) alone, or in combination with anti-CD28 (a co-stimulatory molecule required for CD30 expression on T cells), and/or anti-CD30 (a TNF receptor family member which can provide co-stimulatory signals to T cells via NF-κB activation) (Heath et al., 1999), or 4-IBB (another TNF receptor family member which can co-stimulate T cells via NF-□B activation) (Sica and Chen, 2000). In these studies, one will also evaluate the number and activation state of T cells by performing quantitative flow cytometric analyses of the NF-κB dependent proteins described above after 24, 48, and 72 hrs of culture. These studies will be coupled with BrdU proliferation studies (using in vitro BrdU labeling) and propidium iodide survival studies to determine the proliferative and survival of stimulated A20^{-/-} and A₂₀^{±} T cells. Similarly, one will stimulate purified splenic B cells from young A20^{-/-} and A20^{±} mice with IL-4 and anti-CD40 (a TNFR receptor family member which activates NF-κB and supports B cell proliferation and survival) (van Kooten and Bancherean, 2000) to interrogate the potential role of A20 in restricting B cell proliferation (as determined by in vitro BrdU proliferation studies) and immunoglobulin production (as determined by ELISA). In these ways, one will determine whether A20 normally restricts NF-κB responses to lymphoid specific signals from TNF receptor family members, and whether the absence of A20 allows unchecked NF-κB activity to lead to exaggerated lymphocyte proliferation and/or function.

The studies proposed in this example are complementary approaches to understanding the biology of A20^{-/-} immune cells. As noted above, the direct transfer of mature T lymphocytes must await further breeding to allow transfers that are free of confounding graft versus host reactions. Fortunately, the inventors have already established the alternative approach of fetal liver hematopoietic transfers, and have already acquired studies demonstrating that the inventors can successfully perform the timed matings, rapid PCR genotyping, and adoptive transfers required for subsequent analyses of reconstituted RAG-1^{-/-} mice. Histological and flow cytometric analyses of intestines from these chimeric animals are technically routine, as noted above. In addition to demonstrating the technical feasibility of these studies, these studies strongly suggest that A20^{-/-} hematopoietic stem cells mediate colitis in an otherwise A20 competent intestine. Hence, although this study could have provided only negative results, the studies provide a compelling argument that further in vitro studies with A20^{-/-} immune cells are warranted. These in vitro lymphocyte activation and analyses (e.g., quantitative FACS analyses of activation, proliferation and PCD) are routinely employed in the laboratory (Liu et al., 2000). As the development of colitis in these mice does not segregate the roles of A20^{-/-} lymphoid from myeloid cells, further alternative approaches towards understanding the biology of A20^{-/-} myeloid cells are discussed below.

### Example 11

### Examination of the Role of Myeloid Cells in Bowel Inflammation in A20^{-/-} Mice

Macrophages and other innate immune cells respond to TNF by elaborating additional pro-inflammatory cytokines. The studies suggest that myeloid cells from A20^{-/-} mice may elaborate supranormal levels of cytokines such as IL-12 and TNF when activated. To further evaluate the role of these cells in causing bowel inflammation in A20^{-/-} mice, one may: a) determine cytokine expression of both resting and stimulated A20^{-/-} macrophages, b) determine the response of A20^{-/-} RAG-1^{-/-} double mutant mice to TNF, c) determine the capacity of A20^{-/-} RAG-1^{-/-} hematopoietic stem cells (from fetal liver) to cause bowel inflammation after adoptive transfer into irradiated RAG-1^{-/-} mice.

Macrophages and other innate immune cells respond to LPS and TNF by elaborating additional pro-inflammatory cytokines, including TNF itself. Hence, macrophages may provide their own autocrine pro-inflammatory feedback loop in the absence of A20. As the study suggests that (i) A20^{-/-} RAG-1^{-/-} double mutant mice may develop runting and premature lethality (Lodolce t al., 1998), and (ii) myeloid cells accumulate in A20^{-/-} spleens (40), it is possible that myeloid cells from intestines of A20^{-/-} RAG-1^{-/-} mice play a major pathogenic role in the inflammation seen in A20^{-/-} mice. Hence, to evaluate the role of these cells in causing bowel inflammation in A20^{-/-} mice, one may pursue three approaches.

### A. Determination of Cytokine Expression of Both Resting and Stimulated A20^{-/-} Macrophages

Macrophages and dendritic cells mediate immune responses via the elaboration of multiple cytokines which regulate the activation, proliferation and differentiation of other innate and adaptive immune cells. As NF-κB selectively drives the expression of the TH1 type cytokines IL-12, TNF and IFN□, and not the TH2 type cytokines IL-10 and TGF-γ, the inventors may predict that A20^{-/-} macrophages may preferentially express TH1 type cytokines. Such cytokines might preferentially induce T cells to differentiate into TH1 type cells, a bias which is associated with bowel inflammation. One will thus isolate intestinal lamina propria and splenic monocytes from A20^{-/-} and A20^{+/+} mice, and perform cytoplasmic stains for these cytokines before and after TNF stimulation. Cytoplasmic stains are combined with surface stains to definitively identify sub-populations of cells expressing various cytokines in these assays, techniques that are described above and are well established in the laboratory. These initial studies will be complemented by the following functional studies describe below.

### B. Study the Response of A20^{-/-} RAG1^{-/-} Double Mutant Mice to TNF

As described in the studies, the inventors have noted that A20^{-/-} RAG-1^{-/-} mice develop spontaneous runting and cachexia. If the findings confirm that these mice develop spontaneous bowel inflammation, using analyses described above, then it is likely that innate immune cells such as macrophages, dendritic cells, and perhaps granulocytes play a critical role in mediating bowel inflammation in A20^{-/-} mice. Since these cells both respond to and secrete TNF, one will further interrogate the role of these cells in the novel acute model of TNF mediated colitis. As described above, exposure of A20^{-/-} mice to TNF causes rapid and profound changes in intestinal tissues which are dramatically different from the spontaneous inflammation seen in these mice. This important finding provides a unique means of investigating the roles of A20 in regulating TNF induced bowel inflammation in a highly reproducible and temporally controlled fashion.

To examine the role of A20^{-/-} myeloid cells in mediating inflammatory responses, one will inject TNF into A20^{-/-} RAG-1^{-/-} mice. Because A20^{-/-} mice become moribund within two hours of injection with TNF, one will first examine whether A20^{-/-} RAG-1^{-/-} mice develop the same gross clinical symptoms of labored breathing, reduced mobility, and collapse. This will reveal whether lymphocytes are required for this acute clinical response to TNF. The prediction would be that myeloid cells are more important than lymphoid cells in this response since innate immune cells typically represent the first line of defense and are the first responding inflammatory cells. One will next examine intestines from TNF injected A20^{-/-} RAG-1^{-/-} mice at 30 min, one hour, and two hours after injection, (and longer time points if clinically feasible) to harvest intestinal tissues for the same histological and flow cytometric analyses described above. Briefly, one will histologically evaluate the intestines of TNF injected mice, and will perform lamina propria extraction and flow cytometric studies to quantitate the myeloid cells extracted from A20^{-/-} RAG-1^{-/-} intestines. One will quantitate macrophages, dendritic cells and granulocytes. One will determine whether they express elevated levels of pro-inflammatory cytokines such as IL-12, IFN-γand TNF. One will also evaluate the intestinal epithelial cell layer and stroma for evidence of the epithelial cell damage seen in TNF injected A20^{-/-} mice, using TUNEL staining. In these studies, one will be able to rigorously determine whether lymphocytes are required for the acute TNF response in A20^{-/-} intestines. If the inventors confirm that they are not, then the aberrant response of A20^{-/-} intestines to T may be due to aberrant responses of A20^{-/-} myeloid cells, A20^{-/-} stromal cells, or both. To further distinguish these possibilities, one will directly interrogate the pathogenic role of A20^{-/-} myeloid cells with the adoptive transfer of these cells described below.

### C. Determination of the Capacity of A20^{-/-} RAG-1^{-/-} Fetal Liver Cells to Cause Bowel Inflammation after Adoptive Transfer into Irradiated RAG1^{-/-} Mice

To distinguish the potential pathogenic roles of hematopoietic A20^{-/-} myeloid cells from non-hematopoietic intestinal stromal cells, the inventors have devised a study in which A20^{-/-} myeloid cells can be purely and genetically isolated. To do this, one will interbreed A20^{±} RAG-1^{-/-} and mice to obtain A20^{-/-} RAG-1^{-/-} double mutant embryos (along with A20^{-/-} RAG-1^{±} and A20^{±} RAG-1^{-/-} control embryos). E16.5 embryonic fetal livers will be harvested, genotyped by PCR, and injected into sublethally irradiated (400 rads) Ly5.2 RAG-1^{-/-} recipient mice, as described above. The transferred cells will not cause any graft versus host reactivity because of their maturation within the RAG-1^{-/-} recipient mouse. The hematopoietic stem cells from the A20^{-/-} RAG-1^{-/-} double mutant embryos will only be able to generate A20^{-/-} myeloid cells when they mature in the recipient animals (since they will be RAG-1^{-/-} and hence unable to generate lymphocytes). Meanwhile, all stromal cells within the intestine will be A20 competent. Finally, the congenic Ly5.2 marker will allow distinguishing of donor Ly5.1 myeloid cells from recipient Ly5.2 cells.

Histological and flow cytometric analyses (as described above) of recipient RAG-1^{-/-} mice after 6-12 weeks will reveal whether A20^{-/-} non-lymphoid (myeloid) immune cells can cause intestinal inflammation in the complete absence of lymphocytes and in the presence of a normal intestinal stroma. Cytometric determination of the cytokines elaborated in myeloid cells extracted from these intestines will confirm their predilection for expressing TH1 type cytokines. Combining these analyses with surface lineage markers (Mac1, Gr-1, TCR), and Ly5.1, Ly5.2 will distinguish donor myeloid cells from recipient myeloid cells. Comparison of these RAG-1^{-/-} mice (which have only A20^{-/-} myeloid cells) with RAG-1^{-/-} mice that received A20^{-/-} RAG-1^{±} cells (which have both A20^{-/-} myeloid as well as A20^{-/-} lymphoid cells) will allow the inventors to further evaluate the relative contribution of lymphocytes versus monocytes to this process. If A20^{-/-} RAG-1^{-/-} hematopoietic cells do cause bowel inflammation in recipient RAG-1^{-/-} mice, then one will obtain definitive evidence of the pathogenic role of A20^{-/-} myeloid cells. This finding will highlight the importance of innate immune cells in sustaining chronic bowel inflammation, and will also provide a novel approach towards understanding myeloid cell mediated colitis.

If the inventors observe epithelial damage in A20^{-/-} RAG-1^{-/-} reconstituted RAG-1^{-/-} mice, this result would also imply that the cytokines elaborated by A20^{-/-} myeloid cells and/or cytotoxic factors secreted by granulocytes are sufficient to damage stromal cells despite normal regulation of TNF responses by A20 competent stromal cells. To further delineate the pathophysiology of dysregulated myeloid cell function, one will evaluate the acute response of these novel reconstituted chimeric mice to injected TNF. This evaluation will involve the same histological and flow cytometric studies described above. In this temporally controlled fashion, one will be able to precisely dissect the aberrant in vivo response of A20^{-/-} myeloid cells in terms of the factors they secrete, as well as how these cells affect non-hematopoietic cells in the intestine.

As one of the mechanisms by which macrophages mediate inflammatory responses is via the secretion of cytokines which direct differentiation of T cells towards a TH1 type pathway, it is possible that A20^{-/-} RAG-1^{-/-} double mutant myeloid cells may cause colitis only when T cells are present. Thus, if A20^{-/-} RAG-1^{-/-} hematopoietic cells do not cause disease after transfer into RAG-1^{-/-} mice, or after transfer and TNF injection, one will perform an study in which normal T cells are reconstituted along with A20^{-/-} myeloid cells. To do this, one will co-inject A20^{-/-} RAG-1^{-/-} double mutant fetal liver cells and normal A20^{+/+} or A20^{±} fetal liver cells into irradiated RAG-1^{-/-} mice. Again, because both types of donor cells will be hematopoietic stem cells which mature within the host, no graft versus host (or graft versus graft) reactions should occur. This study will thus reveal whether these A20^{-/-} myeloid cells cause intestinal inflammation in the presence of a full complement of normal lymphoid cells and normal stromal intestinal cells.

These studies will directly interrogate the ability of A20^{-/-} myeloid cells (from A20^{-/-} RAG-1^{-/-} stem cells) to cause bowel inflammation in A20 competent recipients. Their ability to do so in the presence or absence of normal lymphocytes will be examined using mixed chimera reconstitution. One potential pitfall is the presence of normal myeloid cells in recipient RAG-1^{-/-} mice. Thus, one will conduct parallel studies using lethally irradiated C57B1/6J mice instead of sublethally irradiated RAG-1^{-/-} mice as recipients. Lethal irradiation should eliminate all host myeloid cells well before the time that recipient mice are analyzed. Moreover, one will have the benefit of cell surface Ly5.1/5.2 markers to document the relative contribution of host versus donor myeloid cells Finally, in the course of other studies, one will also be generating a tissue specific/conditional knock-out of the A20 gene. These mice will ultimately provide an independent mechanism to interrogate the roles of A20 in regulating distinct cell types, and thus shed further light on which cells are pathogenic in the inflammatory damage seen in A20^{-/-} mice.

### Example 12

### Examination of the Role of TNF Signals in Mediating Inflammation in A20^{-/-} Mice

A20 inhibits TNF induced activation of NF-κB and protects cells from TNF induced programmed cell death (PCD). To determine whether TNF signals via TNFR1 or TNFR2, or whether other TNFR family member receptors are essential for inciting spontaneous inflammation and tissue damage in A20^{-/-} mice, one may: a) determine the incidence and severity of spontaneous bowel inflammation in A20^{-/-} mice treated with a TNFR-immunoglobulin fusion protein and b) determine the severity of bowel inflammation in A20^{-/-} mice interbred with either TNF^{-/-}, TNFR1^{-/-}, or TNFR2^{-/-} mice.

A20 regulates TNF induced NF-κB responses, and protects cells against TNF induced PCD. The molecular mechanism by which A20 performs these functions are unclear, but may be related to A20's ability to interact with TRAF2, a TNFR signaling molecule. TRAF2 is also thought to mediate signals from other TNFR family receptors, including CD30, CD40 and others. Thus, while the inventors have a great deal of studies suggesting that A20 is essential for properly regulating TNF responses, A20 may also regulate signals from other pro-inflammatory receptors, such as the IL-1 receptor (Song et al., 1996). To determine whether A20 regulates other pro-inflammatory signals, one will directly block TNF signals in A20^{-/-} mice using both cellular and genetic approaches.

### A. Determining the Incidence and Severity of Spontaneous Bowel Inflammation in A20^{-/-} Mice Treated with TNFR-Ig

The studies suggests that A20^{-/-} mice are exquisitely sensitive to heterologous TNF. Thus, the spontaneous bowel inflammation seen in these mice may be due entirely to the inability of A20^{-/-} mice to properly terminate cellular responses to TNF secreted in response to endogenous bacterial antigens. To investigate potential roles for A20 in regulating TNF signaling in vivo, one will treat young (two week old) A20^{-/-} mice with a soluble TNFR-immunoglobulin (TNFR-Ig) fusion protein (Iizuka et al., 1999). This TNFR-Ig is a relatively stable molecule which acts by binding to soluble TNF and which blocks TNF signaling in vivo for periods of up to several weeks. Isotype matched control immunoglobulin will be injected into control animals. This will be done with several litters of mice, as the onset and incidence of bowel inflammation in unmanipulated A20^{-/-} mice is somewhat stochastic. Mice will thus be observed daily, and mice that are moribund will be sacrificed along with control littermates. All mice will be euthanatized no later than six to eight weeks of age, and analyzed histologically and flow cytometrically as described. Improvement of the severity of bowel inflammation in TNFR-Ig treated A20^{-/-} mice, as compared to untreated mice, will suggest that endogenous TNF is pathophysiologically important in these mice. Lack of improvement may suggest that stimuli other than TNF lead to unchecked NF-κB responses, but this result may be more difficult to interpret, as the inventors can not be sure that all endogenous TNF is neutralized by TNFR-Ig.

This study possesses the advantage that these reagents are immediately available. A significant amelioration of bowel inflammation in TNFR-Ig fusion protein (but not control Ig) injected mice will indicate that TNF signals are the predominant pathogenic signals regulated by A20. The major potential pitfall of these studies is not having an independent way of positively confirming blockade of endogenous TNF signals in the setting of no clinical amelioration of disease. Thus, a negative result from this study will not be conclusive, so one will also utilize the alternative approach of genetically blocking TNF signals as described below.

### B. Determining the Severity of Bowel Inflammation in A20^{-/-} Mice Interbred with TNF^{-/-}, TNFR1^{-/-}, or TNF2^{-/-} Mice

To evaluate the potential role of TNF signaling in the pathogenesis of bowel inflammation in A20^{-/-} mice, one will interbreed A20^{-/-} with TNF^{-/-} mice, and evaluate A20^{-/-} TNF^{-/-} double mutant mice alongside A20^{-/-} TNF^{±} mice as well as the other relevant control mice. The critical branch point is whether A20^{-/-} TNF^{-/-} double mutant mice are completely normal, or whether they develop at least some spontaneous inflammation. If A20^{-/-} TNF^{-/-} mice do not develop any bowel inflammation, then it is likely that the regulation of TNF signals by A20 is the exclusive molecular abnormality relevant to this disease process, and one will accordingly focus entirely on TNFR signals. TNF binds to cells via two receptors, TNFR1 and TNFR2. It is unclear which receptor (if either) would be preferentially regulated by A20. While most TNF signals in vivo have been thought to be mediated via TNFR1, both TNFR1 and TNFR2 appeared to be required to mediate bowel inflammation in TNF overexpressing TNF^{ΔARE} mice (Kontoyiannis et al., 1999; Kollias et al., 1999). Moreover, recent evidence indicates a more complex interplay between these receptors (Chan et al., 2000a; Chan et al., 2000b). TNFR2 signaling may modulate TNFR1 signals and shift the dominant signaling of TNFR1 engagement away from TRAF2 and NF-κB activation and towards FADD mediated PCD (Chan et al., 2000). A20's interactions with TRAF2 may differentially regulate these two pathways emanating from TNFR1 receptors. These and other evolving biochemical complexities arising from detailed studies of TNFRs (Chan et al., 2000) indicate that one will be obliged to systematically study A20^{-/-} TNFR2^{-/-} mice in parallel with A20^{-/-} TNFR1^{-/-} mice, rather than presume specific functions for each receptor.

If A20^{-/-} TNF^{-/-} mice are disease free, one will interbreed A20^{-/-} with TNFR1^{-/-} and TNFR2^{-/-} mice to genetically dissect the roles of TNFR1 and TNFR2 signals in mediating inflammation in A20^{-/-} mice. One will evaluate both A20^{-/-} TNFR1^{-/-} and A20^{-/-} TNFR2^{-/-} double mutant mice (along with the relevant A20^{-/-} and A20^{±} littermates which should be available from the breedings) for the spontaneous development of bowel inflammation, using the histological and flow cytometric assays described above. One will also interrogate the acute response of both A20^{-/-} TNFR1^{-/-} and A20^{-/-} TNFR2^{-/-} double mutant mice to TNF injection, as described.

The presence of normal intestinal mucosa in either A20^{-/-} TNFR1^{-/-} or A20^{-/-} TNFR2^{-/-} double mutant mice would implicate the corresponding TNFR as a required component for mediating bowel inflammation in A20^{-/-} mice. Coupled with disease free A20^{-/-} TNF^{-/-} mice, the presence of bowel inflammation in either A20^{-/-} TNFR1^{-/-} or A20^{-/-} TNFR2^{-/-} double mutant mice would implicate the remaining receptor (not eliminated by gene targeting) as the pathogenic signal in the absence of A20 regulation. As noted above, TNFR1 and TNFR2 contribute differently to the activation of NF-κB versus PCD responses to TNF. TNFR2 is thought to activate NF-κB activity via TRAF2 in the absence of TNFR1, and TNFR2 is not thought to recruit TRADD and FADD death signaling proteins independently of TNFR1. Thus, to further investigate how each receptor contributes to pathology in the absence of A20, one will both analyze the expression of NF-κB dependent proteins and test for PCD by TUNEL staining in intestines from either diseased A20^{-/-} TNFR1^{-/-} or A20^{-/-} TNFR2^{-/-} double mutant mice. Moreover, one will examine NF-κB and PCD responses in homogenous populations of cells (e.g., thymocytes and lymphocytes) extracted from these mice. Specifically, one will examine NF-κB activity and the expression of NF-κB dependent proteins in these cells at various timepoints after in vitro TNF stimulation (as described above). One will also interrogate the survival of A20^{-/-} TNFR1^{-/-} and A20^{-/-} TNFR₂^{-/-} cells in response to TNF and cycloheximide treatment. These biochemical studies will then be correlated with immunohistochemical assays of NF-κB dependent proteins and TUNEL stains of intestines from the same mice. In these ways, one will be able to correlate the biochemical roles of A20 with the physiological consequences in vivo.

If A20^{-/-} TNF^{-/-} mice do develop at least some bowel inflammation—even partially ameliorated inflammation—then signals other than TNF are likely to be regulated by A20 in vivo. This is an important distinction from antibody or fusion protein mediated interference with signaling molecules. A partial result is clearly interpretable in a genetic study such as this where no protein can be made. Among other pro-inflammatory signals which activate NF-κB in innate immune cells, IL-1 is frequently associated with TNF secretion. The studies suggest that IL-1 mediated activation of NF-κB is terminated normally in A20^{-/-} MEFs, so A20 may not be required for termination of IL-1 signals. This finding indicates that A20 does not regulate all NF-κB activity. The difference between A20 regulation of TNF signals versus IL-1 signals may be related to A20's ability to interact with TRAF2, a signaling molecule which associates with TNFR1 and TNFR2, but not the IL-1 receptor. Thus, one will study the response of cells from A20^{-/-} TNF^{-/-} mice to other TNFR receptor family members which may share TRAF2, such as CD30, CD40, and TRAIL. Indeed, some of this information will be available from studies of A20^{-/-} lymphocytes proposed above. Once A20^{-/-} TNF^{-/-} mice are available, cells from these mice will be optimal for interrogating these responses, since there will be no TNF secreted by these cells to induce aberrant responses secondarily. Agonist ligands are available for all these molecules and will be tested on A20^{-/-} TNF^{-/-} cells before interbreeding A20^{-/-} mice with mice lacking these genes.

These studies will directly interrogate the roles of A20 in regulating various signals known to activate NF-κB and PCD. Again, if A20^{-/-} TNF^{-/-} mice develop at least some bowel inflammation, significant effort will be shifted towards the interrogation of other pro-inflammatory signals—particularly mediated by other TNFR family members—which might be negatively regulated by A20. The one should be able to interrogate all of these signals using cellular (and in most cases, genetic) approaches.

### Example 13

### A20, TNF and Innate Immune Responses

### A. Evaluating the Roles of A20 in Regulating Innate Immune Responses

TNF stimulates innate immune cells during inflammatory responses and A20 restricts cellular responses to TNF in multiple cell types. Preliminary data suggests that A20 is important for regulating the activation and expansion of innate immune cells in vivo. Thus, in order to further elucidate this the inventors will: a.) determine whether A20^{-/-} RAG-1^{-/-} double mutant mice develop comparable inflammation in the absence of A20^{-/-} adaptive lymphocytes; b.) determine whether chimeric mice reconstituted with hematopoietic fetal liver stem cells from A20^{-/-} or A20^{-/-} RAG-1^{-/-} double mutant mice develop inflammation in the presence of normal stromal tissues; c.) examine the homeostasis of innate immune cells, including macrophages, granulocytes and dendritic cells in A20^{-/-} and A20^{-/-} RAG-1^{-/-} mice; and d.) study functional responses of purified A20^{-/-} innate immune cells to pro-inflammatory stimuli.

### B. Determining the Mechanism(s) by Which A20 Regulates TNF Induced NF-κB Signaling

TNF activates genes associated with cellular activation via the activation of NF-κB signaling pathways. Preliminary studies suggest that A20 may be essential for terminating TNF induced NF-κB activity. To determine the mechanisms by which A20 performs these functions, the inventors will: a.) determine which proximate TNFR signaling molecules associate with endogenous A20 protein; b.) determine whether proximate TNFR signaling molecules associate differently in A20^{+/+} and A20^{-/-} MEFs, and correlate these biochemical events with IKK kinase and NF-κB activity; and c.) determine whether A20 regulates NF-κB activation by stimuli other than TNFR1.

### C. Determining Whether and how A20 Regulates TNF Induced JNK Signaling

TNF stimulates cellular proliferation via the activation of JNK signaling pathways, which lead to activation of c-Jun. While NF-κB dependent pathways are thought to terminate JNK signaling, initial data surprisingly indicates that A20^{-/-} cells may display prolonged JNK signaling despite persistent NFκB signaling. Thus, A20 may independently regulate JNK signaling. To demonstrate that A20 is essential for regulating c-Jun activity in vivo, the inventors will: a.) determine whether c-Jun activity is prolonged in A20^{-/-} cells; b.) determine the mechanism by which A20 regulates JNK signaling; and c.) Determine whether excessive c-Jun activity leads to hyperproliferation of A20^{-/-} cells

### Example 14

### Evaluating the Roles of A20 in Regulating Innate Immune Cell Responses

### A. A20^{-/-} Mice Develop Severe Inflammation

A20^{-/-} mice spontaneously develop, inflammation, runting and cachexia, and many mice die prematurely. A20^{-/-} mice also spontaneously develop inflammation in multiple organs, characterized by mononuclear and granulocytic infiltrates in virtually all mice examined (FIG. 5, FIG. 7). These findings suggest that A20 is a critical negative regulator of inflammation in vivo.

### B. A20 is Expressed Constitutively in Lymphoid Tissues, and is Dramatically Induced in all Tissues

Widespread expression of TNFRs indicates multiple cell types can respond to TNF in vivo. The role of A20 in regulating TNF responses would be restricted to cells that express A20. Prior studies suggested that A20 was expressed selectively in lymphoid tissues (Tewari et al., 1995). As A20 mRNA is induced by TNF, the inventors assessed A20 mRNA expression in tissues from unperturbed as well as TNF injected mice. These studies confirmed the constitutive expression of A20 mRNA in thymi and lymph nodes, suggesting that T lymphocytes may constitutively express this gene (FIG. 12). Surprisingly, TNF also dramatically induced A20 mRNA within one hour in all tissues tested (FIG. 12). This finding suggests that A20 may regulate TNF signals in multiple cell types during inflammatory responses.

### C. Both Adaptive and Innate Immune Cells Spontaneously Infiltrate Tissues in A20^{-/-} Mice

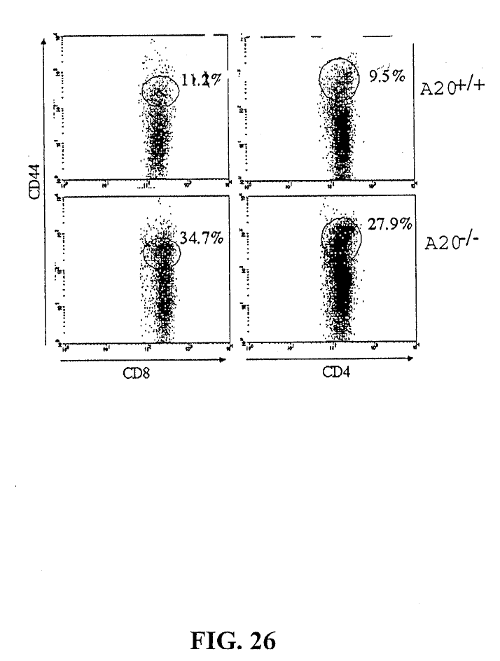

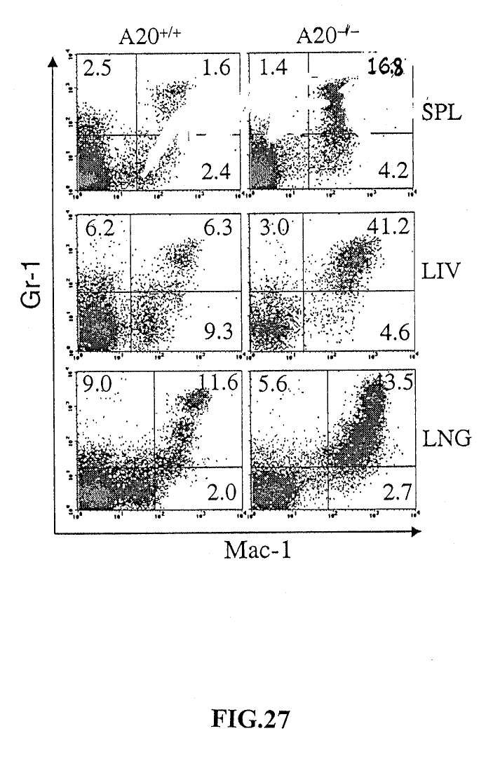

The pleiotropic expression of both A20 and TNF suggests that A20 may regulate both innate and adaptive immune cell responses. The inventors investigated the nature of inflammatory cells that accumulate in A20^{-/-} mice and found that these cells include increased numbers of activated lymphocytes (FIG. 26) and myeloid cells (FIG. 27).

Moreover, analyses of sera from A20^{-/-} mice revealed dramatically (two to ten fold) elevated levels of IgM, IgG1, IgG2a, IgG2b and IgA isotype immunoglobulins, indicating that spontaneous B cell activation occurs in these mice as well.

### D. A Novel Antiserum Recognizes A20 Protein in Several Immune Cell Lineages

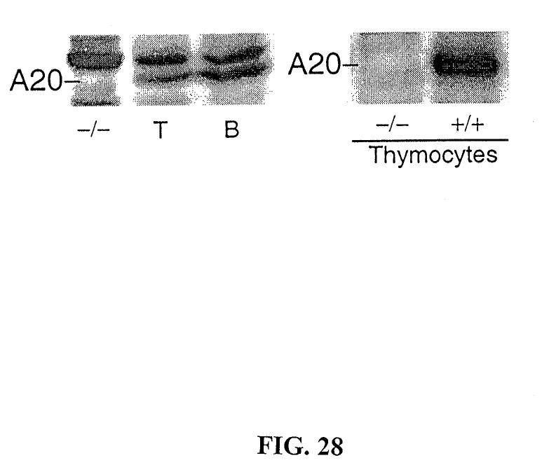

Mixed inflammatory infiltrates in tissues of A20^{-/-} mice suggest that A20 may regulate the homeostasis of several immune cell lineages. Because innate and adaptive immune cells participate in extensive intercellular communication, aberrant function of one cell lineage may affect the function of multiple other lineages. To better understand the role of A20 in regulating specific cell lineages, the inventors have developed a novel polyclonal antiserum to measure murine A20 protein levels. This antiserum recognizes an 82 kD band present in normal but not A20^{-/-} cells. This band is selectively competed by the cognate peptide to which the antiserum was raised, but not irrelevant peptides. Preliminary studies with this antiserum suggest that A20 protein is constitutively expressed in purified B and T lymphocytes as well as macrophages (FIG. 28).

### E. A20^{-/-} Mice Develop Inflammation in the Absence of Adaptive Lymphocytes

As A20 appears to be constitutively expressed in adaptive lymphocytes, and as spontaneous activation of T and B cells occurs in A20^{-/-} mice, the inventors examined the requirement for A20^{-/-} T and B lymphocytes in mediating inflammatory disease by interbreeding A20^{-/-} mice with RAG-1^{-/-} mice. Analyses of A20^{-/-} RAG-1^{-/-} double mutant mice revealed that significant morbidity and mortality develops spontaneously in these mice. Examination of these mice revealed that large numbers of myeloid cells accumulate in tissues from A20^{-/-} RAG-1^{-/-} (but not A20^{±} RAG-1^{-/-}) mice.

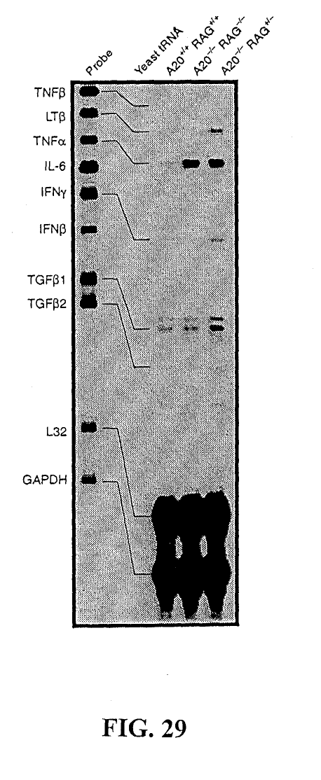

Interestingly, preliminary molecular analyses of diseased tissues from A20^{-/-} RAG-1^{-/-} and A20^{-/-} RAG-1^{+/+} littermates suggests that the inflammatory infiltrate is qualitatively distinct in these two strains of mice. In addition to the predictable absence of lymphocytes in A20^{-/-} RAG-1^{-/-} mice, RNAse protection analysis reveals dramatically elevated levels of TNF in both strains, while elevated levels of IFNγ, LTβ and TGFβ1 are seen in A20^{-/-} RAG-1^{+/+} but not A20^{-/-} RAG-1^{+/+} mice (FIG. 29).

Excessive production of TNF—an NF-κB dependent gene—is a common molecular event in these inflamed tissues, suggesting that the inability of A20^{-/-} cells to properly regulate TNF responses leads to further production of TNF. These findings also suggest that T and/or B lymphocytes either directly elaborate or induce synthesis of IFNγ, LTβ and TGFβ1 as part of a broad inflammatory response in A20^{-/-} mice, but that these factors are not required for myeloid inflammation and morbidity. Thus, despite the fact that A20 appears to be constitutively expressed in T and B cells, inflammation in A20^{-/-} mice can occur independently of these cells. Moreover, A20 is essential for regulating innate immune homeostasis. This finding also indicates that A20 plays critical roles in regulating the function of non-lymphoid cells, including myeloid immune cells (e.g., macrophages, dendritic cells, or granulocytes) and/or non-hematopoietic cells (e.g., endothelial, stromal, and epithelial cells).

### F. Adoptively Transferred A20^{-/-} Fetal Liver Stem Cells Cause Myeloid Inflammation in Normal Mice

Non-hematopoietic stromal cells influence the development and function of immune cells in multiple ways. For example, the development of T and B cells is aberrant in IκBα^{-/-} mice but normal in chimeric mice reconstituted with IκBα^{-/-} fetal liver stem cells (Chen et al., 2000), suggesting that extrinsic (non-hematopoietic) NF-κB signals regulate immune cell development. Moreover, NF-κB dependent genes in endothelial cells and stromal cells include adhesion molecules and chemokines that regulate activation and tissue infiltration of mature immune cells. As A20 is expressed in both hematopoietic and non-hematopoietic cells, the autoimmune disease seen in A20^{-/-} mice may be related to aberrant development or function of A20^{-/-} hematopoietic or A20^{-/-} stromal cells, or both. To examine the behavior of A20^{-/-} immune cells separately from non-hematopoietic A20^{-/-} cells, fetal liver hematopoietic cells were isolated from A20^{-/-} and A20^{+/+} E15.5 embryos (C57BL/6J-129 mixed Ly5.1⁺ background) and transferred into lethally irradiated C57BL/6J-SJL mice bearing the Ly5.2⁺ marker. Embryos were genotyped by PCR and fetal liver stem cells were transferred within six hours of tissue harvest. The transfer of fetal liver cells ensures that allogeneic graft versus host responses do not occur. Analyses of these chimeric mice eight to twelve weeks after transfer reveals that all mice reconstituted with A20^{-/-} fetal liver cells display inflammatory infiltrates in multiple organs, runting and cachexia, while chimeric mice reconstituted with A20^{+/+} cells remained essentially normal. Thus, A20^{-/-} stem cell derived hematopoietic cells appear to exhibit functional defects leading to autoimmunity—in the presence of normal stromal and endothelial cells.

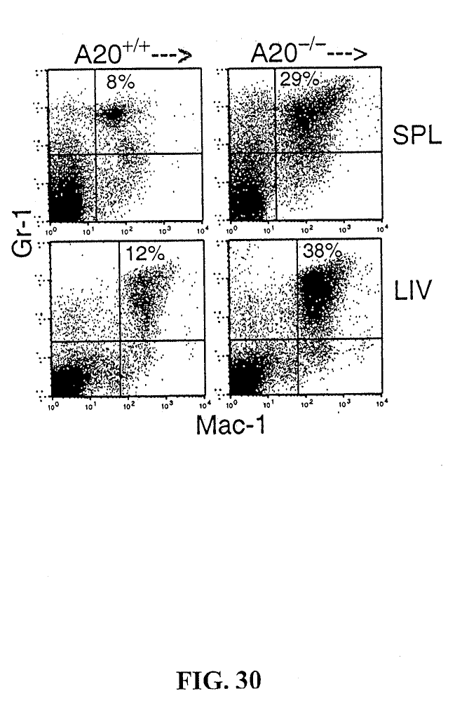

Preliminary analyses of peripheral blood from these chimeric mice reveal higher percentages of granulocytes from mice reconstituted with A20^{-/-} fetal liver stem cells. Consistent with these findings, dramatically expanded populations of Mac-1⁺ Gr-1^{Hi} (activated granulocytes) and Mac-1⁺ Gr-1^{Int} F480⁺ (macrophages) cells were noted in spleens, livers, and multiple other tissues (FIG. 30).

By contrast, the number of donor stem cell derived A20^{-/-} T and B cells was markedly reduced compared with donor A20^{+/+} T and B cells. These findings suggest that A20^{-/-} granulocytes and macrophages may expand aberrantly due to cell-autonomous defects in these innate immune cells.

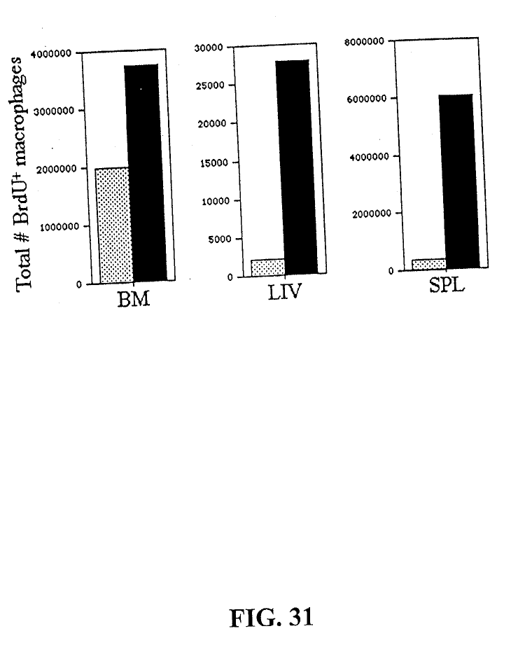

Accumulation of these cells may reflect increased proliferation and migration of these cells into tissues, or reduced death of these cells, or both. To better understand why these populations of cells expand in vivo, the inventors examined the cells proliferative index by injecting chimeric mice with BrdU three hours prior to sacrifice. This short term “pulse” labeling of mice determines the number of cells traversing S phase during a limited time. Cells cannot proliferate twice during such a short time frame, so no dilution or loss of BrdU from continuously dividing cells can occur. Analysis of BrdU incorporation into Mac-1⁺ F480⁺ cells (macrophages) revealed that dramatically elevated (two to ten fold) numbers of BrdU⁺ cells were found in spleens and livers of chimera reconstituted with A20^{-/-} stem cells compared with chimera reconstituted with A20^{+/+} stem cells (FIG. 31).

These data indicate that markedly increased numbers of macrophages are cycling in A20^{-/-} reconstituted chimera. Similar findings were obtained in intact A20^{-/-} mice. While increased cell survival may also contribute to the accumulation of these cells, the relatively low turnover of macrophages under normal conditions (i.e., relatively few macrophages proliferate or die within a three hour period in normal mice) argues that increased cycling plays a significant (and perhaps dominant) role. The increased number of BrdU⁺ cells in bone marrows suggests that increased production of these cells occurs in vivo, and increased numbers of BrdU⁺ cells in spleens and livers suggest that increased proliferation of mature macrophages may occur as well.

Granulocytes are short lived effector cells, whose numbers in tissues are largely regulated by signals that stimulate proliferation and demargination of bone marrow precursors. Such signals also induce migration of activated cells into tissues expressing appropriate adhesion and homing molecules. Preliminary studies indicate that increased numbers of BrdU⁺ Gr-1^{Hi} Mac-1⁺ cells (granulocytes) are present in bone marrows and spleens from chimera reconstituted with A20^{-/-} fetal livers than in control chimera. Thus, increased granulocyte precursor proliferation and activation may contribute to the marked expansion of these cells in A20^{-/-} fetal liver reconstituted chimera.

As stromal and endothelial cells in these chimeric mice are normal, granulocyte expansion and tissue infiltration are ultimately due to primary abnormalities of hematopoietic cell function. Thus, hematopoietic cell derived signals (e.g., macrophage derived TNF, or T cell derived GM-CSF) and/or increased sensitivity of A20^{-/-} myeloid cells to such ligands may cause aberrant granulopoiesis. To begin to evaluate the potential contribution of such factors to inflammation in these chimeric mice, RNAse protection analyses (RPA) of mRNA derived from tissues from these mice were performed. These studies indicate that TNF, LTβ, IFNγ and TGFβ1 mRNA expression levels are increased in tissues from chimeric mice reconstituted with A20^{-/-} but not A20^{+/+} stem cells. These NF-κB dependent genes largely overlap with those that are increased in inflamed tissues from intact A20^{-/-} mice (FIG. 29). Combined with the similar character of cellular infiltrates, this finding suggests that similar inflammatory mechanisms occur in both intact A20^{-/-} mice and chimeric mice reconstituted with A20^{-/-} fetal liver stem cells. Specifically, aberrant NF-κB activity in A20^{-/-} myeloid cells may drive autoimmune inflammation.

### G. Adoptively Transferred A20^{-/-} RAG1^{-/-} Fetal Liver Stem Cells cause Inflammation in Normal Mice

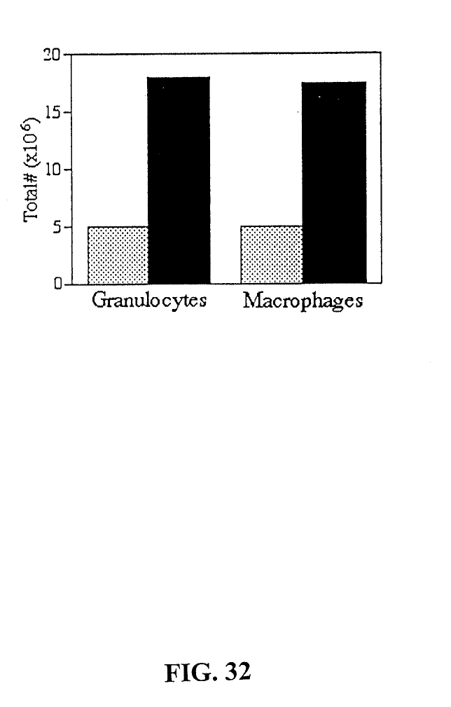

The selective expansion of myeloid cells in chimeric mice receiving A20^{-/-} fetal liver stem cells suggests that these cells possess aberrant cell-intrinsic responses to normal environmental signals. However, it remains possible that A20^{-/-} T cells may influence A20^{-/-} myeloid cells in these mice through intercellular communications such as CD40-CD40L, IL-17-IL-17R, or other interactions including soluble factors (e.g., GM-CSF). Indeed, these signals can be bi-directional in several situations. To further understand whether A20^{-/-} myeloid cells expand aberrantly in the absence of T cell derived signals, the inventors have interbred A20^{-/-} and RAG-1^{-/-} mice, and adoptively transferred fetal liver stem cells from A20^{-/-} RAG-1^{-/-} double mutant embryos into lethally irradiated Ly5.2⁺ mice. The resulting chimeric mice contain A20^{-/-} myeloid cells in an otherwise A20 competent environment. Preliminary analyses of these mice reveal approximately three fold increased numbers of granulocytes and macrophages in mice receiving A20^{-/-} RAG-1^{-/-} stem cells than in mice receiving A20^{+/+} RAG-1^{-/-} stem cells (FIG. 32). Thus, A20^{-/-} myeloid cells spontaneously expand in a normal environment, in response to presumably normal extracellular signals. These data suggest that A20^{-/-} myeloid cells exhibit cell-autonomous defects that drive their differentiation, proliferation and/or activation.

### H. Normal Myeloid Commitment of A20^{-/-} Stem Cells, but Increased Proliferation of Committed A20^{-/-} Myeloid Progenitors in Bone Marrows

To examine whether the differentiation of A20^{-/-} fetal liver stem cells is abnormal, i.e., whether developmental signals to myeloid cells are regulated by A20, the inventors measured the number of BrdU⁺ Gr-1⁺ Mac-1⁺ cells in bone marrows from A20^{-/-} and A20^{+/+} mice that had been injected with BrdU three hours prior to sacrifice. These analyses revealed approximately twice the number of BrdU⁺ myeloid progenitors in A20^{-/-} than in A20^{+/+} bone marrows, indicating that increased proliferation of these committed myeloid progenitors occurs in these mice (FIG. 31). To determine if increased numbers of progenitors might result from increased differentiation of stem cells to the myeloid lineage, the inventors examined the number of primitive myeloid progenitors by performing CFU-GM assays. In these studies, 15×10³ bone marrow cells from A20^{+/+} or A20^{-/-} mice are cultured with IL-3, IL-6 and c-kit in methylcellulose for 10 days, after which CFU-GM colonies are characterized as “large” (>50 cells) or “small” (<50) colonies and counted. Preliminary data from these studies revealed that A20^{-/-} bone marrow cells contain comparable numbers of CFU-GMs as A20^{+/+} bone marrow cells. This finding suggests that primary myeloid commitment is normal in the absence of A20, and the increased proliferation of myeloid progenitors in bone marrow largely represents aberrant responses of committed myeloid progenitors.

### I. A20^{-/-} Mice Die Acutely in Response to Low, Single Doses of LPS or TNF

In addition to this apparent role for A20 in regulating myeloid cell development, the expansion of mature myeloid cells in A20^{-/-} mice may also result from increased activation and homing responses of mature cells to endogenous microbial flora and innate immune stimuli. Thus, the inventors are examining the response of A20^{-/-} myeloid cells to stereotypical innate immune stimuli in vivo.

Bacterial lipopolysaccharide (LPS) and TNF stimulate innate immune cells, including macrophages, and cause dose dependent responses in mice ranging from self-limited inflammation to an acute septic shock syndrome associated with macrophage activation and vascular collapse. TNFR1 is required for the responses to both LPS and TNF. As A20 regulates TNF responses, and as macrophages likely mediate these responses, the inventors examined the response of A20^{-/-} mice to low doses of LPS and TNF. Both LPS and TNF cause A20^{-/-} mice to die within two hours, while A20^{+/+} littermates survive without gross effects (Table 5), indicating that A20^{-/-} mice are indeed hypersensitive to TNF. Although the pathophysiology of this phenomenon may be due to hyperresponsiveness of lymphoid, myeloid, stromal and/or endothelial cells, these findings provide physiological evidence for a critical role for A20 in regulating TNF responses in vivo, and suggest that myeloid cell responses may be aberrant.

Table 5. Increased susceptibility of A20^{-/-} mice to TNF. Survival of A20^{+/+} and A20^{-/-} mice two hours after intraperitoneal injection with the indicated doses of TNF. (ND=not done)

TNF

| | 0.1 mg/kg | 0.2 mg/kg | 0.4 mg/kg |
|---|---|---|---|
| +/+ | 5/5 | 2/2 | 2/2 |
| −/− | 0/4 | ND | ND |

### J. Exaggerated in vivo Responses of A20^{-/-} Macrophages and Granulocytes to Innate Immune Stimuli

To better understand in vivo responses of A20^{-/-} macrophages to innate immune stimuli, the less potent stimulus: thioglycollate was used. Intraperitoneal inoculation with this agent induces recruitment of macrophages to the peritoneal cavity without causing the same degree of activation induced by TNF or LPS. The number of intraperitoneal macrophages in A20^{+/+} and A20^{-/-} mice was examined before and after treatment with thioglycollate. Extensive intraperitoneal lavage of untreated A20^{+/+} and A20^{-/-} mice yields comparable numbers of Mac-1⁺ Gr-1^{Int} macrophages. By contrast, dramatically increased (10-fold) numbers of macrophages are obtained from A20^{-/-} mice compared with A20^{+/+} mice three to five days after intraperitoneal thioglycollate injection. Importantly, no significant morbidity or mortality was observed in A20^{-/-} mice after thioglycollate injection. Thus, physiological recruitment of A20^{-/-} macrophages is markedly enhanced in vivo.

To better understand in vivo responses of A20^{-/-} granulocytes to innate immune stimuli, intraperitoneal casein, an agent known to recruit granulocytes, is being used. Injection of casein into mice 18 hours and 4 hours prior to harvesting peritoneal fluid results in significantly elevated (five-fold) numbers of intraperitoneal Mac-1⁺ Gr-1^{Hi} granulocytes from A20^{-/-} mice as compared with A20^{+/+} mice. Again, no significant morbidity or mortality occurs with casein treatment of A20^{-/-} mice. Thus, physiological granulocyte recruitment is also regulated by A20.

Taken together, the data indicates that A20 is critical for regulating the homeostasis and function of both macrophages and granulocytes. A20's roles in regulating innate immune cells appear to be cell-autonomous. As NF-κB and/or JNK signaling pathways are critical for regulating cellular activation and proliferation responses in most cells—including innate immune cells—and as A20 may regulate NF-κB and JNK responses to TNF, the mechanisms by which A20 regulates these signaling pathways is being investigated.

### Example 15

### A20 is Essential for Terminating TNF Induced NF-κB Responses

Heterologous A20 expression can inhibit TNF induced NF-κB activation in cell lines. NF-κB activates multiple cellular activation genes, including pro-inflammatory genes in immune cells. Thus, if A20 is essential for restricting NF-κB responses, then the severe inflammation seen in A20^{-/-} mice may be partly due to unchecked NF-κB driven inflammatory responses to TNF. To investigate the role of A20 in regulating NF-κB responses, these responses were analyzed in two homogeneous cell populations: (i) thymocytes, which constitutively express A20 mRNA; and (ii) MEFs, which induce A20 mRNA after exposure to TNF. Similar findings were observed in both cell types. NF-κB activity, assessed by electrophoretic mobility shift assay (EMSA), revealed several important findings. First, A20^{-/-} cells display no spontaneous NF-κB activity at rest (FIG. 18). Thus, unlike IκBα, A20 is not essential for the basal repression of NF-κB activity (Beg et al., 1995). As NF-κB is activated by stimuli such as TNF, A20^{+/+} and A20^{-/-} cells were treated with TNF and analyzed for NF-κB activity. These studies revealed induction of NF-κB DNA binding activity approximately 10 minutes after TNF treatment, followed by termination of this activity by 60 minutes in normal cells (FIG. 11). Termination of NF-κB DNA binding activity in normal MEFs occurred between 30 and 60 minutes despite repeated treatment with fresh TNF. By contrast, NF-κB DNA binding activity persisted in A20^{-/-} MEFs (FIG. 18). Thus, the data indicates A20 is essential for terminating TNF induced NF-κB activity.

While the mechanisms by which NF-κB DNA binding activity is normally terminated are incompletely understood, prior work has indicated that IκBα mRNA is transcriptionally activated by NF-κB (Beg et al., 1995). IκBα protein is then synthesized, translocates to the nucleus, binds to and inactivates NF-κB, and relocates to the cytoplasm still bound to NF-κB. It was thus investigated whether the transcription and translation of IκBα in TNF treated A20^{+/+} and A20^{-/-} cells. Northern analyses revealed that IκBα mRNA is readily transcribed in both A20^{+/+} and A20^{-/-} cells within 30 minutes of TNF treatment (FIG. 21). By contrast, Western analyses indicate that IκBα protein, which is phosphorylated and degraded within 10 minutes after TNF treatment, re-accumulates in A20^{+/+} MEFs but not A20^{-/-} MEFs (FIG. 12B).

This finding indicates that A20 may either regulate translation of IκBα mRNA, or prevent degradation of newly synthesized IκBα protein. It is contemplated that A20 performs the latter function by inhibiting the phosphorylation activity of the enzyme complex inhibitor of kappa kinase (IKK), or by regulating a more proximate activation step between TNFR and IKK. To distinguish between these functions for A20, MEFs were treated with the proteosome inhibitor MG-132 fifteen minutes after TNF treatment. This dual treatment led to the reaccumulation of IκBα protein in A20^{-/-} MEFs, indicating that IκBα protein is synthesized normally in the absence of A20, but is rapidly degraded by proteosome dependent pathways (FIG. 22A). As the rapid degradation of IκBα protein could be due to rapid phosphorylation of IκBα by IKK, an examination was done of whether IKK activity in TNF treated MEFs by immunoprecipitating the IKK complex with an anti-IKKγ antibody and then measuring the capacity of this complex to phosphorylate a recombinant GST-IκBα (residue #1-54) substrate. This kinase assay demonstrates that IKK activity is indeed prolonged in A20^{-/-} MEFs, compared to normal cells (FIG. 22B). Thus A20, itself induced by TNF, terminates TNF induced NF-κB signals by inhibiting IKK phosphorylation of IκBα. A20 is essential for this function—even in the presence of de novo synthesized IκBα protein—and is thus is indicated to be a critical regulator of inflammatory gene expression in immune cells.

### Example 16

### A20 may be Required for Terminating TNF Induced JNK Rresponses

Like NF-κB signals, JNK signals are thought to be critical for the regulation of innate immune cell proliferation. The following data suggests that A20 plays a novel and critical role in JNK signaling. A20's regulation of JNK signaling may be independent of its roles in terminating NF-κB signaling, since prolonged JNK signals have previously been associated with NF-κB deficient cells, rather than cells expressing persistent NF-κB activity.

While many NF-κB dependent genes are associated with cellular activation, JNK pathway signaling is important for cellular proliferation responses (Chen et al., 2000). Hence, TNF activation of both NF-κB and JNK pathways may stimulate and coordinate cellular activation and proliferation of immune cells. The ability of heterologous A20 to inhibit TNF induced JNK responses suggests that endogenous A20 regulates JNK activity. Proper regulation of c-Jun activity may be critical in immune cells, since loss of the Jun inhibitor JunB in granulocytes leads to granulocyte expansion and granulocytic leukemia (Tang et al., 2001).

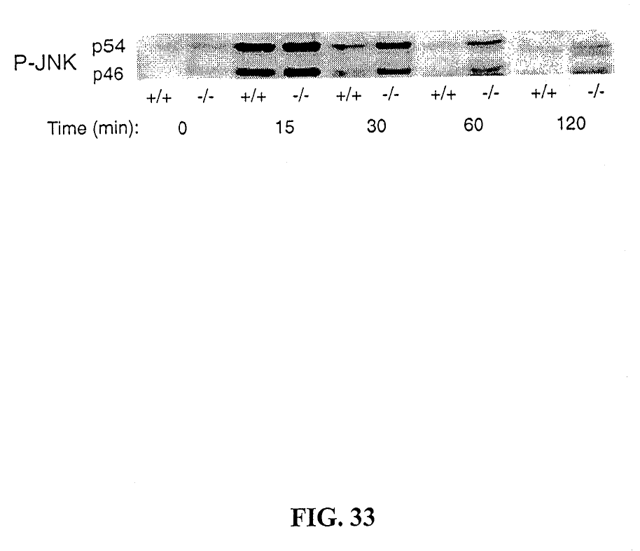

To investigate the role of A20 in regulating TNF induced JNK, the activity of JNK pathway signaling molecules was examined in TNF stimulated thymocytes and MEFs. Similar findings were obtained with both cell types. As in studies identifying NF-κB responses, thymocytes were harvested from young mice to obtain comparable populations of cells. After confirming that comparable populations of CD4⁺ CD8⁺ double positive (DP) thymocytes were obtained from relatively healthy, young mice, cells from these thymi were used for studying TNF induced INK responses. The studies indicate that levels of phosphorylated JNK/SAPK, the kinase that phosphorylates c-Jun, are prolonged in A20^{-/-} compared with A20^{+/+} thymocytes after TNF treatment (FIG. 33). Thus, suggesting that A20 is essential for terminating TNF induced JNK signaling proximate to the level of JNK/SAPK activity.

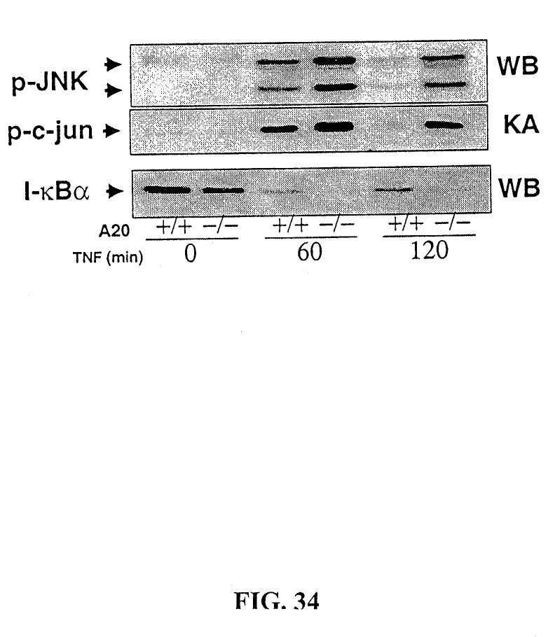

P-JNK phosphorylates c-jun, and phosphorylated c-jun, or P-jun, dimerizes with Fos and other Fos family members to form active transcription factors such as AP-1. Thus, one predicted consequence of aberrantly persistent P-JNK is that A20^{-/-} cells will persistently phosphorylate a c-jun substrate. This prediction was tested by a c-jun kinase assay, wherein cell lysates from TNF treated A20^{+/+} and A20^{-/-} cells were assayed for their ability to phosphorylate a heterologous c-jun substrate. This assay revealed that A20^{-/-} cells indeed display persistent c-jun kinase activity in response to TNF (FIG. 34).

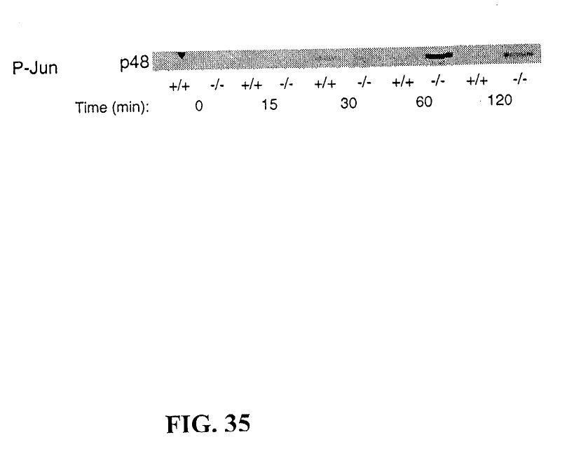

Persistent c-jun phosphorylation would be predicted to yield elevated levels of P-jun in A20^{-/-} cells, and these were indeed identified by Western blotting analysis for P-jun (FIG. 35).

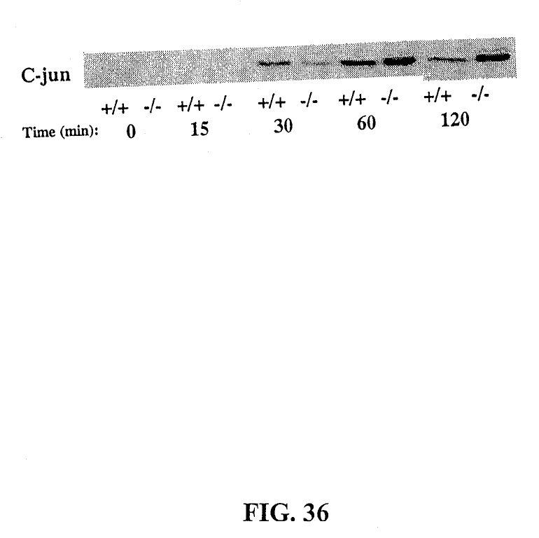

In addition to dimerizing with Fos to form the AP-1 transcription factor complex, P-jun can also dimerize with ATF-2 to form a distinct transcription factor complex. This complex regulates the transcription of c-jun itself Thus, an additional predicted consequence of persistent P-jun activity is that levels of c-jun protein would be elevated. This prediction is supported by direct Western analyses of lysates (FIG. 36).

Taken together, these data indicate that A20 is essential for terminating JNK responses to TNF. This remarkable finding is novel for two reasons. First, this may represent the first identification of a molecule required for specifically terminating JNK signaling. Secondly, while recent studies have suggested that NF-kB pathway signals are important for terminating JNK signals, A20^{-/-} cells surprisingly display both persistent NF-kB and persistent JNK signaling.

### Example 17

### Determining Whether A20^{-/-} RAG-1^{-/-} Double Mutant Mice Spontaneously Develop Inflammation Comparably with A20^{-/-} Mice

TNF stimulates innate immune cells during inflammatory responses and A20 appears to restrict cellular responses to TNF. In addition, characterizations of A20^{-/-} mice indicate that they develop spontaneous inflammation, suggesting that A20 is essential for inhibiting inflammation in vivo. In an effort to understand how A20 performs this essential function, one can focus on the role of A20 in regulating innate immune cells. The rationale for this focus derives from the prominent contribution that myeloid cells make to inflammatory infiltrates in A20^{-/-} mice (FIG. 27). It was also found that A20 protein is expressed in myeloid immune cells. To better define the role of myeloid cells in mediating autoimmunity in A20^{-/-} mice, one can interbreed A20^{-/-} with RAG-1^{-/-} mice. Importantly, data suggests that A20^{-/-} RAG-1^{-/-} double mutant mice spontaneously develop inflammation comparably with A20^{-/-} RAG-1^{+/+} littermates. Thus one can characterize the cellular and molecular nature of inflammatory infiltrates from tissues of A20^{-/-} RAG-1^{-/-} and A2^{-/-} RAG-1^{+/+} mice by quantitating subsets of myeloid cells (Mac-1⁺ GR-1^{Int} F480⁺ macrophages, Mac-1⁺ CD11c⁺ F480⁻ dendritic cells, and Mac-1⁺ GR-1^{Hi} granulocytes) by flow cytometry and immunohistochemistry, assess cytokine (e.g., IFNγ, TNF, IL-12) production by flow cytometry of extracted cells and perform RPA of mRNA from tissues (TNF, IFNγ, MIP-1α) from these mice. As the presence of increased numbers of immune cells in non-lymphoid tissues is a sign of immune cell activation and differentiation that suggests that these cells are displaying effector function and causing inflammatory damage, one may wish to evaluate the number and activation state of immune cells in non-lymphoid tissues such as the liver, intestine and lungs, as well as in lymphoid tissues (spleen, lymph nodes). These studies will demonstrate the capacity of A20^{-/-} myeloid cells to cause spontaneous inflammation in the absence of T lymphocytes. Furthermore, A20^{-/-} RAG-1^{-/-} mice will be a useful model for further studying the mechanisms by which A20 regulates innate immune cells in vivo. Interpretation of the results will require the cellular and genetic restriction of A20 deficiency to defined subsets, as described below.

### Example 18

### Determining Whether Chimeric Mice Reconstituted with Fetal Liver Hematopoietic Cells from Either A20^{-/-} or A20^{-/-} RAG1^{-/-} Double Mutant Mice Develop Inflammation

Observations that A20^{-/-} RAG-1^{-/-} mice develop severe inflammation indicate that A20^{-/-} myeloid cells and/or A20^{-/-} stromal cells play major roles in mediating this inflammation. To determine the extent to which A20^{-/-} myeloid cells cause inflammation in the presence of normal stromal and endothelial cells, fetal liver cells containing hematopoietic stem cells will be transferred from A20^{±} or A20^{-/-} E15.5 embryos into lethally irradiated C57B1/6J mice bearing the Ly5.2 hematopoietic surface marker. A rapid DNA preparation and PCR strategy can be used to allow genotyping of embryos within six hours of embryo harvest. Fetal liver stem cells from appropriate embryos are then transferred intravenously. Donor cells from a C57B1/6J/129 background are Ly5.1⁺, allowing confirmation that recipient hematopoietic cells (Ly5.2⁺) have been eliminated by irradiation, as well as facilitating analysis of donor cells. After donor stem cells have reconstituted the chimera over a period of 8-16 weeks, tissues from these mice are studied to determine whether and to what degree inflammation occurs. The nature and severity of inflammation in these mice are assessed by flow cytometric, histological and molecular studies described in herein. Studies of this nature indicate that A20^{-/-} hematopoietic cells indeed accumulate and cause inflammation in chimeric mice (FIG. 30). This expansion of donor myeloid cells occurs in the face of consistently minimal reconstitution of donor T and B lymphocytes. Thus, these findings suggest that A20^{-/-} myeloid cells possess cell-intrinsic defects that support their spontaneous accumulation in vivo. However, cross-talk between A20^{-/-} myeloid cells and the few A20^{-/-} T cells can not be ruled out or precisely measured in these chimeric mice.

To determine unequivocally the capacity of A20^{-/-} myeloid cells to expand in the absence of lymphoid or stromal A20^{-/-} cells, fetal liver stem cells are transferred from A20^{-/-} RAG-1^{-/-} and A20^{±} RAG-1^{-/-} embryos into lethally irradiated Ly5.2⁺ mice. The only A20^{-/-} cells in the resulting chimeric mice should be myeloid (or NK) cells. Thus, analyses of these mice should reveal largely cell autonomous defects in A20^{-/-} myeloid cells. Studies of this nature indicate that both macrophages and granulocytes accumulate in greater numbers in chimera reconstituted with A20^{-/-} RAG-1^{-/-} than with A20^{±} RAG-1^{-/-} stem cells (FIG. 30). As these cells generally respond rapidly to the presence of pathogen associated microbial motifs via Toll like receptors, as well as associated innate immune stimuli (e.g., IFNs, TNF), it is possible that A20^{-/-} myeloid cells respond excessively to stimuli elicited by endogenous microbial flora (e.g., intestinal bacteria).

Granulocytes also respond to factors secreted by macrophages (e.g., TNF). Thus, it the aberrant granulocyte function may occur secondary to aberrant macrophage function. While macrophage deficient (e.g., MCSF deficient) and granulocyte deficient (e.g., GCSF deficient) mice are incompletely deficient for these cell types and also exhibit confounding phenotypes (i.e., bone marrow failure due to osteopetrosis in MCSF^{-/-} mice), and are thus less useful for genetic dissection studies than lymphocyte deficient (e.g., RAG-1^{-/-} and RAG-2^{-/-}) mice, this issue is addressed by co-transferring Ly5.1⁺/Ly5.1⁺ A20^{±} and Ly5.1⁺/Ly5.2⁺ A20^{-/-} fetal liver stem cells into lethally irradiated Ly5.2⁺/Ly5.2⁺ mice. This mixed chimera study will allow direct comparison of the homeostasis of A20^{±} versus A20^{-/-} cells within the same mouse. If A20^{-/-} macrophages accumulate to a greater degree than A20^{±} macrophages (as predicted), but A20^{-/-} and A20^{±} granulocytes expand comparably, then A20^{-/-} macrophages likely secrete factors that account for abnormal granulocyte function in vivo. As these chimeric studies may also yield less clear results, an alternative approach will be taken in vivo and in vitro to assess the specific responses of purified populations of these and other myeloid lineages (e.g., dendritic cells), as is described herein below.

All of the technical aspects of fetal liver hematopoietic stem cell transfer have already been established, including fetal liver harvest, rapid PCR genotyping, and successful maintenance and reconstitution of lethally irradiated chimera. The pitfalls in interpretation of these studies are lessened by the use of chimeric mice where A20 deficiency is localized to myeloid cells. However, further definition of the cellular defects of specific myeloid lineages will require complementary in vivo and in vitro studies where specific cell types are purified and stimulated (see below). For example, macrophages can be purified will be purified from bone marrow cultures and thioglycollate treated mice, while granulocytes will be purified from casein treated mice. Ultimately, these studies will be further enhanced by development of mutant mice bearing lineage specific deletions of the A20 gene.

### Example 19

### Examining the Homeostasis of A20^{-/-} Innate Immune Cells

The accumulation of myeloid immune cells in A20^{-/-} and A20^{-/-} RAG-1^{-/-} mice, as well as chimeric mice reconstituted with stem cells from these mice indicates that the homeostatic regulation of A20^{-/-} myeloid cells is aberrant. As myeloid cells typically divide as committed precursors in the bone marrow and then migrate to peripheral tissues after terminally differentiating, increased numbers of these cells may reflect increased proliferation of precursors or excessive persistence of mature cells. To determine the rate at which myeloid cells in bone marrows or peripheral tissues are proliferating, BrdU is injected into mice followed by combining cell surface with intranuclear staining to quantitate BrdU incorporation into cells of these lineages. Innate immune cells are studied from lymphoid and non-lymphoid tissues at various time points after BrdU treatment to gain insight into which compartments (e.g., bone marrow, spleen, or non-lymphoid tissues) harbor proliferating cells. Studies suggest that markedly increased BrdU incorporation is present in Mac-1⁺ Gr-1^{Int} F480⁺ macrophages and Mac-1⁺ Gr-1^{Hi} granulocytes from A20^{-/-} mice from multiple tissues after a short term BrdU pulse (three hours) (FIG. 31). This data indicates a major role for increased cell cycling rather than increased cell survival of these cells in causing their progressive accumulation. While increased survival may also contribute, changes in cell survival will be difficult to assess in the presence of dramatically increased proliferation. This is particularly true in vivo, where apoptotic cells are rapidly cleared. Thus, changes in cell survival may be best assessed in vitro (see below). These studies will be performed initially in A20^{-/-} and A20^{-/-} RAG-1^{-/-} mice. If aberrant numbers of these cells are confirmed in these mice, then the studies will be conducted in chimeric mice bearing stem cells from these mice, as described herein.

Increased proliferation of myeloid cells may result from the presence of excessive growth factors, or aberrant responses of the myeloid cells, or both. Hence, the levels of cytokines known to stimulate proliferation and homing of macrophages (e.g., MCSF, TNF) and granulocytes (e.g., GM-CSF, G-CSF, TNF, KC, MIP-1α) will be examined by measuring mRNA for these factors by RPA from tissues of mice. Preliminary analyses reveal that tissue levels of TNF are markedly elevated in intact A20^{-/-} and A20^{-/-} RAG-1^{-/-} mice (FIG. 29). Thus, these studies are both feasible and likely to be revealing. They will be confirmed and extended to chimeric mice. To determine whether these populations respond aberrantly to standardized stimuli in vivo, the responsiveness of A20^{-/-} mice to intraperitoneal casein or thioglycollate is studied. Casein elicits granulocyte migration after two injections four and eighteen hours prior to harvesting tissue; and thioglycollate elicits macrophage migration three to five days after injection. Preliminary experience with these agents reveals that dramatically elevated (i.e., ten fold greater) numbers of these cells are obtained from A20^{-/-} mice as compared with A20^{±} mice, suggesting that in vivo responses of A20^{-/-} innate immune cells are markedly exaggerated. These studies will provide information as to the levels of stimulatory growth factors as well as the physiological responsiveness of A20^{-/-} innate immune cells in vivo.

In vivo responses to agents such as thioglycollate can reflect several acute innate immune stimuli involving more than one cell type. Thus, to determine whether the direct responses of A20^{-/-} innate immune cells to standardized amounts of growth factors are abnormal, the responsiveness of purified populations of these cells to standardized amounts of growth factors will be measured in vitro. Macrophages are purified either from young mouse spleens by FACSorting, or from bone marrow derived cultures (elicited by growth in 25% L929 cell line conditioned media). Dendritic cells are also derived from either spleens or bone marrow cultures grown in GM-CSF and IL-4. Proliferation (and survival) of these cells will then be assayed in vitro in response to standardized amounts of growth factors. Granulocytes will be purified by FACSorting from spleens and similarly assayed for responsiveness to GM-CSF. In vitro studies will also allow more ready assessment of proliferative versus survival affects upon cell numbers, as apoptotic cells will be more easily quantitated.

Most proliferating myeloid cells in vivo are immature. Indeed, preliminary data suggests that marked increases in the number of BrdU⁺ macrophage lineage cells are seen in the bone marrow as well as in the spleen and liver (FIG. 31). These increased proliferating bone marrow cells could reflect increased proliferation responses of committed myeloid precursors or could also reflect aberrant differentiation of stem cells into myeloid lineage rather than other hematopoietic cells. Indeed, the biased reconstitution of lethally irradiated mice with A20^{-/-} myeloid cells rather than A20^{-/-} T cells provides some support for this idea. Thus, the number of colony forming units of granulocyte-macrophage lineage (CFU-GM) will be quantified in spleens and bone marrows of A20^{+/+} and A20^{-/-} mice. These assays are performed with cytokine impregnated methylcellulose cultures, and preliminary studies suggest that A20^{-/-} mice possess comparable numbers of bone marrow derived CFU-GM. Thus, A20 may regulate the proliferation of committed myeloid progenitors and not the commitment toward myeloid lineage stem cells. BrdU studies will also be extended from developing A20^{-/-} cells to chimeric mice bearing A20^{-/-} stem cells. These studies are particularly important since the differentiation of IκBα^{-/-} lymphoid precursors is compromised in intact IκBα^{-/-} mice, but relatively normal when transferred into normal hosts. Taken together, the studies will further reveal how A20 regulates immune cell homeostasis.

As noted, it was found that LPS and TNF cause acute morbidity and mortality in A20^{-/-} mice (Table 5). However, this toxicity is not observed with thioglycollate or casein treatment. Thus, in vivo studies with these latter innate immune stimuli will be feasible. Given the pleiotropic expression of A20 in multiple cell types, it is very possible that multiple functions for A20 will be found in either directly or indirectly regulating innate immune cell function in vivo. Thus, it is believed that the proposed combination of in vitro and in vivo studies should yield significant insights into these functions. Further studies examining the expression of A20 mRNA and protein (in both resting and stimulated cells) and functional studies of defined populations of A20^{-/-} innate immune cells (below) will further clarify A20's functions in these cells.

### Example 20

### Studying Functional Responses of A20^{-/-} Innate Immune Cells

### A. In vivo Responses of Innate Immune Cells

To assess the role of A20 in regulating functional responses of innate immune cells in vivo, the expression of molecules associated with the respective effector functions of these cell types is examined. Similar techniques (e.g., flow cytometry, RPA, ELISA) will be used for studying the homeostasis of these molecules to examine the functional status of these cells. The expression of molecules such as iNOS, IFNγ, TNF, IL-1 and IL-12 will be examined in tissues and cells from intact A20^{-/-} mice as well as chimera bearing A20^{-/-} hematopoietic cells. Studies evaluating A20's role in macrophage function suggest that TNF expression (probably derived from macrophages) is elevated in tissues from both intact and chimeric mice (FIG. 29). Furthermore, as the accumulation of proliferating A20^{-/-} innate immune cells in tissues could be due to perturbed differentiation of myeloid precursors which aberrantly migrate to peripheral tissues, these studies may allow confirmation that myeloid cells in peripheral tissues are in fact mature cells responding aberrantly to innate immune stimuli.

In addition to evaluating innate cell function by ex vivo analyses of A20^{-/-} cells and tissues, stimuli known to elicit innate immune cell responses will be used, including LPS, TNF, thioglycollate and casein. Preliminary studies indicate that low doses of LPS and TNF may be lethal to intact A20^{-/-} mice. This may be related to A20's roles in macrophages and/or endothelial cells. These studies thus will be repeated with chimeric mice reconstituted with A20^{-/-} hematopoietic cells. If the mice die, then it will be established that macrophages (rather than endothelial cells) play a key role in this hypersensitivity to LPS. If the mice are resistant to LPS, then a critical role for A20 in endothelial cells is suggested. In the latter scenario, A20^{-/-} macrophages from these mice could then be studied ex vivo. The functional status of macrophages and granulocytes harvested from thioglycollate or casein treated mice will also be assesed, as described above. Studies of A20^{-/-} cells that have been stimulated will be particularly important since A20 expression is induced in multiple cell types (FIG. 12).

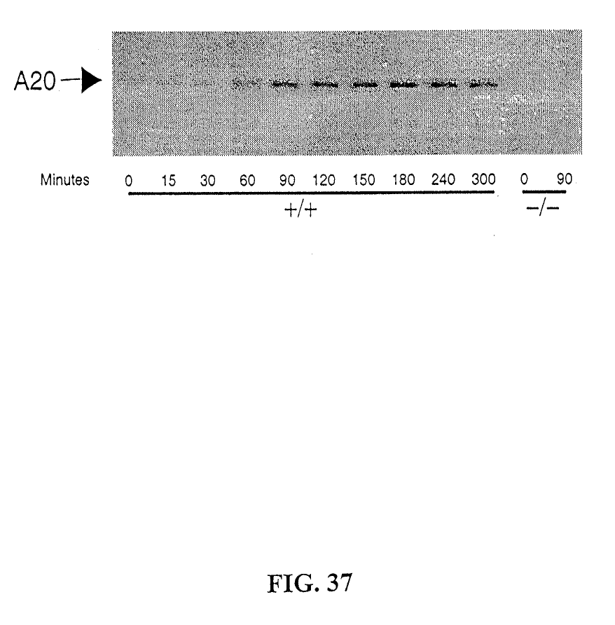

Another myeloid cell population which is particularly important to the integration of innate and adaptive immune responses is dendritic cells. These cells stimulate T lymphocytes during the MHC dependent presentation of antigen via cell surface bound (e.g., CD40, B7) and soluble (e.g., IL-12, IL-15) factors, and may stimulate T cells in the absence of antigen as well. As with functional responses of other innate immune cells, the expression of many of these proteins is dependent upon NF-κB signaling. Thus, they may be regulated by A20. The in vivo functions of A20^{-/-} dendritic cells will be assessed by growing these cells from bone marrow cultures (GM-CSF plus IL-4), loading them with a peptide derived from ovalbumin protein, SIINFEKL, and transferring them into syngeneic C57B1/6J mice bearing 2×10⁶ adoptively transferred CD8⁺ OT-1⁺ TCR transgenic T cells. This procedure was used to induce T cell responses in normal mice with normal dendritic cells (FIGS. 37B, C).

To perform these studies with A20^{-/-} dendritic cells, A20^{±} mice are being backcrossed to a C57B1/6J background to allow adoptive transfer of mature A20^{-/-} dendritic cells into non-irradiated mice. These mice have been bred for five generations to C57B1/6J mice thus far, and will be studied after they are eight generations backcrossed. These studies will assess in vivo functional capacity of A20^{-/-} dendritic cells. Other aspects of dendritic cell function will be examined in vitro (see below).

### B. In vitro Responses of Purified A20^{-/-} Innate Immune Cells

The in vivo responses of A20^{-/-} innate immune cells will provide important and physiological clues to A20's functions. To gain more defined information about A20's functions in specific cell types, the in vitro responses of purified innate immune cell types will be examined. As described above, procedures were established for isolating purified populations of these cells directly from spleens ex vivo, or from bone marrow cultures. Thus, purified populations of macrophages, granulocytes and dendritic cells are first being tested to document that A20 is expressed in these cell types. Importantly, preliminary data suggests that A20 is indeed expressed in these cells. Thus, A20 may directly regulate the function of these cells. Purification of these cell types for functional studies is facilitated by use of A20^{-/-} RAG-1^{-/-} and RAG-1^{-/-} mice as sources of cells for these studies. Preliminary observations that similar autoimmune inflammation occurs in A20^{-/-} and A20^{-/-} RAG-1^{-/-} mice supports the idea that innate immune cells isolated from A20^{-/-} RAG-1^{-/-} mice will recapitulate defects seen in A20^{-/-} mice.

To better understand the role of A20 in directly regulating macrophage function, macrophages that are purified from thioglycollate injected mice will be studied as well as those derived from bone marrow cultures supplemented with M-CSF. Both sources of macrophages will be used since thioglycollate derived macrophages are likely derived from circulating monocytes and may thus represent more mature cells than bone marrow cells grown in macrophage colony stimulating factor (M-CSF) supplemented media. Macrophages are then purified either by plate adherence or FACS sorting, depending on the purity and number of cells required. The expression of NF-κB dependent genes will the be assayed. Thus, TNF treated purified macrophages will be assayed at various time points (0, 3, 6 and 12 hours) for expression of B7 (CD86), CD40 and MHC Ia surface markers, as well as intracellular and secreted IL-6, IL-1, GM-CSF, MIP1α and IFNγ proteins. Finally, as preliminary data indicates that A20 is required for terminating NF-κB responses in MEFs, the numbers of cells will be increased and nuclear lysates harvested from these cells to directly examine whether NF-κB responses are similarly prolonged in innate immune cells by EMSA.

Similarly, dendritic cell function will be investigated in vitro by culturing bone marrow cells in the presence of GM-CSF and IL-4, and then studying the maturation response (i.e., expression of surface markers and phagocytic function) of these cells to LPS or poly-inosine/cytosine, agents known to activate dendritic cells by stimulating Toll like receptors. The NF-κB response to TNF will be studied before and after maturation. These studies will also be performed on dendritic cells purified from spleens, as splenic dendritic cells may include subsets that are distinct from bone marrow derived dendritic cells.

To study the responses of purified granulocytes, granulocytes will be used that are elicited either by intraperitoneal casein or by two to three day bone marrow cultures grown in GM-CSF supplemented media. Such granulocytes are purified by gradient centrifugation. Granulocytes purified in this fashion can be further stimulated in vitro with agents such as LPS, TNF or GM-CSF to activate NF-κB and to secrete cytokines such as TNF, IFNα, G-CSF, M-CSF, IL-8, IL-6, and IL-1. These studies will determine the role(s) of A20 in directly regulating granulocyte function. Taken together, these studies should draw together biochemical and cellular understanding of the role(s) of A20 in regulating the function of these innate immune cells. Biochemical studies of these cells will require pooling of cells from multiple mice to obtain sufficient cell numbers. Alternative approaches that may yield additional insights into A20's functions in individual cell types will include the creation of lineage specific gene targeting of A20 using lox-cre technology, and such studies are currently underway.

### Example 21

### Determining the Mechanism(s) by Which A20 Regulates TNF Induced NF-κB Activity

TNF stimulates the transcription of multiple genes involved in cellular activation and proliferation in immune cells via the activation of NF-κB signaling pathways and, as indicated herein, JNK pathways. The studies described herein indicate that A20 may be essential for terminating TNF induced NF-κB activity. Accordingly, the biochemical mechanism by which A20 performs this critical function will be determined. These studies are being performed in MEFs because the paucity of TNFR molecules on most cells requires that large numbers of uniform cells be used for many biochemical studies, and because preliminary data suggests that similar signaling defects exist in thymocytes and MEFs. As noted above, these biochemical signaling defects in MEFs will be correlated with studies in primary innate immune cells to better understand A20's functions in regulating innate immune cell function.

### Example 22

### Determining A20's Associations with Proximate TNFR Signaling Molecules

To begin to elucidate the mechanism by which A20 regulates TNF induced NF-κB responses, an A20 specific antiserum has been generated to detect the presence of A20 protein as well as its potential interactions with other proteins. Immunization of rabbits with an A20 specific peptide and affinity purification of the resulting antiserum yielded an antiserum which detects an approximately 82 kD band in A20^{+/+} but not A20^{-/-} MEFs (FIG. 38). Co-incubation of the antiserum with the specific peptide used for immunization eliminates this 82 kD band. Thus, this antiserum appears to be specific for murine A20. Preliminary studies with this antiserum suggest that A20 protein is present at basal levels in both thymocytes and MEFs, and that A20 protein levels increase in MEFs after TNF stimulation (FIG. 38).

Preliminary data suggest that A20 is essential for terminating TNF induced NF-κB responses by interrupting NF-κB signaling at or above the level of IKKγ. TNFR signaling molecules that are membrane proximate to IKKγ include TRADD, TRAF1/2, RIP, and MEKK3. TNF induced activation of NF-κB absolutely requires RIP and IKKγ, and also appears to involve TRAF and MEKK3. As proximate signaling molecules including TRADD, TRAFs, RIP and IKKγ associate with TNFR1 in a ligand dependent fashion, and as the recruitment of these molecules may be essential for TNF induced NF-κB activity, it will be important to determine which proteins associate with endogenous A20 and which interactions are important in A20's ability to regulate NF-κB signaling. Prior studies have suggested that heterologous A20 can associate with TRAF1 and TRAF2, as well as IKKγ when over-expressed in cell lines. However, they did not establish whether endogenous A20 protein participates in these associations, or to what degree these associations might be functionally significant in the regulation of NF-κB signaling.

To determine whether endogenous A20 associates with proximate TNFR signaling molecules, the recruitment of these molecules to TNFR1 will be examined. First, it will be examined whether A20 is recruited to TNFR1 in response to TNF. Therefore, 5×10⁷ A20^{+/+} MEFs are being stimulated with TNF and immunoprecipitating lysates with the same anti-TNFR1 antibody used by these investigators. Anti-TNFR1 antibody will first be covalently conjugated to sepharose beads to allow the unequivocal identification of immunoprecipitated proteins (e.g., TRAF2) that co-migrate with immunoglobulin heavy chains. As similar studies have revealed TNF ligand dependent association of TRADD, TRAFs, RIP, IKKγ with TNFR1, these results will be duplicated, and followed by analysis of the same immunoprecipitated lysates for the presence of A20 protein. Then lysates from TNF treated MEFs will be analyzed at 15, 30, 60 and 90 minute time points to determine when A20 is recruited to the TNFR complex. In this way, it will be determined whether A20 associates with some or all of these proteins. It will be determined whether A20's association occurs during initial engagement of TNF with resting cells, or whether A20 is recruited to the TNFR only after a period of time (e.g., 30 minutes) correlating with the termination of NF-κB responses.

As it appears that A20 regulates TNFR signaling only after TNF binding, it is possible that A20 protein may only be detected in association with TNFR complexes at later time points. It is also possible that A20's association with TRAFs or IKKγ may serve to disrupt a complex of TNFR signaling molecules, in which case A20 may not be co-immunoprecipitated with TNFR. For example, if A20-TRAF interactions serve to inhibit TRAF interactions with TRADD or TNFR, then it may be found that A20 co-immunoprecipitated with TRAF2 instead of TNFR. Similarly, if A20 binding to IKKγ prevents RIP-IKKγ interactions, then it may be found that A20 co-immunoprecipitated with IKKγ at time points when RIP-IKKγ interactions cease. Therefore, in addition to performing kinetic studies where anti-TNFR is used to immunoprecipitate A20, anti-RIP, anti-TRAF2 and anti-IKKγ antibodies will be used to determine whether A20 co-precipitates with these molecules. These studies will determine precisely when and which proteins associate with A20 during TNFR signaling.

It is believed that all the reagents that should make the co-immunoprecipitation of TNFR signaling molecules feasible have been identified, including an A20 specific antibody and both primary and immortalized A20^{+/+} and A20^{-/-} MEFs. The generation of an A20 specific antibody allows detection of endogenous A20 protein from modest numbers of cells by Western blotting analysis. It is currently being tested whether anti-A20 antibody can also immunoprecipitate A20. If so, A20 will be immunoprecipitated and it will be investigated whether other TNFR signaling molecules are co-precipitated with A20. If the antibody does not serve this function, different immunogenic peptides of the A20 protein will be selected for use as immunogens, and generate more anti-A20 antibodies. An alternate approach is being taken by generating A20-myc fusion protein constructs and transfecting these into A20^{-/-} MEFs prior to stimulating these cells with TNF. This approach utilizing heterologous A20 expression will allow immunoprecipitation of A20 protein directly, and study associated proteins. A functional murine A20 cDNA has already been subcloned into a CMV promoter driven, myc epitope-tagged expression construct. This construct has also been transliently infected into A20^{-/-} MEFs and the anti-A20 antibody used to document conditions in which transfected A20 protein levels mimic endogenous A20 levels. These studies have two advantages over prior studies in that one can: (i) document endogenous levels of heterologous A20 constructs; and (ii) use this system to complement A20^{-/-} MEFs with normal and mutant forms of A20 to dissect potential functional domains of this protein.

### Example 23

### Examining Interactions Between TNFR Signaling Molecules in A20^{+/+} and A20^{-/-} MEFs

As A20 appears to inhibit NF-κB signaling, and as the recruitment of several TNFR signaling molecules to TNFR1 is thought to be essential for TNFR mediated activation of NF-κB, the potential interaction of A20 with proximate TNFR signaling molecules may displace molecules from these complexes that are essential for NF-κB signaling. Thus, it will be examined whether the presence or absence of physiological A20 affects the kinetics of association of TNFR signaling molecules, and whether it affects downstream NF-κB activity. This approach will complement the studies outlined above by correlating interactions between molecules known to be critical for IKKγ activation (and thus, downstream NF-κB activity) with A20's presence in TNFR signaling complexes. Importantly, these studies should reveal A20's functional effects on proximate TNFR signaling even if it is not possible to directly detect A20's interactions with known signaling molecules. For example, if A20 indirectly disrupts RIP interactions with MEKK3 or IKKγ, then the kinetics of RIP-IKKγ interactions will likely be prolonged in TNF treated A20^{-/-} MEFs as compared to A20^{+/+} MEFs.

To examine the role(s) of A20 in regulating interactions between critical TNFR signaling molecules, similar techniques and reagents will be used as described herein to examine the kinetics of these interactions in TNF treated A20^{-/-} and A20^{+/+} MEFs. RIP recruitment to the TNFR is thought to induce oligomerization of IKKγ molecules, which in turn activate IKKα/IKKβ/IKKγ (“signalosome”) complexes that phosphorylate IκBα. Both RIP and IKKγ are required for TNF induced NF-κB activation. Thus, RIP-IKKγ interactions will be examined by immunoprecipitating IKKγ from lysates of TNF treated A20^{-/-} and A20^{+/+} MEFs, and analyzing these immunoprecipitates by Western blotting for the presence of RIP protein. Lysates will be assayed at 0, 15, 30, 60 and 90 minutes after TNF treatment. Aliquots of all immunoprecipitates will also be assayed for the quantity of IKKβ by western blot analysis to confirm comparable amounts of IKK complex in all samples. The kinetics of RIP-IKKγ interactions will then be compared in lysates from A20^{-/-} and A20^{+/+} MEFs to determine whether physiological A20 regulates the duration of RIP-IKKγ association. As RIP-IKKγ interactions are thought to be essential for TNF induced activation of the IKK “signalosome,” immunoprecipitates of TNF treated MEF lysates will be analyzed for their functional capacity to activate NF-κB activity. Thus, immunoprecipitated lysates will be incubated with a GST-IκBα (aa 1-54) fusion protein and ³²P-ATP, and assayed for IKK kinase activity. Aliquots of these TNF treated MEFs will also be assayed for NF-κB activity by EMSA. Finally, to confirm that A20 directly modulates these interactions, A20^{-/-} MEFs will be complemented with the A20 cDNA expression construct to confirm that abnormalities in these cells are solely due to the lack of A20.

Preliminary data suggest that A20 restricts the duration of IKKγ activity in TNF treated MEFs (FIG. 28). If the duration of RIP-IKKγ interactions is similar in A20^{+/+} and A20 MEFs, then A20 may directly inhibit the activity of IKKγ. This possibility can be directly interrogated by adding purified A20 protein to immunoprecipitates from TNF treated MEFs. This assay may directly reveal a novel function for A20, and allow more precise biochemical studies of how A20 regulates RIP-IKKγ interactions. For example, if it is determined that A20 interacts directly with IKKγ, then A20 may disrupt the IKK signalosome via regulation of its phosphorylation, oligomerization, or protein stability. Alternatively, if the duration of RIP-IKKγ interactions is prolonged in A20^{-/-} MEFs (compared to A20^{+/+} MEFs), then A20 may either disrupt this association directly, or via interactions with more proximate TNFR signaling molecules such as TRAFs. These studies should reveal whether A20 regulates the stability or activity of these ligand dependent signaling complexes.

Importantly, the inventors have already documented the association of endogenous TNFR with TRADD and RIP proteins with an anti-TNFR1 antibody (FIG. 39). Also, RIP has been immunoprecipitated with an anti-IKKγ antibody. As a complementary alternative approach to studying the associations of these proteins, an alternative approach will be used for transfecting epitope tagged heterologous RIP, IKKγ, TRAF2 constructs into A20^{+/+} and A20^{-/-} MEFs. In these studies,

### Example 24

### Examining the Role of A20 in Regulating signals from TNFR2, CD40 and LPS

Multiple receptor signals lead to activation of the NF-κB signaling pathway, and most signaling molecules in the TNF induced NF-κB activation pathway are shared by other receptors. For example, TRAF2 is involved in NF-κB activation by other TNFR family members such as TNFR2 and CD40, as well as LPS and IL-1 signaling. RIP is essential for NF-κB activation by TNF, but does not appear to be involved with LPS or IL-1 signaling, and thus may be utilized by a more limited set of receptors. IKKγ appears to be required for TNF, LPS and IL-1 induced NF-κB signals. Thus, to both gain biochemical insights into the mechanism(s) by which A20 regulates TNF induced signaling, as well as to learn more about the biological roles that A20 may play in vivo, the role of A20 in regulating other stimuli of NF-κB activation will be investigated.

TNF binds to TNFR1 and TNFR2. TNFR2 is thought to resemble TNFR1 in utilizing TRAF2 to signal to IKK and NF-κB. However, unlike TNFR1, TNFR2 does not directly interact with death domain containing proteins such as TRADD, suggesting that initial signaling events resulting from TNFR2 engagement may differ from those following TNF binding to TNFR1. These receptors perform non-redundant functions in vivo, as both TNFR1^{-/-} and TNFR2^{-/-} mice display abnormal phenotypes. In addition, TNFR2 may modulate TNFR1 signaling. To determine if TNFR2 induced signals to IKK are regulated by A20, the kinetics of TNF induced NF-κB activation in A20^{-/-} TNFR1^{-/-} cells will be analyzed by EMSA and IKK kinase assay, as described herein (FIG. 28). To dissect the roles of TNFR1 and TNFR2 mediated NF-κB signals, A20^{-/-} mice are being interbred with TNFR-1^{-/-} mice. If these mice develop spontaneous inflammation, and if cells from these mice exhibit aberrant NF-κB responses to TNF, this finding will provide compelling evidence that A20 regulates signals emanating from TNFR2. If this finding is confirmed, then the mechanism of A20's action will be investigated in TNFR2 dependent signaling by examining which protein(s) (e.g., TRAF2, IKKγ) associated with A20 in cells from TNFR1^{-/-} mice. MEFs have already been derived from TNFR1^{-/-} mice and the inventors will study macrophages and thymocytes from these mice as well, since TNFR2 is constitutively expressed on thymocytes, and inducible on several other cell types. On the other hand, if TNF induced NF-κB signals are normal in A20^{-/-} TNFR-1^{-/-} cells, then A20 may regulate TNFR1 and not TNFR2 induced NF-κB activity.

CD40 is a potent co-stimulatory receptor that stimulates NF-κB signaling in macrophages and other cells when engaged by CD40 ligand (22). Like TNFR1 and TNFR2, CD40 signals may be mediated by the adaptor protein TRAF2. To investigate whether CD40 signals are regulated by A20, the inventors will use macrophages, because these cells are known to express CD40. Splenic macrophages, bone marrow derived macrophages or thioglycollate induced macrophages will be purified. Bone marrow derived macrophages are produced by treating bone marrow cells with M-CSF (or media supplemented with 30% L929 conditioned media) for 8-10 days; and mature macrophages are obtained either by treating mice with intraperitoneal aged thioglycollate for 5 days, or by purifying these cells directly from RAG^{-/-} spleens. These cells are then treated with an agonist anti-CD40 antibody, and NF-κB activity is assayed by IKKγ kinase assay and EMSA, as done for TNF signaling. Western analysis for IκBα levels will also be done to provide complementary data regarding NF-κB signaling activity. If CD40 induced NF-κB activity is terminated normally in A20^{-/-} cells, then A20 probably does not regulate CD40 signals, and may regulate NF-κB signals via interactions other than TRAF2. If CD40 induced NF-κB activity is abnormally prolonged in A20^{-/-} cells, then A20 may regulate CD40 signals in addition to TNF signals (perhaps related to the sharing of the TRAF2 adaptor protein by these receptors). In this case, these studies will be repeated in the presence of neutralizing antibody to TNF. In this way, prolonged NF-κB activity in anti-CD40 treated cells should not be ascribed to secondary release of TNF. Ultimately, A20^{-/-} and TNF^{-/-} mice will be interbred and cells from these mice used to further investigate the roles of A20 in regulating TNF independent signals.

When bound by conserved bacterial motifs, Toll like receptors (TLRs) induce NF-κB activation in one of the most evolutionarily conserved immune signaling pathways. TLR4 is preferentially expressed on innate immune cells and binds LPS. As innate immune cells expand aberrantly in A20^{-/-} mice, and as A20^{-/-} mice appear hypersensitive to LPS, it is possible that A20 is critical for regulating TLR4 signals. Thus, purified A20^{-/-} macrophages and B cells will be stimulated with LPS and NF-κB responses studied in the same ways described above. If NF-κB responses persist abnormally, then these studies will be repeated in A20^{-/-} TNF^{-/-} cells. If A20 is essential for regulating TLR4 responses as well as TNF responses, then A20 may mediate these effects at the shared IKK signalosome complex. Alternatively, A20 may regulate distinct proteins in TNF and TLR4 induced NF-κB signaling pathways. Compiling the data above with the studies described herein will provide significant biochemical and biological insight into A20's functions in vivo.

The studies proposed will be carried out with similar methodology as has been described, so significant difficulties with these assays are not anticipated. The mutant and double mutant cells will be available from mice that are currently being interbred, and the cellular techniques for MEF preparations, macrophage and purification, and LPS and anti-CD40 agonist antibody treatment of cells from these mice have been established.

### Example 25

### Determining Whether A20 Regulates JNK Pathway Signals and Cellular Proliferation

TNFR1 engagement stimulates JNK as well as NF-κB signaling. TNF activates JNK signaling through the recruitment of TRAF2 and downstream members of the MEKK and MAPK family, including MEKK7. Phosphorylation of MAPKs leads to the phosphorylation of SAPK/JNK1 and JNK2, which in turn phosphorylate c-Jun. Phosphorylated c-Jun dimerizes with Fos to create the AP-1 transcription factor that stimulates transcription of c-Jun and other cellular proliferation related genes (e.g., c-myc, cyclin D).

Heterologous A20 can inhibit TNF induced JNK activity in cell lines. The most recent preliminary data suggests that A20^{-/-} cells may exhibit excessive phospho-c-Jun activity. This remarkable result is unlikely to be secondary to persistent NF-κB activity since recent evidence suggests that NF-κB is required for terminating JNK activity (7, 8). Hence, A20 may independently terminate JNK as well as NF-κB responses to TNF.

### Example 26

### Determining Whether Phospho-c-Jun Signaling is Prolonged in A20^{-/-} Cells

Phospho-c-Jun activity can be directly measured by measuring phospho-c-Jun (P-Jun) binding to consensus AP-1 oligonucleotide binding sites in EMSA assays. Thus, to determine whether A20 regulates P-Jun activity in MEFs, A20^{-/-} and A20^{+/+} MEFs will be stimulated with TNF and lysates analyzed at various time points for AP-1 activity by EMSA, coupled with anti-P-Jun antibody supershift assays. Studies indicate that A20^{-/-} cells may exhibit greater levels of P-Jun DNA binding in response to TNF, suggesting that A20 may regulate c-Jun signaling responses to TNF (FIG. 33-35). Moreover, elevated P-JNK and elevated c-Jun levels were found in A20^{-/-} cells in these studies. This finding provides the first evidence of any protein that is essential for terminating JNK signaling. It is even more remarkable when one considers that these A20^{-/-} cells exhibit supranormal levels of NF-κB activity—thought to be required for terminating JNK activity. To elucidate the role(s) of A20 in regulating P-Jun activity, the activity of a variety of JNK pathway signaling proteins will be investigated, including JNK. Phosphorylation of c-Jun is mediated by the phosphorylated form of SAPK/JNK (P-JNK). As the preliminary data suggests that P-Jun activity is higher in A20^{-/-} MEFs, the inventors are measuring P-JNK levels in TNF treated A20^{-/-} and A20^{+/+} MEFs by Western blotting. Preliminary studies reveal that P-JNK levels are higher in A20^{-/-} than in A20^{+/+} MEFs.

c-Jun kinase assays are also being performed on lysates from these cells to confirm that these elevated P-JNK levels actually correlate with elevated capacity to phosphorylate c-Jun. These commercial c-Jun kinase assays utilize recombinant c-Jun bound to beads, which are incubated with lysates and then assayed for the presence of P-Jun. Initial studies with this assay indicate that A20^{-/-} cells possess elevated c-Jun kinase activity. The upstream signaling molecules that lead to SAPK/JNK phosphorylation are less clearly defined. However MEKK7 may be essential for phosphorylation of SAPK/JNK, so increased P-SAPK/JNK activity in A20^{-/-} cells may result from increased MEKK7 activity. Accordingly, TNF treated lysates are assayed for the presence of P-MEKK7. Ultimately, these signaling activities will be correlated with co-immunoprecipitation studies described herein. In particular, the kinetics of A20-TRAF2 interactions will be particularly interesting since TRAF2 is required for TNF induced SAPK/JNK phosphorylation. Taken together, these studies should provide novel insights into the role of A20 in regulating TNF induced c-Jun signaling.

### Example 27

### Determining Whether Excessive Phospho-c-Jun Activity Causes Increased AP-1 Dependent Gene Expression and Hyperproliferation of A20^{-/-} Cells

The activation of c-Jun is associated with the induction of genes that drive cellular proliferation in multiple cell types. Phosphorylation of c-Jun leads to increased levels of c-Jun in a positive feedback loop that involves at least two mechanisms. First, phosphorylation of c-Jun leads to stabilization of c-Jun itself, as it becomes resistant to ubiquitination and degradation. Secondly, c-Jun dimerization with Fos also causes AP-1 dependent transcription of c-Jun. Thus, the levels of c-Jun are one reflection of P-Jun activity. AP-1 also drives the transcription of the c-myc and cyclin D. De novo transcription of these genes leads to increased protein levels that are essential for cellular proliferation in adult cells. Thus, direct assessment of the expression of c-Jun, c-myc and cyclin D proteins in TNF treated A20^{-/-} and A20^{+/+} cells by is carried out by Western analysis. Initial studies reveal that c-Jun levels are indeed increased in A20^{-/-} thymocytes after TNF treatment (FIG. 36). Confirmation and extension of these studies will thus provide important confirmation of the transcriptional consequences of A20's role in regulating P-Jun activity. They may also suggest that proliferation rate is critically regulated by A20.

To determine whether excessive P-Jun activity leads to increased cellular proliferation of A20^{-/-} cells, the proliferation of A20^{-/-} and A20^{+/+} MEFs is investigated in media alone and in response to TNF. Proliferation of MEFs is measured both by simply counting cells after plating similar numbers of cells, as well as by the incorporation of BrdU. The latter technique will be used to complement cell counts because of the possibility that differential cell death may also contribute to different cell yields. Excessive c-Jun activity may lead to increased proliferation of MEFs in a variety of conditions. The use of MEFs in these assays will allow measurement of proliferation in several settings, including basal proliferation rate, rate of initiation of proliferation after quiescence, entrance into quiescence after serum withdrawal, and rate of transversion of cell cycle. Each of these conditions can be assayed by making use of the ability of MEFs to undergo cell cycle arrest in the presence of serum free media (0.1% FCS) and when grown to confluency (contact inhibition).

Basal growth rate is measured by plating replicate wells of the same passage (typically P2 or P3) MEFs at the same confluence and cell number. Cells are then counted every 24 hours after plating. Cells are counted by flow cytometry with propidium iodide staining to assess cell death as well as cell expansion. Initiation of proliferation is measured by first arresting MEFs in 0.1% FCS for 15 hours, after which media containing 10% FCS and BrdU are added to the cells. Cell counts and BrdU incorporation are then measured at 3, 6, 12 and 18 hours after stimulation. Cells from this type of study are also stained with high dose PI (50 ug/ml) to distinguish cells in S phase from G2/M phases, thus allowing measurement of the rate of cell cycle progression. Finally, cells growing in 10% FCS can be cultured in serum free media and then assessed for cell cycle position by PI staining, thus providing an assessment of the ability of cells to exit cell cycle. Cells from these studies will be assayed for the expression of c-myc and cyclin D, and correlated with proliferation rates. If the inventors identify elevated levels of P-Jun in A20^{-/-} MEFs which correlate with increased proliferation, then JNK activity is interfered with directly in these cells using either peptide inhibitors or dominant negative constructs (kindly provided by Dr. A. Lin) to determine if specific interference with JNK signaling (as opposed to NF-κB signaling) reduces these abnormalities.

Ultimately, BrdU proliferation studies and flow cytometric P-Jun determinations are performed in primary innate immune cells to attempt to correlate the findings in MEFs with the hyperproliferative state of A20^{-/-} immune cells. These studies will be facilitated by an antibody that recognizes the native phosphorylated form of c-Jun in primary cells and can thus be detected by flow cytometry (17). This antibody will facilitate analyzing single cells responding to stimuli, and reduce the need for obtaining large numbers of primary immune cells for biochemical studies. If elevated levels of P-Jun are identified in A20^{-/-} immune cells that correlate with increased proliferation, then the inventors will attempt to interfere directly with JNK activity in these cells using JNK1 or JNK2 deficient mice. These mice could be interbred with A20^{-/-} mice to genetically determine the contribution of excessive JNK signaling to the aberrant proliferation seen in A20^{-/-} mice.

The cellular proliferation and cell cycle analyses represent straightforward assays that have been established. The annexin and PI studies (indeed, the sub-G0/G1 peak of PI stains will provide a direct measure of apoptotic cells) will be performroed with proliferation assays to avoid pitfalls where differential rates of programmed cell death (PCD) may contribute to differential yields. In this regard, the observation that A20 is an essential anti-apoptotic molecule argues that its absence is unlikely to cause increased cell yields by decreasing PCD rates. As the role of c-Jun in cellular proliferation is well documented, and a recent study demonstrates the critical role of a direct Jun inhibitor, JunB, in maintaining granulocyte homeostasis in vivo, studies with A20^{-/-} cells will yield insights into the homeostatic maintenance of myeloid cells. Differential functions for JNK signaling in different cell types may prevent generalizing results obtained in MEFs to immune cells.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.

U.S. Pat. No. 5,220,007

U.S. Pat. No. 5,221,605

U.S. Pat. No. 5,238,808

U.S. Pat. No. 5,284,760

U.S. Pat. No. 5,354,670

U.S. Pat. No. 5,366,878

U.S. Pat. No. 5,380,721

U.S. Pat. No. 5,389,514

U.S. Pat. No. 5,635,377

U.S. Pat. No. 5,789,166

U.S. Pat. No. 5,798,208

U.S. Pat. No. 5,830,650

Beg A A, Sha W C, Bronson R T, Baltimore D. 1995. Constitutive NF-kappa B activation, enhanced granulopoiesis, and neonatal lethality in I kappa B alpha-deficient mice. Genes Dev 9:2736-46.

Beg and Baltimore, “An essential role for NF-κB in preventing TNF-alpha-induced cell death,” Science 274:782-4, 1996.

Blumberg, Saubermann, Strober, “Animal models of mucosal inflammation and their relation to human inflammatory bowel disease,” Curr Opin Immunol, 11:648-56, 1999.

Boise, Minn, Noel, June, Accavitti, Lindsten, Thompson, “CD28 costimulation can promote T cell survival by enhancing the expression of Bcl-XL,” Immunity, 3(1):87-98, 1995.

Boone, Dassopoulos, Chai, Chien, Lodolce, Ma, “Differential roles of IL-2 and Fas in regulating lamina propria T lymphocytes. (submitted)

Boone, Dassopoulos, Lodolce, Chai, Trettin, Ma, “IL-2 deficient mice develop colitis in the absence of CD28 costimulation,” (submitted)

Braisted and Wells, “Minimizing a binding domain from protein A,” Proc Natl Acad Sci USA, 93(12):5688-92, 1996.

Brown, He, Westerheide, Boss, “Characterization of the expressed CIITA allele in the class II MHC transcriptional mutant RJ2.2.5,” Immunogenetics,. 43(1-2):88-91, 1996.

Burton and Barbas, “Human antibodies from combinatorial libraries,” Adv Immunol, 57:191-280, 1994.

Cadwell and Joyce, “Randomization of genes by PCR mutagenesis,” PCR Methods Appl, 2(1):28-33, 1992.

Chan, Chun, Zheng, Siegel, Bui, Lenardo, “A domain in TNF receptors that mediates ligand-independent receptor assembly and signaling,” Science, 288;2351-4, 2000.

Chan, Siegel, Lenardo, “Signaling by the TNF receptor superfamily and T cell homeostasis,” Immunity, 13:419-22, 2000.

Chen C L, Singh N, Yull F E, Strayhorn D, Van Kaer L, Kerr L D. 2000. Lymphocytes lacking I kappa B-alpha develop normally, but have selective defects in proliferation and function. J Immunol 165:5418-27.

Cooley, Berg, Spradling, “Controlling P element insertional mutagenesis,” Trends Genet, 4(9):254-8, 1988.

Cooper et al., J. Biol Chem, 271:18068, 1996.

Corazza, Eichenberger, Eugster, Mueller “Nonlymphocyte-derived tumor necrosis factor is required for induction of colitis in recombination activating gene (RAG)-2^{-/-} mice upon transfer of CD4⁺ CD45RB^{Hi} T cells,” J Exp Med, 190:1479-92, 1999.

Dassopoulos T, Boone D L, Lodolce J P, Chai S, Trettin S, Ma A. “Mucosal immune lessons from gene targeting experiments,” in Intestinal Mucosa and its Diseases (Domschke W et al, eds) Kluwer Adad Pub, 1998).

Davidson, Leach, Fort, Thompson-Snipes, Kuhn, Muller, Berg, Rennick, “T helper cell 1-type CD4⁺ T cells, but not B cells, mediate colitis in interleukin 10-deficient mice,” J Exp Med. 84(1):241-51, 1996.

De Smaele E, Zazzeroni F, Papa S, Nguyen D U, Jin R, Jones J, Cong R, Franzoso G. 2001. Induction of gadd45beta by NF-kappaB downregulates pro-apoptotic JNK signalling. Nature 414:308-13.

Egholm et al., Nature 1993, 365, 566

Fiocchi, “From immune activation to gut tissue injury: the pieces of the puzzle are coming together,” Gastroenterology, 117:1238-41, 1999.

Giroir, Brown, Beutler, Proc Natl Acad Sci USA, 89:4864, 1992.

Goeddel D V. 1999. Signal transduction by tumor necrosis factor: the Parker B. Francis Lectureship. Chest 116(1 Suppl):69S-73S.

Heath, Kurts, Caminschi, Carbone, Miller, “CD30 prevents T-cell responses to non-lymphoid tissues,” Immunol Rev, 169:23-9, 1999.

Heyninck et al., J Cell Biol, 145:1471, 1999.

Hilton, Watowich, Katz, Lodish, “Saturation mutagenesis of the WSXWS motif of the erythropoietin receptor,” J Biol Chem, 271(9):4699-708, 1996.

Iizuka, Chaplin, Wang, Wu, Pegg, Yokoyama, Fu, “Requirement for membrane lymphotoxin in natural killer cell development,” Proc Natl Acad Sci USA, 96:6336-40, 1999.

Ina, Itoh, Fukushima, Kusugami, Yamaguchi, Kyokane, Imada, Binion, Musso, West, Dobrea, McCormick, Lapetina, Levine, Ottaway, Fiocchi, “Resistance of Crohn's disease T cells to multiple apoptotic signals is associated with a Bcl-2/Bax mucosal imbalance,” J Immunol, 163:1081-90, 1999.

Jaattela M, Mouritzen H, Elling F, Bastholm L. 1996. A20 zinc finger protein inhibits TNF and IL-1 signaling. J Immunol 156:1166-73.

Jobin and Sartor, “The IκB/NFκB system: a key determinant of mucosal inflammation and protection,” Am J Physiol Cell Physiol, 278:C451-62, 2000.

Karin and Ben-Neriah, “Phosphorylation meets ubiquitination: the control of NF-κB activity,” Annu Rev Immunol, 18:621-63, 2000.

Karin, “The beginning of the end: IκB kinase (IKK) and NF-κB activation,” J Biol Chem, 274:27339-42, 1999.

Kollias, Douni, Kassiotis, Kontoyiannis, “On the role of tumor necrosis factor and receptors in models of multiorgan failure, rheumatoid arthritis, multiple sclerosis and inflammatory bowel disease,” Immunol Rev, 169:175-194, 1999.

Koncz, Mayerhofer, Koncz-Kalman, Nawrath, Reiss, Redei, Schell, “Isolation of a gene encoding a novel chloroplast protein by T-DNA tagging in Arabidopsis thaliana,” EMBO J, (5):1337-46, 1990.

Kontoyiannis, Pasparakis, Pizarro, Cominelli, Kollias, “Impaired on/off regulation of TNF biosynthesis in mice lacking TNF AU-rich elements: implications for joint and gut-associated immunopathologies,” Immunity, 10:387-98, 1999.

Kornberg and Baker, DNA Replication, 2nd Ed., Freeman, San Francisco, 1992.

Kuhn, Lohler, Rennick Rajewsky, Muller, “Interleukin-10-deficient mice develop chronic enterocolitis,” Cell, 75:263-74, 1993.

Lambert and Borek, “X-ray-induced changes in gene expression in normal and oncogene-transformed rat cell lines,” J Natl Cancer Inst, 80(18):1492-7, 1988

Lee E G, Boone D L, Libby S, Chai S, Chien M, Lodolce J P, and Ma A. 2000. Failure to regulate TNF induced NF-κB and cell death responses in A20 deficient mice. Science 289;2350-4.

Lee, Boone, Libby, Chai, Chien, Lodolce, Ma, “Failure to regulate TNF induced NF-κB and cell death responses in A20 deficient mice,” Science, 289;2350, 2000.

Lenardo, “Interleukin-2 programs mouse alpha beta T lymphocytes for apoptosis,” Nature, 353:858-61, 1991.

Leung, Morefield, Willerford, “Regulation of lymphoid homeostasis by IL-2 receptor signals in vivo,” J Immunol, 164:3527-34, 2000.

Lindsten T, Ross A J, King A, Zong W, Rathmell J C, Shiels H A, Ulrich E, Waymire K G, Mahar P, Frauwirth K, Chen Y, Wei M, Eng V M, Adelman D M, Simon M C, Ma A, Golden J A, Evan G, Korsmeyer S J, MacGregor G R, Thompson C B. 2000. The combined functions of the pro-apoptotic Bcl-2 family members, Bak and Bax, are essential for the normal development of multiple tissues. Molecular Cell (in press).

Lindsten, Ross, King, Zong, Rathmell, Shiels, Ulrich, Waymire, Mahar, Frauwirth, Chen, Wei, Eng, Adelman, Simon, Ma, Golden, Evan, Korsmeyer, MacGregor, Thompson, “The combined functions of the pro-apoptotic Bcl-2 family members, Bak and Bax, are essential for the normal development of multiple tissues,” Molecular Cell, 6(6):1389-99, 2000.

Liu, Geboes, Colpaert, D'Haens, Rutgeerts, Ceuppens, “IL-15 is highly expressed in inflammatory bowel disease and regulates local T cell-dependent cytokine production,” J Immunol, 164:3608-15, 2000.

Liu, Hsu, Goeddel, Karin, Cell, 87:565, 1996.

Locksley R M, Killeen N, Lenardo M J. 2001. The TNF and TNF receptor superfamilies: integrating mammalian biology. Cell 104:487-501

Lodolce, Boone, Dassopoulos, Chai, Swain, Trettin, Ma, “Interleukin-15 receptor maintains lymphoid homeostasis by supporting lymphocyte homing and proliferation,” Immunity, 9;669, 1998.

Ma A, Datta M, Margosian E, Chen J, Horak I. 1995. T cells, but not B cells, are required for bowel inflammation in IL-2 deficient mice. J Ex Med 182:1567-72.

Ma A. 1997. Fas and the immunologically underprivileged intestine. Gastroenterology 113;345.

Ma, “Fas and the immunologically underprivileged intestine,” Gastroenterology, 113:345, 1997.

Ma, A, Lodolce, J P, Boone, D L. 2000. “The Pleiotropic Functions of IL-15: Not So IL-2-Like After All,” J Ex Med 191;753.

Ma, A. 2000. “IL-15 receptor: Pleiotropic Regulator of Immune Function,” Modern Aspects of Immunobiology 1;102.

Ma, Datta, Margosian, Chen, Horak, “T cells, but not B cells, are required for bowel inflammation in IL-2 deficient mice,” J Exp Med, 182:1567-72, 1995.

McCann, Choi, Yamasaki, Ames, “Detection of carcinogens as mutagens in the Salmonella/microsome test: assay of 300 chemicals,” Proc Natl Acad Sci USA, 72(12):5135-9, 1975.

Meijssen, Brandwein, Reinecker, Bhan, Podolsky, “Alteration of gene expression by intestinal epithelial cells precedes colitis in interleukin-2-deficient mice,” Am J Physiol, 274:G472-9, 1998.

Minn A J, Swain R E, Ma A, Thompson C B. 1998. Recent progress on the regulation of apoptosis by Bcl-2 family members. Ann Rev Imm 70, 245.

Neis, Gewirtz, Zeng, Young, Hobert, Karmali, Rao, Madara, “Prokaryotic regulation of epithelial responses by inhibition of IκBα ubiquitination. Science, 289:1560-3, 2000.

Neurath, Fuss, Schurmann, Pettersson, Arnold, Muller-Lobeck, Strober, Herfarth, Buschenfelde, “Cytokine gene transcription by NF-κB family members in patients with inflammatory bowel disease,” 1998. (NEED PUBLICATION)

Opipari Jr, Boguski, Dixit, “The A20 cDNA induced by tumor necrosis factor alpha encodes a novel type of zinc finger protein,” J Biol Chem, 265:14705-8, 1990.

Opipari, Boguski, and Dixit J. Biol. Chem., 265, 14705-14708, 1992.

Opipari, Jr., Hu, Yabkowitz, Dixit, J Biol Chem, 267:12424, 1992.

Oppenheimer, Schwartz, Lane, Thompson, “Functional relationship of thyroid hormone-induced lipogenesis, lipolysis, and thermogenesis in the rat,” J Clin Invest, 87(1):125-32, 1991.

Passegue E, Jochum W, Schorpp-Kistner M, Mohle-Steinlein U, Wagner E F. 2001. Chronic myeloid leukemia with increased granulocyte progenitors in mice lacking junB expression in the myeloid lineage. Cell 104:21-32.

Powrie, Correa-Oliveira, Mauze, Coffman, “Regulatory interactions between CD₄₅RB^{high} and CD45RB^{low} CD4⁺ T cells are important for the balance between protective and pathogenic cell-mediated immunity,” J Exp Med, 179:589-600, 1994.

Reinecker, MacDermott, Mirau, Dignass, Podolsky, “Intestinal epithelial cells both express and respond to interleukin 15,” Gastroenterology, 111:1706-13, 1996.

Rudolph et al, Genes Dev, 14:854, 2000.

Rutgeerts, D'Haens, Targan, Vasiliauskas, Hanauer, Present, Mayer, Van Hogezand, Braakman, DeWoody, Schaible, Van Deventer, “Efficacy and safety of retreatment with anti-tumor necrosis factor antibody (infliximab) to maintain remission in Crohn's disease,” Gastroenterology, 117:761-9, 1999.

Sadlack, Merz, Schorle, Schimpl, Feller, Horak, “Ulcerative colitis-like disease in mice with a disrupted interleukin-2 gene,” Cell, 75:253-61, 1993.

Sakai Kusugami, Nishimura, Ando, Yamaguchi, Ohsuga, Ina, Enomoto, Kimura, Yoshikai, “Interleukin 15 activity in the rectal mucosa of inflammatory bowel disease,” Gastroenterology, 114:1237-43, 1998.

Saparov, Kraus, Cong, Marwill, Xu, Elson, Weaver, “Memory/effector T cells in TCR transgenic mice develop via recognition of enteric antigens by a second, endogenous TCR,” Int Immunol, 11:1253-64, 1999.

Scheit, Nucleotide Analogs John Wiley, New York, 1980

Schmid and Adler, “NF-κB/IκB: Implications in gastrointestinal diseases,” Gastroenterology, 118;1208-28, 2000.

Sica and Chen “Modulation of the immune response through 4-1_{β}-,” Adv Exp Med Biol, 465:355-62, 2000.

Song, Rothe, Goeddel, “The tumor necrosis factor-inducible zinc finger protein A20 interacts with TRAF1/TRAF2 and inhibits NF-κB activation,” Proc Natl Acad Sci USA, 93:6721-5, 1996.

Sun, Ganchi, Ballard, Greene, “NF-κB controls expression of inhibitor IκBα: evidence for an inducible autoregulatory pathway,” Science, 259:1912-5, 1993.

Tang G, Minemoto Y, Dibling B, Purcell N H, Li Z, Karin M, Lin A. 2001. Inhibition of JNK activation through NF-kappaB target genes. Nature 414:313-7.

Targan, Hanauer, van Deventer, Mayer, Present, Braakman, DeWoody, Schaible, Rutgeerts, “A short-term study of chimeric monoclonal antibody cA2 to tumor necrosis factor alpha for Crohn's disease. Crohn's Disease cA2 Study Group,” N Engl J Med, 337:1029-35, 1997.

Tewari, Wolf, Seldin, O'Shea, Dixit, Turka, “Lymphoid expression and regulation of A20, an inhibitor of programmed cell death,” J Immunol, 154:1699-706, 1995.

Tough, Sun, Zhang, Sprent, “Stimulation of naive and memory T cells by cytokines,” Immunol Rev, 170:39-47, 1999.

Ulrich E, Kauffmann-Zeh A, Hueber A O, Williamson J, Chittenden T, Ma A, Evan G. 1997. Gene structure, cDNA sequence, and expression of murine Bak, a proapoptotic Bcl-2 family member. Genomics 44:195-200.

Ulrich, Kauffmann-Zeh, Hueber, Williamson, Chittenden, Ma, Evan, “Gene structure, cDNA sequence, and expression of murine Bak, a proapoptotic Bcl-2 family member,” Genomics, 44:195-200, 1997.

Van Antwerp, Martin, Kafri, Green, Verma, Science, 274:787, 1996.

van Kooten and Banchereau, “CD40-CD40 ligand,” J Leukoc Biol, 67:2-17, 2000.

Vincenz, and Dixi “14-3-3 Proteins Associate with A20 in an Isoform-specific Manner and Function Both as Chaperone and Adapter Molecules” J Biol. Chem. 271:33 20029-20034, 1996.

Waldmann and Tagaya, “The multifaceted regulation of interleukin-15 expression and the role of this cytokine in NK cell differentiation and host response to intracellular pathogens,” Annu Rev Immunol, 17:19-49, 1999.

Wang, Mayo, Baldwin Jr., “TNF- and cancer therapy-induced apoptosis: potentiation by inhibition of NF-κB,” Science, 274:784-7, 1996.

Wang, Mayo, Korneluk, Goeddel, Baldwin Jr., “NF-κB antiapoptosis: induction of TRAF1 and TRAF2 and c-IAP1 and c-IAP2 to suppress caspase-8 activation, Science, 281:1680-3, 1998.

Warren, McKown, Marin, Teutonico, “Isolation of mutations affecting the development of freezing tolerance in Arabidopsis thaliana (L.) Heynh,” Plant Physiol, 111(4):1011-9, 1996

Willerford, Chen, Ferry, Davidson, Ma, Alt, “Interleukin-2 receptor alpha chain regulates the size and content of the peripheral lymphoid compartment,” Immunity. 3:521-30, 1995.

Wong and Goeddel, J. Immunol, 152:1751, 1994.

Yelton, Rosok, Cruz, Cosand, Bajorath, Hellstrom, Hellstrom, Huse, Glaser, “Affinity maturation of the BR96 anti-carcinoma antibody by codon-based mutagenesis, J Immunol, 155(4):1994-2004, 1995.

Zell T, Khoruts A, Ingulli E, Bonnevier J L, Mueller D L, Jenkins M K. 2001. Single-cell analysis of signal transduction in CD4 T cells stimulated by antigen in vivo. PNAS USA 98;10805-10.

Zhang, Kovalenko, Cantarella, Wallach, Immunity, 12:301, 2000.

Zhang, Sun, Hwang, Tough, Sprent, “Potent and selective stimulation of memory-phenotype CD8⁺ T cells in vivo by IL-15,” Immunity, 8:591-9, 1998.

Zheng, Fisher, Miller, Peschon, Lynch, Lenardo, “Induction of apoptosis in mature T cells by tumour necrosis factor,” Nature, 377:348-51, 1995.

What is claimed is:

1. A method of treating a subject for a disease characterized by aberrant levels of programmed cell death and/or inflammation, comprising mediating A20 function in the subject.

2. The method of claim 1, wherein the disease is selected from the group consisting of Crohn's disease, inflammatory bowel disease, arthritis, diabetes, pulmonary inflammation, nephritis, a vascular disease mediated by endothelial cell dysfunction, a disease associated with ischemic injury, a toxin-induced liver disease or cancer.

3. The method of claim 2, wherein the disease is Crohn's disease or inflammatory bowel disease.

4. The method of claim 2, wherein the disease is a disease associated with ischemic injury.

5. The method of claim 4, wherein the ischemic injury is necrosis.

6. The method of claim 4, wherein the disease is associated with tissue ischemia.

7. The method of claim 1, wherein the disease is heart failure.

8. The method of claim 4, wherein the disease is septic shock.

9. The method of claim 2, wherein the disease is a toxin-induced liver disease.

10. The method of claim 9, wherein the disease is cirrhosis of the liver.

11. The method of claim 2, wherein the disease is cancer.

12. The method of claim 2, wherein the disease is a vascular disease mediated by endothelial cell dysfunction

13. The method of claim 11, wherein the cancer is non-Hodgkins lymphoma.

14. The method of claim 2, wherein the disease is arthritis.

15. The method of claim 2, wherein the disease is diabetes.

16. The method of claim 2, wherein the disease is pulmonary inflammation.

17. The method of claim 2, wherein the disease is nephritis

18. The method of claim 1, wherein the subject is a mammal.

19. The method of claim 18, wherein the mammal is a rodent.

20. The method of claim 19, wherein the rodent is a mouse.

21. The method of claim 18, wherein the mammal is a human.

22. The method of claim 1, wherein the subject has or is at risk of having said disease.

23. The method of claim 1, wherein the disease involves an immune response.

24. The method of claim 1, wherein the disease involves an increase or decrease in programmed cell death.

25. The method of claim 1, wherein mediating A20 function comprises providing an A20 polypeptide or a modulator of A20 activity to the subject.

26. The method of claim 25, wherein the providing comprises formulating the A20 polypeptide or modulator of A20 in a pharmaceutical composition.

27. The method of claim 26, wherein the pharmaceutical composition is administered to the subject.

28. The method of claim 27, wherein the administration comprises injection.

29. The method of claim 25, wherein providing an A20 polypeptide comprises obtaining an A20 polypeptide and incorporating it into a pharmaceutical carrier.

30. The method of claim 25, wherein providing an A20 polypeptide comprises providing a nucleic acid segment encoding the A20 polypeptide to the subject and obtaining expression of the polypeptide in the subject.

31. The method of claim 25, wherein the A20 polypeptide is a modified A20 polypeptide.

32. The method of claim 25, wherein providing the modulator of A20 function is further defined as comprising screening for a modulator of A20 function.

33. The method of claim 32, further comprising determining a modulator of A20 function and providing that modulator to the subject.

34. The method of claim 25, wherein the modulator is an agonist of A20.

35. The method of claim 25, wherein the modulator is an antagonist of A20.

36. The method of claim 25, wherein the modulator modulates a protease activity of A20.

37. The method of claim 25, wherein the modulator is a nucleic acid segment.

38. The method of claim 37, wherein the nucleic acid segment encodes an A20 polypeptide.

39. The method of claim 37, wherein the nucleic acid segment is an RNA.

40. The method of claim 39, wherein the RNA is an antisense RNA to a nucleic acid encoding A20.

41. The method of claim 25, wherein the modulator is a small molecule.

42. The method of claim 41, wherein the molecule is a protease inhibitor or agonist of A20.

43. The method of claim 1, wherein modulating A20 function comprises modulating A20 concentration in the subject.

44. The method of claim 43, wherein modulating A20 concentration is further defined as increasing A20 concentration.

45. The method of claim 43, wherein modulating A20 concentration comprises modulating A20 expression in the subject.

46. The method of claim 45, wherein modulating A20 expression comprises modulating A20 transcription.

47. The method of claim 45, wherein modulating A20 expression comprises modulating A20 translation.

48. The method of claim 43, wherein modulating A20 concentration comprises modulating the half-life of A20 in the subject.

49. The method of claim 48, wherein modulating the half-life of A20 in the subject is further defined as increasing the half-life of A20 in the subject.

50. The method of claim 1, further defined as a method of modulating a TNF mediated pathway.

51. The method of claim 50, wherein the TNF mediated pathway is an NF-κB pathway, a JNK pathway, or a programmed cell death pathway.

52. The method of claim 51, wherein the TNF mediated pathway is the JNK pathway.

53. The method of claim 1, further defined as a method of modulating an immune response.

54. The method of claim 53, further defined as a method of modulating an innate immune response.

55. The method of claim 53, further defined as inhibiting an immune response.

56. The method of claim 51, further defined as a method of inhibiting TNF induced programmed cell death.

57. The method of claim 1, further defined as a method of inhibiting both NF-κB activity and programmed cell death.

58. The method of claim 53, further defined as increasing A20 activity.

59. The method of claim 53, further defined as inducing an immune response.

60. The method of claim 59, further defined as a method of preventing or treating a disease state involving a lack of an immune response.

61. A method of treating a subject for Crohn's disease and/or inflammatory bowel disease, comprising increasing A20 function in the subject.

62. The method of claim 61, wherein the subject is a mammal.

63. The method of claim 62, wherein the mammal is a rodent.

64. The method of claim 63, wherein the rodent is a mouse.

65. The method of claim 62, wherein the mammal is a human.

66. The method of claim 61, wherein the subject has or is at risk of having said disease.

67. The method of claim 66, wherein the disease involves an increase in programmed cell death.

68. The method of claim 61, wherein increasing A20 function comprises providing an A20 polypeptide or a modulator of A20 activity to the subject.

69. The method of claim 68, wherein the provision comprises formulating the A20 polypeptide or modulator of A20 in a pharmaceutical composition.

70. The method of claim 68, wherein providing an A20 polypeptide comprises providing a nucleic acid segment encoding the A20 polypeptide to the subject and obtaining expression of the polypeptide in the subject.

71. The method of claim 68, wherein the A20 polypeptide is a modified A20 polypeptide.

72. The method of claim 68, wherein providing the modulator of A20 function is further defined as comprising screening for a modulator of A20 function.

73. The method of claim 68, wherein the modulator is an agonist of A20.

74. The method of claim 68, wherein the modulator is a small molecule.

75. The method of claim 61, further defined as a method of inhibiting programmed cell death in an intestinal epithelial cell.

76. A method of screening for modulators of an A20-mediated process comprising obtaining a candidate substance and contacting a subject that is homozygous for an A20 negative allele with the candidate substance.

77. The method of claim 76, wherein the subject is a mammal.

78. The method of claim 77, wherein the mammal is a mouse.

79. The method of claim 76, wherein the candidate substance is a small molecule.

80. The method of claim 76, wherein the candidate substance is a nucleic acid segment.

81. The method of claim 76, wherein the candidate substance is a polypeptide.

82. The method of claim 76, wherein screening comprises observing an A20-mediated process following said contacting.
